SEQUENCE LISTING (6 sequences)

SEQ ID NO: 1 - protein, 8 aa, synthetic construct
  source 1..8: note = Peptide tag
DYKDDDDK

SEQ ID NO: 2 - DNA, 21 bp, synthetic construct
  source 1..21: mol_type = other DNA; note = Nucleotide oligo
aaaaaaaaaaaaaaaaaaaaa

SEQ ID NO: 3 - DNA, 32 bp, synthetic construct
  source 1..32: mol_type = other DNA; note = Nucleotide oligo
acgataagggtagcggctccaaaaaaaaaaaa

SEQ ID NO: 4 - DNA, 33 bp, synthetic construct
  source 1..33: mol_type = other DNA; note = Primer
ttttttttttttttttcatgagacccactaacg

SEQ ID NO: 5 - DNA, 18 bp, synthetic construct
  source 1..18: mol_type = other DNA; note = Primer
acgacgctcttccgatct

SEQ ID NO: 6 - DNA, 18 bp, synthetic construct
  source 1..18: mol_type = other DNA; note = Primer
ggagccgctacccttatc

(19) United States
(12) Patent Application Publication
(10) Pub. No.: US 2024/0327822 A1
(43) Pub. Date: Oct. 3, 2024

(54) MATERIALS AND METHODS TO COMPREHENSIVELY DEFINE ADAPTIVE IMMUNE RESPONSES

(71) Applicant: The University of Hong Kong, Hong Kong (CN)

(72) Inventors: Ren Sun, Hong Kong (CN); Xiangzhi Meng, Hong Kong (CN)

(21) Appl. No.: 18/579,137

(22) PCT Filed: Aug. 9, 2022
(86) PCT No.: PCT/CN2022/111160

§ 371 Date: Jan. 12, 2024

Related U.S. Application Data
(60) Provisional application No. 63/232,507, filed on Aug. 12, 2021.

(51) Int. Cl. C12N 15/10; A61K 39/215; C07K 14/74

(52) U.S. Cl. CPC C12N 15/1062; A61K 39/215; C07K 14/70539

(57) ABSTRACT

Methods for detecting adaptive immune responses to pathogens or self-antigens by antibody or B cell or T cell binding to antigenic epitopes have been established. The methods inform functional and structural interactions between immune receptors and antigens, identify potential therapeutic targets and guide vaccine development. The methods employ high throughput modified mRNA-display or variations of droplet display to determine single epitope-specific antibody and B- and T- cell receptor sequences at the genomic scale at single epitope and single amino acid resolution. In some forms, the methods collect and integrate the data to provide a database of an adaptive immunity profile for a human or animal subject. In some forms, the methods identify and record changes in an immunity profile over different time points to reflect immunological responses in a subject. The methods provide high resolution immunity profiles of immune responses at the genomic level for diagnostic, prophylactic, and therapeutic applications.

This international patent application claims the benefit of U.S. Provisional Patent Application No. 63/232,507 filed on Aug. 12, 2021, the entire content of which is incorporated by reference for all purpose.

### FIELD OF THE INVENTION

The invention is generally directed to detecting and monitoring immune responses, and in particular, characterizing adaptive immunity in response to antigen recognition.

### BACKGROUND OF THE INVENTION

Recent advances in next-generation sequencing (NGS) enable high-throughput identification of the sequences of B and T cell repertoires at the single cell level (Busse, C. E. et al., Eur J Immunol 44, 597-603, (2014); Tan, Y. C. et al., Clin Immunol 151, 55-65, (2014); DeKosky, B. J. et al., Nat Biotechnol 31, 166-169, (2013)). However, the annotation of B and T cell receptor sequences together with their cognate epitopes generally requires generation of individual B and T cell clones (DeFalco, J. et al., Clin Immunol 187, 37-45, (2018); Setliff, I. et al., Cell Host Microbe 23, 845-854 e846, (2018)), which is costly and highly time-consuming. Even though a handful of studies have been performed recently to profile the receptor sequences with their corresponding epitopes using single-cell sequencing technology, these methods are practical only on a low-throughput scale. For example, LIBRA-seq (linking B cell receptor to antigen specificity through sequencing; Setliff, I. et al., Cell 179, 1636-1646 e1615, (2019)) and TetTCR-seq (tetramer-associated T-cell receptor sequencing; Zhang, S. Q. et al., Nat Biotechnol, (2018)) represent the most advanced technologies. However, the LIBRA-seq method relies on purification of individual proteins as epitopes; while TetTCR-seq depends on preparation of MHC complex individually loaded with each specific peptide, thus limiting the throughput.

Monoclonal antibodies are used as important prophylactic and therapeutic agents in the clinic and attempts to overcome the low-throughput limitations of using traditional single B cell clone generation for novel antibody development have developed screening technologies to identify B cell receptor (antibody) sequences targeting specific antigens. For example, natively paired human B cell receptor (BCR) heavy- and light-chain amplicons can be expressed and screened in the form of antigen-binding fragments (Fab), or single-chain variable fragments (scFv) in a yeast display or phage display system (Adler, A. S. et al., MAbs 10, 431-443, (2018); Adler, A. S. et al., MAbs 9, 1282-1296, (2017); Wang, B. et al., MAbs 8, 1035-1044, (2016)). Although these various antibody discovery technologies have led to the identification of potential neutralizing antibodies, their efficiency remains limited by the number of epitopes that can be simultaneously screened.

In an individual, the potential repertoire of B cells is of the order of 10⁷ (minimal estimate) up to 1015 (maximal theoretical number of possible combinations). The repertoire for T Cell Receptors (TCR) is estimated to be in the similar range.

The diversity and target specificity of the repertoire are critical determinants of the immune responses, underlying the healthy condition of an individual, impacting the outcome of infection, cancer, and autoimmunity. The diversity and specificity of immune repertoires is critical for the host to defend against diverse pathogens, mutations in cancer cells and alteration of self-antigens, constrained with the requirement of differentiating from the “self”. To assist understanding of fundamental immunological processes, the diversity and specificity of B cells and T cells should be determined in development and disease, together with their corresponding epitopes. Currently available methodologies lack sufficient high-throughput efficacy and resolution to characterize B cell and T cell epitopes on a whole-organism level.

There is a need for developing efficient platforms for coupling B cell receptor or T cell receptor sequences with their epitopes in a high-throughput and high-resolution manner. There is also a need for developing efficient platforms for accurate estimation/measurement of BCR and TCR repertoires for an individual at specific time points throughout an infection. Therefore, it is an object of the invention to simultaneously characterize multiple B cells and T cells, together with their corresponding multiple epitope specificities. It is a further object of the invention to provide high-throughput methods to screen and select individual B cells or T cells, based upon antigen recognition and/or epitope specificity. It is a further object of the invention to provide databases of antibody, B cell and T cell sequences in real time throughout the course of an immune response in an individual.

### SUMMARY OF THE INVENTION

Methods for generating immunological profiles have been established. The methods exploit the discovery that the dynamics of adaptive immune response against infection or self-antigens can be reflected by antibody binding activity and B/T cell receptor sequences binding to pathogenic epitopes.

Methods for characterizing an immune response to an antigen in an individual, including generating an immunological profile for an immune response in the individual to the antigen are provided. Typically, the immunological profile includes a multiplicity of nucleic acid sequences of immune receptors from the individual, or a multiplicity of nucleic acid sequences from the antigen, or a combination thereof, and generating the immunological profile includes selecting at least one target binding nucleic acid-protein fusion from a library of two or more nucleic acid-protein fusions. The target is one or more T cell receptors (TCR) of a target T cell, a B cell receptor (BCR) of a target B cell, or antigen binding domain of a target immunoglobulin (FAb). Typically, the immunological profile includes the nucleic acid sequence of the selected nucleic acid-protein fusion, or the nucleic acid sequence of the target, or both. In some forms, the nucleic acid-protein fusion is generated by mRNA Display of antigen RNA/DNA, or a multiplicity of antigen RNA/DNA fragments. In an exemplary form, RNA Display comprises: the steps of (i) performing in vitro transcription on the antigen DNA or multiplicity of DNA fragments to obtain antigen mRNA(s); (ii) covalently linking the antigen mRNA(s) at the 3′ end to a protein acceptor selected from puromycin, tRNA-puromycin conjugate, phenylalanyl-adenosine, tyrosyl adenosine, alanyl adenosine, phenylalanyl 3′ deoxy 3′ amino adenosine, alanyl 3′ deoxy 3′ amino adenosine, and tyrosyl 3′ deoxy 3′ amino adenosine to obtain a ligated antigen mRNA or a ligated antigen mRNA/DNA hybrid; and (iii) performing in vitro translation and reverse transcription on the ligated antigen mRNA(s) to obtain a nucleic acid-protein fusion(s). Typically, the protein of the nucleic acid-protein fusion is encoded by the nucleic acid of the nucleic acid-protein fusion. In some forms, the DNA encoding the antigen is provided by a method selected from on-chip DNA synthesis technologies, synthesis of regular oligonucleotides containing mutant cassettes, and fragmentation of genomic or cDNAs, or combinations thereof.

In some forms, the antigen DNA comprises: nucleic acids from one or more of a protozoan antigen, a viral antigen, a bacterial antigen, a fungal antigen, a nematode antigen, a human auto-antigen, a human tumor antigen, and an environmental allergen; naturally occurring sequences, mutations of the natural sequences, or de novo designed sequences. In some forms, the DNA fragments include a promoter sequence, Kozak sequence and a sequence encoding a first peptide tag at the 5′ end, and a sequence encoding a second peptide tag at the 3′ end. In preferred forms, the nucleic acid-protein fusion(s) are purified by affinity or identified using the first and/or second peptide tags. Typically, the nucleic acid-protein fusion library includes at least 10³ nucleic acid-protein fusions, at least 10⁶ nucleic acid-protein fusions, or at least 10¹⁰ nucleic acid-protein fusions.

In some forms, the immunological profile includes peptide epitopes of the antigen that are bound by immunoglobulin from the individual. Typically, the epitopes for T cell receptors are between 5 and 1000 amino acids in length, more frequently between 7 and 100 amino acids in length, more preferably between 7 and 12 amino acids in length. In some forms, the epitopes are identified by methods including (i) immobilizing the immunoglobulin to obtain an immobile phase; (ii) contacting the immobile phase with the nucleic acid-protein fusions under conditions that allow for binding of the nucleic acid-protein fusions to the immunoglobulin within the immobile phase to obtain target binding nucleic acid-protein fusions; and (iii) characterizing the target binding nucleic acid-protein fusions.

In some forms, the target-binding nucleic acid-protein fusions are isolated by binding to immobilized immunoglobulins selected from IgG1, IgG2, IgG3, IgG4, IgM, IgE, IgA, and other Ig subtypes, or combinations thereof, and the immunological profile includes information identifying the class of immunoglobulin that bound to each target-binding nucleic acid-protein fusion. In some forms, target-binding nucleic acid protein fusions and/or target B cells and/or target T cells include one or more synthetic nucleic acid sequences comprising: bar-code information relating to the sample. In some forms, synthetic nucleic acid sequences comprising: bar-code information for target B cell(s) or target T cell(s) are associated with a bead or other matrix with which the target B cell(s) or target T cell(s) is also associated. In particular forms, the antigen comprises: the SARS-CoV-2 virus. For example, in some forms, the immunological profile includes the nucleic acid sequences of one or more target-binding epitope(s) of the SARS-COV-2 virus, or the nucleic acid sequences of one or more BCR that selectively binds an epitope of the SARS-COV-2 virus, or the nucleic acid sequences of one or more TCR that selectively binds an epitope of the SARS-COV-2 virus, or combinations thereof.

Typically, characterizing the target binding nucleic acid-protein fusions includes isolating target binding nucleic acid-protein fusions and sequencing the nucleic acid of the nucleic acid-protein fusions. In some forms, isolating the target binding nucleic acid-protein fusions includes eluting the target binding nucleic acid-protein fusion from the immunoglobulin, eluting the target binding nucleic acid-protein fusion from the immunoglobulin with competitive binder or with an enzyme (IdeZ as an example), and sequencing comprises: PCR amplification and/or bar-coding of the nucleic acid of the target binding nucleic acid-protein fusions and preparation of a sequencing library. In some forms, the preparation of a sequencing library includes one or more of pooling the nucleic acids, end-repair, dA-tailing, adaptor ligation and PCR amplification. In some forms, the methods include one or more steps for correlating the peptide epitopes of the antigen within the immunological profile with one or more disease states or indications.

In some forms, the methods include one or more steps to characterize the target B cell(s). In some forms, the B cells are isolated from a source selected from fresh or properly frozen blood, purified lymphocytes, tissues using selection kits, and organs staining by antibodies recognizing B cell marker. In an exemplary form, characterizing the target B cell(s) includes (i) labeling the nucleic acid-protein fusions with a detectable label; (ii) contacting the labeled nucleic acid-protein fusions with a multiplicity of B cell(s) from the individual, where the contacting is under conditions that allow for binding of the labeled nucleic acid-protein fusions to the target B cell(s); and (iii) detecting target B cell(s) bound to the labeled nucleic acid-protein fusions; (iv) isolating the target B cell(s); and (v) obtaining the nucleic acid sequence of the BCR, and optionally one or more other genes of the target B cell(s). Typically, labeling the nucleic acid-protein fusions with a detectable label includes performing reverse transcription of the peptide/protein-mRNA fusion complex using biotin-modified primers, where the primers anneal to both the oligo dA and constant regions of the mRNA, and where the label includes fluorophore-conjugated streptavidin that is bound to biotin on the cDNA. Typically, the target B cells are isolated by flow-cytometry, with magnetic beads, with other affinity beads or with other affinity surfaces.

In some forms, characterizing the target B cell(s) further includes preparing one or more databases of a multiplicity of nucleic acid sequences from one or more target B cell, optionally where the multiplicity of nucleic acid sequences comprises: the target B cell transcriptome, including but not limited to BCR. In some forms, the molecular profile includes (i) the nucleic acid sequences of a multiplicity of target binding nucleic acid-protein fusions; and (ii) target B cell data. Typically, target B cell data includes nucleic acid sequences from a multiplicity of target B cell(s) that are bound by the target binding nucleic acid-protein fusions. In particular forms, the methods associate target B cell data within the immunological profile with an immune response to one or more vaccines, infections, or other physiological/pathological conditions associated with the antigen.

In some forms, the methods include one or more steps to characterize the target T cell(s). In some forms, the T cells are isolated from a source selected from fresh or properly frozen blood, purified lymphocytes, tissues using selection kits, and organs staining by antibodies recognizing T cell marker(s). In some forms, characterizing the target T cell(s) includes (i) loading the nucleic acid-protein fusions into major histocompatibility complex (MHC) molecules, where the MHC is labeled with a detectable label, to form a multiplicity of labeled MHC/nucleic acid-protein fusions; (ii) contacting the labeled MHC/nucleic acid-protein fusions with a multiplicity of T cell(s) from the individual, where the contacting is under conditions that allow for binding of the labeled MHC/nucleic acid-protein fusions to the target T cell(s); (iii) detecting the target T cell(s) bound to the labeled nucleic acid-protein fusions; (iv) isolating the target T cell(s); and (iv) obtaining the nucleic acid sequence of the TCR, and optionally one or more other genes of the target T cell(s). Typically, characterizing the target T cell(s) includes preparing one or more databases of a multiplicity of nucleic acid sequences from one or more target T cell, optionally where the multiplicity of nucleic acid sequences includes the target T cell transcriptome, including but not limited to TCR. In preferred forms, the molecular profile includes (i) the nucleic acid sequences of a multiplicity of target binding nucleic acid-protein fusions; and (ii) target T cell data, where the target T cell data includes the nucleic acid sequences of a multiplicity of target T cell(s) that are bound by the target binding nucleic acid-protein fusions.

In some forms, the methods associate target T cell data within the immunological profile with an immune response to one or more vaccines or infections associated with the antigen. In some forms, the immunological profile comprises: at least 5% of the BCR repertoire of the individual, or at least 5% of the TCR repertoire of the individual, or at least 5% of the BCR and TCR repertoire of the individual specific for the antigen. In some forms, the immunological profile comprises: at least 50% of the BCR repertoire of the individual, or at least 50% of the TCR repertoire of the individual, or at least 50% of the BCR and TCR repertoire of the individual specific for the antigen. In some forms, the immunological profile comprises: at least 100 different BCR clones of the individual, or at least 100 different TCR clones of the individual, or at least 100 different BCR and TCR clones of the individual specific for the antigen. In some forms, the immunological profile comprises: at least 1000 different BCR clones of the individual, or at least 1000 different TCR clones of the individual, or at least 1000 different BCR and TCR clones of the individual specific for the antigen. In preferred forms, the immunological profile includes (i) the nucleic acid sequence of target-binding nucleic acid-protein fusions; (ii) target B cell data; and (iii) target T cell data. In some forms, the methods include performing one or more computations on the immunological profile to identify one or more criteria within a multiplicity of nucleic acid sequences of immune receptors from the individual, or a multiplicity of nucleic acid sequences from the antigen. In an exemplary form, one criterion is identifying an autoantigen within the multiplicity of nucleic acid sequences from the antigen and the corresponding TCRs and BCRs. The methods optionally include identifying or assisting selection of anti-autoimmune therapy based on the identification of an autoantigen. In another exemplary form, one criterion is identifying a tumor antigen within the multiplicity of nucleic acid sequences from the antigen and the corresponding TCRs and BCRs. The methods optionally include identifying or assisting selection of anti-cancer therapy based on the identification of a tumor antigen. In another exemplary form, one criterion is identifying a transplantation-associated immune response in the individual. The methods optionally include identifying or assisting selection of therapy based on the identification of autoantigens associated with transplant rejection. In another exemplary form, one criterion is identifying or diagnosing a disease in the individual, where the identifying is based on the identification of immunoglobulins, BCRs and/or TCRs associated with the immune response.

Methods of making and using enhanced vaccines against an antigen are provided. Typically, the methods employ one or more steps to characterize B cells specific for the antigen, or T cells specific for the antigen, or antibodies specific for the antigen, or combinations thereof within a subject. The methods provide enhanced vaccines with improved specificity, and antigen cross-reactivity, whilst preventing the development or reducing the severity of autoimmunity in the subject. In some forms, the methods identify epitope-specific sequences amongst immune receptors in the subject for a multiplicity of epitopes within the antigen; determine which one or more of the multiplicity of epitopes for the antigen have the highest number of epitope-specific sequences in the subject; and preparing the vaccine including one or more of the epitopes having the highest number of epitope-specific sequences in the subject. In a particular form, the methods identify epitope-specific T cell receptor sequences in the subject for a multiplicity of epitopes within the antigen; determine which one or more of the multiplicity of epitopes for the antigen have the highest number of epitope-specific T cell receptor sequences in the subject; and prepare the vaccine including one or more of the epitopes having the highest number of epitope-specific T cell receptor sequences in the subject. In another form, the methods identify a multiplicity of antibody epitopes within the antigen by mRNA display; and prepare the vaccine including one or more of the antibody epitopes. In other forms, the methods include identifying epitope-specific B cell receptor sequences in the subject for a multiplicity of epitopes within the antigen; determining which one or more of the multiplicity of epitopes for the antigen have the highest number of epitope-specific B cell receptor sequences in the subject; and preparing the vaccine for the subject including one or more of the epitopes determined as having the highest number of epitope-specific B cell receptor sequences in the subject.

Preferred embodiments of the invention are as follows.

1. A method for characterizing an immune response to an antigen or a set of antigens in an individual, comprising: generating an immunological profile for an immune response in the individual to the antigen or a set of antigens,
wherein the immunological profile comprises: a multiplicity of nucleic acid sequences of immune receptors from the individual, or a multiplicity of nucleic acid sequences from the antigen, or a combination thereof, and
wherein generating the immunological profile comprises: selecting at least one target binding nucleic acid-protein fusion from a library of two or more nucleic acid-protein fusions,
wherein the target is one or more T cell receptors (TCR) of a target T cell, a B cell receptor (BCR) of a target B cell, or antigen binding domain of a target immunoglobulin (FAb), and
wherein the immunological profile comprises: the nucleic acid sequence of the selected nucleic acid-protein fusion, or the nucleic acid sequence of the target, or both.

2. The method of embodiment 1, wherein the nucleic acid-protein fusion is generated by RNA display of antigen DNA, or a multiplicity of antigen DNA fragments, wherein the RNA display comprises: the steps of
(i) performing in vitro transcription on the antigen DNA or multiplicity of fragments to obtain antigen mRNA(s);
(ii) covalently linking the antigen mRNA(s) at the 3′ end to a protein acceptor selected from the group consisting of puromycin, tRNA-puromycin conjugate, phenylalanyl-adenosine, tyrosyl adenosine, alanyl adenosine, phenylalanyl 3′ deoxy 3′ amino adenosine, alanyl 3′ deoxy 3′ amino adenosine, and tyrosyl 3′ deoxy 3′ amino adenosine to obtain a ligated antigen mRNA; and
(iii) performing in vitro translation and reverse transcription on the ligated antigen mRNA(s) to obtain a nucleic acid-protein fusion(s),
wherein the protein of the nucleic acid-protein fusion is encoded by the nucleic acid of the nucleic acid-protein fusion.

3. The method of embodiment 2, wherein the DNA encoding the antigen is provided by a method selected from the group consisting of on-chip DNA synthesis technologies, synthesis of regular oligonucleotides containing mutant cassettes, and fragmentation of genomic or cDNAs, or combinations thereof.

4. The method of any one of embodiments 1-3, wherein the antigen DNA comprises: nucleic acids from a source selected from the group consisting of a protozoan antigen, a viral antigen, a bacterial antigen, a fungal antigen, a nematode antigen, a human auto-antigen, a human tumor antigen, and an environmental allergen.

5. The method of any one of embodiments 1-4, wherein the DNA fragments comprise a promoter sequence, Kozak sequence and a sequence encoding a first peptide tag at the 5′ end, and a sequence encoding a second peptide tag at the 3′ end.

6. The method of embodiment 5, wherein the nucleic acid-protein fusion(s) are purified by affinity using the first and/or second peptide tags.

7. The method of embodiment 1, wherein the library comprises: at least 10³ nucleic acid-protein fusions, at least 10⁶ nucleic acid-protein fusions, or at least 10¹⁰ nucleic acid-protein fusions.

8. The method of any one of embodiments 1-7, wherein the immunological profile comprises: peptide epitopes of the antigen that are bound by immunoglobulin from the individual.

9. The method of embodiment 8, wherein the epitopes are between 5 and 100 amino acids in length, preferably between 9 and 60 amino acids in length, more preferably between 30 and 50 amino acids in length.

10. The method of embodiment 8 or 9, wherein the epitopes are identified by a method comprising:
(i) immobilizing the immunoglobulin to obtain an immobile phase;
(ii) contacting the immobile phase with the nucleic acid-protein fusions under conditions that allow for binding of the nucleic acid-protein fusions to the immunoglobulin within the immobile phase to obtain target binding nucleic acid-protein fusions; and
(iii) characterizing the target binding nucleic acid-protein fusions.

11. The method of embodiment 10, wherein the target-binding nucleic acid-protein fusions are isolated by binding to immobilized immunoglobulins selected from the group consisting of IgG1, IgG2, IgG3, IgG4, IgM, IgE, and IgA, or combinations thereof, and
wherein the immunological profile comprises: information identifying the class of immunoglobulin that bound to each target-binding nucleic acid-protein fusion.

12. The method of embodiment 10 or 11, wherein characterizing the target binding nucleic acid-protein fusions comprises: isolating target binding nucleic acid-protein fusions and sequencing the nucleic acid of the nucleic acid-protein fusions.

13. The method of embodiment 12, wherein isolating the target binding nucleic acid-protein fusions comprises: eluting the target binding nucleic acid-protein fusion from the immunoglobulin, and
wherein the sequencing comprises: PCR amplification and/or bar-coding of the nucleic acid of the target binding nucleic acid-protein fusions and preparation of a sequencing library.

14. The method of embodiment 13, wherein the preparation of a sequencing library comprises: one or more of pooling the nucleic acids, end-repair, dA-tailing, adaptor ligation and PCR amplification.

15. The method of any one of embodiments 8-14, further comprising: correlating the peptide epitopes of the antigen or the set of antigens within the immunological profile with one or more disease states or indications.

16. The method of any one of embodiments 1-15, further comprising: the step of characterizing the target B cell(s).

17. The method of embodiment 16, wherein the target B cell(s) are isolated from a source selected from the group consisting of fresh or properly frozen blood, purified lymphocytes, tissues using selection kits, and organs staining by antibodies recognizing B cell marker.

18. The method of embodiment 17, wherein characterizing the target B cell(s) comprises:
(i) labeling the nucleic acid-protein fusions with a detectable label;
(ii) contacting the labeled nucleic acid-protein fusions with a multiplicity of B cell(s) from the individual,
wherein the contacting is under conditions that allow for binding of the labeled nucleic acid-protein fusions to the target B cell(s); and
(iii) detecting target B cell(s) bound to the labeled nucleic acid-protein fusions;
(iv) isolating the target B cell(s); and
(v) obtaining the nucleic acid sequence of the BCR, and optionally one or more other genes of the target B cell(s).

19. The method of embodiment 18, wherein labeling the nucleic acid-protein fusions with a detectable label comprises: performing reverse transcription of the peptide/protein-nucleic acid-fusion complex using biotin-modified primers,
wherein the primers anneal to both the oligo dA or oligo A and constant regions of the mRNA, and
wherein the label comprises: fluorophore-conjugated streptavidin that is bound to biotin on the cDNA.

20. The method of embodiment 18, wherein the target B cell(s) are isolated by flow-cytometry, magnetic beads, or other immobilized surfaces.

21. The method of any one of embodiments 16-20, wherein characterizing the target B cell(s) further comprises: preparing one or more databases of a multiplicity of nucleic acid sequences from one or more target B cells,
optionally wherein the multiplicity of nucleic acid sequences comprises: the target B cell transcriptome, including but not limited to the BCR sequences.

22. The method of any one of embodiments 1-21, wherein the molecular profile comprises:
(i) the nucleic acid sequences of a multiplicity of target binding nucleic acid-protein fusions; and
(ii) target B cell data,
wherein the target B cell data comprises: nucleic acid sequences of a multiplicity of target B cell(s) that are bound by the target binding nucleic acid-protein fusions.

23. The method of embodiment 1 or 22, further comprising: associating target B cell data within the immunological profile with an immune response to one or more vaccines or infections associated with the antigen.

24. The method of any one of embodiments 1-23, further comprising:
characterizing the target T cell(s).

25. The method of embodiment 24, wherein characterizing the target T cell(s) comprises:
(i) loading the nucleic acid-protein fusions into major histocompatibility complex (MHC) molecules, wherein the MHC is labeled with a detectable label, to form a multiplicity of labeled MHC/nucleic acid-protein fusions;
(ii) contacting the labeled MHC/nucleic acid-protein fusions with a multiplicity of T cell(s) from the individual, wherein the contacting is under conditions that allow for binding of the labeled MHC/nucleic acid-protein fusions to the target T cell(s); and
(iii) detecting the target T cell(s) bound to the labeled nucleic acid-protein fusions;
(iv) isolating the target T cell(s); and
(v) obtaining the nucleic acid sequence of the TCR, and optionally one or more other genes of the target T cell(s).

26. The method of embodiment 24 or 25, wherein characterizing the target T cell(s) further comprises: preparing one or more databases of a multiplicity of nucleic acid sequences from one or more target T cell,
optionally wherein the multiplicity of nucleic acid sequences comprises: the target T cell transcriptome.

27. The method of any one of embodiments 1-26, wherein the molecular profile comprises:
(i) the nucleic acid sequences of a multiplicity of target binding nucleic acid-protein fusions; and
(ii) target T cell data,
wherein the nucleic acid sequences of a multiplicity of target T cell(s) that are bound by the target binding nucleic acid-protein fusions.

28. The method of any one of embodiments 1, 23, and 27, further comprising: associating target T cell data within the immunological profile with an immune response to one or more vaccines or infections associated with the antigen.

29. The method of any one of embodiments 1, 23, and 27, wherein the immunological profile comprises: at least 50% of the BCR repertoire of the individual, or at least 50% of the TCR repertoire of the individual, or at least 50% of the BCR and TCR repertoire of the individual specific for the antigen.

30. The method of any one of embodiments 1, 23, and 27, wherein the immunological profile comprises:
(i) the nucleic acid sequence of target-binding nucleic acid-protein fusions;
(ii) target B cell data; and
(iii) target T cell data.

31. The method of any one of embodiments 1, 23, 27, and 30, further comprising: performing one or more computations on the immunological profile to identify one or more criteria within a multiplicity of nucleic acid sequences of immune receptors from the individual, or a multiplicity of nucleic acid sequences from the antigen.

32. The method of embodiment 31, wherein one criterion is identifying an auto-antigen or a set of antigens within the multiplicity of nucleic acid sequences from the antigen, optionally wherein the method further comprises: identifying or assisting selection of anti-autoimmune therapy based on the identification of an auto-antigen.

33. The method of embodiment 31, wherein one criterion is identifying a tumor antigen within the multiplicity of nucleic acid sequences from the antigen, and
optionally wherein the method further comprises: identifying or assisting selection of anti-cancer therapy based on the identification of a tumor antigen.

34. The method of embodiment 31, wherein one criterion is identifying a transplantation-associated immune response in the individual, and
optionally wherein the method further comprises: identifying or assisting selection of therapy based on the identification of auto-antigens associated with transplant rejection.

35. The method of embodiment 31, wherein one criterion is identifying or diagnosing a disease in the individual,
wherein the identifying is based on the identification of immunoglobulins, BCRs and/or TCRs associated with the immune response.

36. The method of any one of embodiments 1-35, wherein the target-binding nucleic acid-protein fusions and/or target B cells and/or target T cells comprise one or more synthetic nucleic acid sequences comprising: bar-code information relating to the sample.

37. The method of embodiment 36, wherein the synthetic nucleic acid sequences comprising: bar-code information for target B cell(s) or target T cell(s) are associated with a bead or other matrix with which the target B cell(s) or target T cell(s) is associated.

38. The method of any one of embodiments 1-37, wherein the antigen comprises: the SARS-COV-2 virus.

39. The method of embodiment 38, wherein the immunological profile comprises: the nucleic acid sequences of one or more target-binding epitope(s) of the SARS-COV-2 virus, or the nucleic acid sequences of one or more BCR that selectively binds an epitope of the SARS-COV-2 virus, or the nucleic acid sequences of one or more TCR that selectively binds an epitope of the SARS-COV-2 virus, or combinations thereof.

40. A method for identifying antibody epitopes by mRNA display, comprising: preparation of an mRNA-display epitope library from an antigen; and immuno-capture of the mRNA-display epitope library.

41. The method of embodiment 40, wherein preparation of the mRNA-display epitope library comprises:
(i) preparation of a double-stranded DNA library from an antigen,
wherein the double-stranded DNA library comprises: a multiplicity of fragments comprising: a promoter sequence, a nucleic acid motif that functions as a protein translation initiation site, a sequence encoding a first peptide tag at the 5′ end; and a sequence encoding a second peptide tag at the 3′ end;
(ii) preparation of a peptide/protein-mRNA fusion complex from the double-stranded DNA library; and
(iii) cDNA synthesis on the peptide/protein-mRNA fusion complex to generate a peptide/protein-mRNA-cDNA fusion complex.

42. The method of embodiment 41, wherein the promoter sequence is the T7 promoter sequence, wherein the protein translation initiation site is a Kozak sequence, the first peptide tag is a DYKDDDDK (SEQ ID NO. 1) tag, and wherein the second peptide tag is a Strep-tagII.

43. The method of embodiment 41 or 42, wherein preparation of a peptide/protein-mRNA fusion complex comprises:
(a) in vitro Transcription of the double-stranded DNA to produce RNA,
(b) ligation of the RNA with a poly-dA DNA to produce ligated RNA/DNA,
(c) purification of ligated RNA
(d) in vitro translation of the ligated RNA/DNA to produce a peptide/protein-mRNA fusion complex, and
(e) purification of the peptide/protein-mRNA fusion complex.

44. The method of any one of embodiments 41-43, wherein cDNA synthesis to generate peptide/protein-mRNA-cDNA fusion comprises: reverse transcription of the peptide/protein-mRNA fusion to produce a peptide/protein-mRNA-cDNA fusion.

45. The method of embodiment 40, wherein immuno-capture of mRNA-display epitope library comprises:
(iv) Capture of antibody from the subject onto a solid matrix;
(v) Immuno-capture of peptide/protein-mRNA-cDNA fusion;
(vi) Elution of peptide/protein-mRNA-cDNA fusion;
(vii) PCR amplification and barcoding of the peptide/protein-mRNA-cDNA fusion,
wherein the barcodes are specific to each sample; and
(viii) Preparation of a sequencing library.

46. The method of embodiment 45, wherein capture of antibody from the subject onto a solid matrix comprises: one or more steps of
(g) coating the solid matrix with antibody-binding ligands,
wherein the ligands specifically bind to antibodies;
(h) blocking the ligands to provide blocked antibody-binding ligands,
wherein the blocking prevents non-specific binding of proteins to the antibody-binding ligands;
(i) antibody capture by the blocked antibody-binding ligands to provide antibody-ligand complexes; and
(j) washing of the antibody-ligand complexes to provide pure antibody-ligand complexes.

47. The method of embodiment 45 or 46, wherein immuno-capture of peptide/protein-mRNA-cDNA fusion comprises: mixing the peptide/protein-mRNA-cDNA fusion with the blocked antibody binding ligands under conditions suitable for binding of the peptide/protein-mRNA-cDNA fusion with the blocked antibody binding ligands.

48. The method of any one of embodiments 45-47, wherein Preparation of a sequencing library comprises: one or more of end-repair, dA-tailing, adaptor ligation and PCR amplification of the peptide/protein-mRNA-cDNA fusion.

49. A method for identifying epitope-specific B cell receptor sequences for an antigen, comprising:
preparation of a labeled mRNA-display epitope library from the antigen;
preparation of B cells labeled with mRNA-display epitope library;
preparation of bar-coded beads;
encapsulation of single B cells and a single-beads into droplet; and
sequencing the library of encapsulate B cells.

50. The method of embodiment 49, wherein preparation of the labeled mRNA-display epitope library comprises:
(i) preparation of a double-stranded DNA library from an antigen,
wherein the double-stranded DNA library comprises: a multiplicity of fragments comprising: a promoter sequence, a nucleic acid motif that functions as a protein translation initiation site, a sequence encoding a first peptide tag at the 5′ end; and a sequence encoding a second peptide tag at the 3′ end;
(ii) preparation of a peptide/protein-mRNA fusion complex from the double-stranded DNA library; and
(iii) cDNA synthesis on the peptide/protein-mRNA fusion complex with fluorescent labeling to generate a labeled peptide/protein-mRNA-cDNA fusion complex.

51. The method of embodiment 50, wherein the promoter sequence is the T7 promoter sequence, wherein the protein translation initiation site is a Kozak sequence, the first peptide tag is a DYKDDDDK (SEQ ID NO. 1) tag, and wherein the second peptide tag is a Strep-tagII.

52. The method of embodiment 51, wherein preparation of a peptide/protein-mRNA fusion complex comprises:
(a) in vitro transcription of the double-stranded DNA to produce RNA,
(b) ligation of the RNA with a poly-dA DNA to produce ligated RNA/DNA,
(c) purification of ligated RNA
(d) in vitro translation of the ligated RNA/DNA to produce a peptide/protein-mRNA fusion complex, and
(e) purification of the peptide/protein-mRNA fusion complex.

53. The method of any one of embodiments 50-52, wherein cDNA synthesis on the peptide/protein-mRNA fusion complex with fluorescent labeling to generate a labeled peptide/protein-mRNA-cDNA fusion complex comprises:
reverse transcription of the peptide/protein-mRNA fusion using biotin-modified primer to produce a peptide/protein-mRNA-cDNA fusion, wherein the biotin modified primer anneals to both the poly-dA and constant region of the mRNA;
removal of unbound oligos; and
addition of fluorophore-conjugated streptavidin to bind the biotin on the cDNA and to generate a labeled peptide/protein-mRNA-cDNA fusion complex.

54. The method of any one of embodiments 49-53, wherein preparation of B cells labeled with mRNA-display epitope library comprises:
(iv) B Cell preparation; and
(v) B Cell staining and sorting.

55. The method of embodiment 54, wherein the B Cell preparation comprises: isolating B cells from one or more sources selected from the group consisting of humans, animals, fresh or properly frozen blood, and purified lymphocytes or tissues using selection kits, or staining by antibodies recognizing B cell marker(s).

56. The method of embodiment 54, wherein the B Cell staining comprises: staining of the fusion complex, or staining of the B cell marker, or both.

57. The method of embodiment 54, wherein the B Cell staining comprises: sorting of stained B cells by flow cytometry.

58. The method of any one of embodiments 49-57, wherein preparation of bar-coded beads comprises:
(vi) Hydrogel bead formation; and
(vii) Split-pool combinatorial barcoding of hydrogel beads.

59. The method of embodiment 58, wherein Hydrogel bead formation comprises: a continuous stream of aqueous phase that is emulsified into a stream of highly mono-disperse droplets that are collected and polymerized into Hydrogel beads.

60. The method of embodiment 58, wherein Hydrogel bead formation is carried out using a microfluidics device.

61. The method of embodiment 58, wherein Split-pool combinatorial barcoding of hydrogel beads comprises: stepwise enzymatic extension and hybridization reactions to add one or more barcoded primers to the beads.

62. The method of embodiment 61, wherein the Split-pool combinatorial barcoding of hydrogel beads is repeated four times to add four barcoded primers to the beads.

63. The method of embodiment 49, wherein Encapsulation of single-cell and single-bead into droplet comprises:
(viii) Encapsulation and cDNA synthesis;
(ix) Demulsification and DNA purification;
(x) cDNA amplification;
(xi) Epitope and B Cell Receptor sequence amplification;
(xii) Fragmentation
(xiii) End-repair/dA-tailing and adaptor ligation; and
(xiv) PCR amplification of the sequencing library.

64. The method of embodiment 49, wherein sequencing the library of encapsulated B cells comprises: Next Generation Sequencing of the sequencing library to provide an immunological profile.

65. The method of embodiment 64, wherein the immunological profile includes sequences of more than 1000 B cell receptor sequences, or sequences of more than 1000 B cell receptor epitopes, or both.

66. A method for identifying epitope-specific T cell receptor sequences for an antigen, comprising:
preparation of barcoded MHC tetramers in droplets;
T Cell staining and sorting;
Preparation of barcoded beads; and
Encapsulation of single-cell and single-bead into droplet and sequencing library preparation.

67. The method of embodiment 66, wherein preparation of barcoded MHC tetramers in droplets comprises:
(i) Preparation of a double-stranded DNA library from an antigen;
(ii) Preparation of fluorophore and oligo labeled streptavidin (FOS), or fluorophore labeled mono-avidin on branched DNA (FMbD)′; and
(iii) Assembly of barcoded MHC-tetramers/oligomers in droplets.

68. The method of embodiment 67, wherein the preparation of a double-stranded DNA library from an antigen comprises: self-circularization and isothermal amplification to form concatemers of multiple DNA variants.

69. The method of embodiment 67, wherein preparation of fluorophore and oligo labeled streptavidin (FOS) comprises: conjugating a DNA oligo to fluorophore-labeled streptavidin; and conjugation to biotinylated MHC.

70. The method of embodiment 67, wherein the Assembly of barcoded MHC-tetramers/oligomers in droplets comprises:
formation of barcoded MHC-tetramers or MHC-oligomers by in-droplet IVTT reaction; DNA-RNA hybridization; and
cDNA synthesis by reverse transcription.

71. The method of embodiment 70, wherein formation of barcoded MHC-tetramers or MHC-oligomers by in-droplet IVTT reaction comprises: forming droplets of DNA concatemer with an average occupancy of 1 concatemer in 5-10 droplets.

72. The method of any one of embodiments 1-71, wherein one or more steps is carried out using a microfluidic device.

73. The method of embodiment 72, wherein the formation of barcoded MHC-tetramers or MHC-oligomers occurs within each droplet in a microfluidic device.

74. The method of embodiment 66, wherein the T cell staining and sorting comprises:
(iv) T cell preparation; and
(v) T Cell staining and sorting.

75. The method of embodiment 74, wherein the T Cell preparation comprises: isolating T cells from one or more sources selected from the group consisting of humans, animals, fresh or properly frozen blood, and purified lymphocytes or tissues using selection kits, or staining by antibodies recognizing T cell marker(s).

76. The method of any one of embodiments 73-75, wherein the T Cell preparation comprises: attaching a barcode or other label to a multiplicity of T cells, wherein each T cell can be identified by the barcode or label.

77. The method of any one of embodiments 73-76, wherein the T Cell staining comprises: staining of the fluorescent MHCs tetramers/oligomers, or staining of the T cell marker, or both.

78. The method of any one of embodiments 73-77, wherein the T Cell sorting comprises: sorting of fluorescent stained MHC tetramers/oligomers by flow cytometry.

79. The method of embodiment 66, wherein preparation of bar-coded beads comprises:
(vi) hydrogel bead formation; and
(vii) split-pool combinatorial barcoding of hydrogel beads.

80. The method of embodiment 79, wherein hydrogel bead formation comprises: a continuous stream of aqueous phase that is emulsified into a stream of highly mono-disperse droplets that are collected and polymerized into hydrogel beads.

81. The method of embodiment 80, wherein hydrogel bead formation is carried out using a microfluidics device.

82. The method of embodiment 79, wherein split-pool combinatorial barcoding of hydrogel beads comprises: stepwise enzymatic extension and hybridization reactions to add one or more barcoded primers to the beads.

83. The method of embodiment 82, wherein the split-pool combinatorial barcoding of hydrogel beads is repeated four times to add four barcoded primers to the beads.

84. The method of embodiment 66, wherein encapsulation of single-cell and single-bead into droplet comprises:
(viii) Encapsulation and cDNA synthesis;
(ix) Demulsification and DNA purification; and
(x) cDNA amplification;
(xi) Epitope and T Cell Receptor sequence amplification;
(xii) Fragmentation
(xiii) End-repair/dA-tailing and adaptor ligation; and
(xiv) PCR amplification of the sequencing library.

85. The method of embodiment 84, wherein sequencing the library of encapsulated T cells comprises: Next Generation Sequencing of the sequencing library to provide an immunological profile.

86. The method of embodiment 85, wherein the immunological profile includes sequences of more than 1000 T cell receptor sequences, or sequences of more than 1000 T cell receptor epitopes, or both.

87. A method of making a vaccine against an antigen for a subject, comprising:
(a) identifying epitope-specific T cell receptor sequences for a multiplicity of epitopes within the antigen, wherein the epitope-specific T cell receptor sequences for each of the multiplicity of epitopes are determined according to any one of embodiments 66-86;
(b) determining which one or more of the multiplicity of epitopes for the antigen have the highest number of epitope-specific T cell receptor sequences; and
(c) preparing the vaccine for the subject including one or more of the epitopes determined in (b).

88. A method of making a vaccine against an antigen for a subject, comprising:
(a) identifying a multiplicity of antibody epitopes within the antigen by mRNA display,
wherein each of the multiplicity of the antibody epitopes are determined according to any one of embodiments 40-48; and
(b) preparing the vaccine for the subject including one or more of the epitopes determined in (a).

89. A method of making a vaccine against an antigen for a subject, comprising:
(a) identifying epitope-specific B cell receptor sequences for a multiplicity of epitopes within the antigen,
wherein the epitope-specific B cell receptor sequences for each of the multiplicity of epitopes are determined according to any one of embodiments 49-65
(b) determining which one or more of the multiplicity of epitopes for the antigen have the highest number of epitope-specific B cell receptor sequences; and
(c) preparing the vaccine for the subject including one or more of the epitopes determined in (b).

90. A vaccine prepared according to the method of any one of embodiments 87-89.

### BRIEF DESCRIPTION OF THE DRAWINGS

FIG. 1 is a cartoon representation showing the workflow of methods for using mRNA-display to profile the immune response based on production of immunoglobulin (antibody response) in a subject, including oligo synthesis or fragmentation of cDNA to prepare a library of fragments from cellular or pathogen proteome; in vitro transcription; linkage to poly-dA (or PolyA- puromycin) and puromycin; in vitro translation and reverse transcription to prepare nucleic acid-protein fusions. Nucleic acid-protein fusions are sequenced. Antibody-capture protein is coated onto plates and then incubated with body fluids containing immunoglobulin. Display is carried out on the nucleic acid-protein fusions using the immunoglobulin-bound plates, and target-binding nucleic acid-protein fusions are eluted, amplified by PCR and sequenced.

FIG. 2 is a schematic illustration of using mRNA-display and nano-droplet sequencing to determine the BCR receptor sequence and transcriptome at single-cell level. mRNA display library is bound to B cells and sorted; hydrogel beads formation is followed by bead barcoding (split and pool). Droplet formation is carried out by bead dissolving followed by cDNA synthesis and template switch; BCR and epitope sequence amplification; adaptor ligation and final amplification, and next generation sequencing.

FIG. 3 is a schematic illustration of using in-droplet tetramer loading and nano-droplet sequencing to determine TCR receptor sequence and transcriptome at single cell level. In-droplet peptide synthesis and tetramer loading are followed by T cell staining and sorting; hydrogel beads formation is followed by bead barcoding (split and pool). Droplet formation is carried out by bead dissolving followed by cDNA synthesis and template switch; TCR and epitope sequence amplification; adaptor ligation and final amplification, and next generation sequencing.

FIG. 4 shows a schematic illustration of oligo sequence composition of individual DNA variant in DNA library for mRNA-display, including (5′-3′) T7 promoter, 5′untranslated region, Kozak/ribosome binding sequence, 5′ protein binding tag, sub-library index tag 1, first linker, antigen sequence, second linker, sub-library index tag 2, and strep tag II.

FIG. 5 is a cartoon representation showing the workflow to prepare mRNA-display library. Oligo synthesis or fragmentation of cDNA is performed to prepare a library of fragments from cellular or pathogen proteome; addition of terminal FLAG and strep tags by PCR, addition of T7 promoter and Kozak by PCR; in vitro transcription; ligation to poly-dA-puromycin (or PolyA- puromycin) through aid of splint; digestion of poly-dA-puromycin (or PolyA- puromycin); RNA quality verification using Urea PAGE gel; in vitro translation and fusion, and reverse transcription.

FIG. 6 shows a schematic illustration of amplification with a barcoded primer, using the same sequence composition depicted in FIG. 4, showing position of barcode Forward and Reverse primers.

FIG. 7 is a cartoon representation of workflow of next-generation sequencing (NGS) library preparation, including barcoded DNA fragments, end-repair and dA tailing, Adaptor ligation, amplification with index primers, and completion of library ready for NGS.

FIG. 8 is a cartoon representation of workflow of barcoding hydrogel beads, including (1) in-droplet generation of acrylamide hydrogel beads; (2) linkage of 1^{st} barcode by isothermal amplification; (3) pooling and splitting beads to link 2^{nd} barcode, and (4) repeating step 3 to link the 3^{rd} barcode and 4^{th} barcode.

FIG. 9 is a cartoon representation of workflow of mRNA-display library preparation for B cell nano-droplet sequencing, including oligo synthesis or fragmentation of cDNA to prepare a library of fragments from cellular or pathogen proteome; addition of terminal FLAG and strep by PCR, addition of T7 promoter and Kozak by PCR; in vitro transcription; ligation to poly-dA-puromycin (or PolyA- puromycin) through aid of splint; digestion of poly-dA-puromycin and purification; RNA quality verification using Urea PAGE gel; in vitro translation and fusion, and reverse transcription using primers with biotin to provide an epitope-RNA-cDNA-Biotin complex.

FIG. 10 is a cartoon representation of cell barcoding and cDNA synthesis during nano-droplet sequencing, showing encapsulation, bead dissolving, cDNA synthesis annealing, and DNA extension.

FIG. 11A is a cartoon representation of workflow of NGS library preparation of nano-droplet sequencing, including release of droplets into bulk and purify DNA, amplify cDNA, amplify BCR and epitope sequence (1′ round), amplify BCR and epitope sequence (2^{nd} round), check size of the amplified DNA. FIG. 11B is a cartoon representation of workflow of NGS library preparation of nano-droplet sequencing, including DNA fragmentation (partial T7 exonuclease digest; 5′-3′ exonuclease activity), end-repair and dA tailing (target 200-1000 bp length); adapter ligation, final amplification, and library ready for NGS.

FIG. 12 is a cartoon representation of oligo sequence composition of individual DNA variant in DNA library for T cell epitope synthesis, including (5′-3′) T7 promoter, 5′untranslated region, Kozak/ribosome binding sequence (RBS), Factor Xa site, Epitope Sequence, Stop Codon, Constant region, Poly-dA, and T7 terminator.

(RBS: ribosome binding site; Antigen seq: Antigen sequence; TTS: transcription start site)

FIG. 13 is a cartoon representation of workflow for Isothermal amplification of oligos to form DNA concatemers.

FIGS. 14A-14B are cartoon representations of workflow for in-droplet peptide synthesis and MHC-tetramer loading.

FIG. 15 is a cartoon representation of workflow for in-droplet peptide synthesis and MHC-oligomer loading.

FIGS. 16A-16B show the distribution of IgG epitopes on SARS-CoV-2 virus. The antibodies were purified from COVID19 patients' sera at 1 month post symptom onset. FIG. 16A shows genome-wide distribution of epitopes on SARS-CoV-2 virus. The enrichment score is normalized against input. Enrichment score >1 indicates that the epitope is enriched. FIG. 16B shows distribution of epitopes on S protein of SARS-CoV-2. using the mRNA-display datasets.

FIGS. 17A-17D are graphs showing different epitopes of the SARS-COV-2 virus (NTD, RBD, S1/S2, and S2) over time (in-hospital, or 1 month (1 m), 4 months (4 m) or 6 months (6 m) post onset of symptoms), for each of IgG1 (FIG. 17A); IgG2 (FIG. 17B); IgG3 (FIG. 17C); and IgG4 (FIG. 17D), respectively.

FIGS. 18A-18E are bar graphs showing enrichment score for IgG against the SARS-COV-2 spike 384-432 protein over time (in-hospital, or 1 month (1 m), 4 months (4 m) or 6 months (6 m) post onset of symptoms), for each of total IgG (FIG. 18A); IgG1 (FIG. 18B); IgG2 (FIG. 18C); IgG3 (FIG. 18D); and IgG4 (FIG. 18E), respectively.

FIG. 19 is a graph of 45 autoantigens that have significantly higher enrichment in COVID19 patients versus pre-pandemic controls. Within the 45 autoantigens, 6 are associated with neurological disorders and 10 are associated with blood coagulation (See Tables 1 and 2).

FIGS. 20A-20D are bar graphs showing % input (0-30) over Fragment No. (1-10) for each of 10 ng Ab in BSA (FIG. 20A); 50 ng Ab in BSA (FIG. 20B); 10 ng Ab in serum (FIG. 20C); and 50 ng Ab in serum (FIG. 20D), respectively.

FIG. 21 is a graph showing temporal distribution of patient samples across different time points. Patients grouped by age are shown over Time from symptom onset (<2 weeks->27 weeks). Sample present is indicated by a shaded block.

FIG. 22 is a graph showing the Pearson correlation coefficient between technical replicates and the calculated enrichment score on the same serum sample. Pearson correlation coefficient (0-1.25) is shown over enrichment score.

FIGS. 23A-23B are graphs showing correlation of anti-CD3D auto-antibody test results (−0.1-0.5) (FIG. 23A); and anti-IL10RB auto-antibody test results (0-0.4) (FIG. 23B) showing ELISA OD450 over mRNA-display score (0-15), respectively.

FIGS. 24A-24X are graphs showing enrichment score for each of samples for pre-pandemics and COVID19, for each of S Protein (FIG. 24A); ORF8 (FIG. 24B); ORF9C (FIG. 24C); NSP2 (FIG. 24D); NSP6 (FIG. 24E); NSP8 (FIG. 24F); NSP9 (FIG. 24G); NSP10 (FIG. 24H); Exoribonuclease (FIG. 24I); ORF7A (FIG. 24J); N gene (FIG. 24K); RNA polymerase (FIG. 24L); E gene (FIG. 24M); NSP4 (FIG. 24N); NSP7 (FIG. 240); NSP1 (FIG. 24P); NSP3 (FIG. 24Q); Protease 3C (FIG. 24R); Helicase (FIG. 24S); Endoribonuclease (FIG. 24T); Methyltransferase (FIG. 24U); M gene (FIG. 24V); ORF7B (FIG. 24W); and ORF6 (FIG. 24X); respectively.

FIGS. 25A-25B are graphs showing time from symptom onset. FIG. 25A shows enrichment score for each of multiple genes; FIG. 25B shows enrichment score per epitope for each of multiple genes, respectively.

FIG. 26 is a graph showing Distribution of COVID19-specifically enriched peptides across the SARS-CoV-2 proteome for each of Pre-pandemics and COVID19 samples, respectively.

FIG. 27 is a bar graph showing S protein Distribution over Average enrichment score (1-32) for 24 amino acid peptide fragments of the S protein gene in (residues 0-1224, N-C term).

FIGS. 28A-28D are graphs showing enrichment score for each of samples for non-severe and severe COVID19 cases, for each of S Protein (FIG. 28A); residues 529-576 (FIG. 28B); residues 553-600 (FIG. 28C); and residues 817-864 (FIG. 28D), respectively.

FIG. 29 is a graph showing No. of fragments on S protein (0-15) over time for each of <2 weeks; 3-5 weeks; 6-9 weeks; 10-15 weeks; and 16-27 weeks, respectively.

FIGS. 30A-30D are graphs showing average (ave) enrichment score for each 24-residue fragment of S Protein for times ranging from <2 weeks (FIG. 30A); 10-15 weeks; (FIG. 30B); 3-5 weeks (FIG. 30C); and 16-27 weeks (FIG. 30D), respectively.

FIGS. 31A-31C are schematics and graphs showing correlation on antibody responses against different viruses between time points during COVID19 infection and recovery. Panels show Pearson correlation co-efficient between peptide enrichment scores of EBV (FIG. 31A); common cold HCoV (FIG. 31B); or SARS-CoV-2 (FIG. 31C), respectively, at multiple timepoints for one individual patient. SLISA enrichment scores of each single epitope on the sample collected at indicated time points (2 days, 2 weeks, 1 month, 4 months, respectively) for the same patient are shown below each Pearson correlation co-efficient graph.

FIGS. 32A-32D are graphs showing duration for each peptide of SARS-CoV-2 in patients with chronic diseases versus patients without chronic diseases. FIG. 32A is a dot plot showing −log 10-p-value over time (weeks) for each of Average (ave) chronic diseases patients and patients without chronic diseases. Each dot represents one peptide. Peptides in S protein were colored in grey and in square shape. Peptides with significantly differential duration (p<0.05, difference >2 weeks) were colored in gray and in triangle. FIGS. 32B-32D are graphs showing duration (weeks) of peptides with significantly differential duration in patients with chronic diseases and patients without chronic diseases, respectively, for each of S protein (672-720) FIG. 32B; N protein (240-288) FIG. 32C; and ORF1 (2062-2114) FIG. 32D, respectively. Each dot represents the duration for one patient.

FIGS. 33A-33D are graphs showing number of variants at each amino acid position (A-D) within S protein 649-696 peptide that can be bound by multiple time points of each patient. “033”, “045”, “104”, “105” represent identification numbers of patients. “A” represents 1-2 weeks, “B” represent 6-8 weeks, “C” represents 8-12 weeks and “D” represents 14-17 weeks. Each dot represents one amino acid position. FIG. 33A plots the data of patient 033; FIG. 33B plots the data of patient of 045; FIG. 33C plots the data of patient 104; and FIG. 33D plots the data of patient 105, respectively.

FIGS. 34A-34E are graphs showing SLISA-revealed antibody responses against SARS-CoV-2 peptide variants. Enrichment score of wildtype (triangle) and indicated variant (circle) peptides on the samples of patient 126 collected at indicated time points (4 days, 5 days, 9 days, 11 days, 1 month, 2 months, and 4 months). FIG. 34A plots the data for spike 501; FIG. 34B plots the data for spike 452; FIG. 34C plots the data for spike D138Y; FIG. 34D plots the data for spike D80A; FIG. 34E plots the data for spike K417N; and FIG. 34F plots N439K, respectively.

FIGS. 35A-35B are schematics and graphs showing SLISA-revealed antibody responses against auto-antigens in human sera. FIG. 35A shows Physical or functional interactions of COVID19 associated auto-antigens. The interactions were analyzed by String database with default setting. Three functional groups were circled by blue dash lines with group labels by the side. FIG. 35B is a Dot plot of pathway analysis of COVID19 associated auto-antigens. Each dot represents each individual pathway. Colors of the dots represent p-value. Sizes of the dots represent the number of genes enriched in the corresponding pathway. Pathway analysis was based on Kegg Human database shows SLISA-revealed differential antibody responses against auto-antigens in SLE, mononucleosis and COVID19 patients.

FIG. 36 is a graph showing enrichment score (0-15) for each of samples from COVID19; pre-pandemic; Mono; and SLE, respectively.

FIGS. 37A-37F are graphs showing Enrichment score over time points (4 days, 5 days, 9 days, 11 days, 1 month, 2 months, and 4 months) for each of Serpine1 (Patient C3); (FIG. 37A); Serpine1 (Patient C6) (FIG. 37B); Serpine1 (Patient K3) (FIG. 37C); ITGA2B (Patient C3) (FIG. 37D); ITGA2B (Patient C6) (FIG. 37E); and ITGA2B (Patient K3) (FIG. 37F), respectively.

FIGS. 38A-38F are flow cytometry plot graphs showing FSC over Anti-p-ERK for each of samples containing Anti-CD3+WT cells (FIG. 38A); Anti-CD3+CD3D KO cells (FIG. 38B); Serum 127+WT cells (FIG. 38C); Serum 127+CD3D KO cells (FIG. 38D); Serum 098+WT cells (FIG. 38E); and Serum 098+CD3D KO cells (FIG. 38F), respectively. FIG. 38G is an electron micrograph showing a gel stained for CD3D and GADPH in each of NC and KO samples, respectively. FIGS. 38H-38K are flow cytometry plot graphs showing FSC over Anti-p-ERK for samples containing Basic medium+patient serum from patient 111, stained for Anti-CD3D (FIG. 38H); and PMA/Iono (FIG. 38I), respectively, or samples containing Basic medium+FCS, stained for Anti-CD3D (FIG. 38J); and PMA/Iono (FIG. 38K), respectively.

### DETAILED DESCRIPTION OF THE INVENTION

### I. Definitions

The term “immunological profile” refers to a dataset including more than one nucleic acid sequence corresponding to an epitope of an antigen, or an antigen-binding immune receptor, or both. In the context of an immune response in an individual, an immunological profile can include multiple nucleic acid and optionally polypeptide sequences corresponding to antigenic epitopes recognized by immune receptors in the individual. Exemplary immune receptors include T cell receptors of T cells, B cell receptors of B cells, antigen binding portions of immunoglobulins, or other components of immune effecter cells. In some forms, an immunological profile includes the B cell receptor sequences specific for one or multiple antigens and T cell receptor sequences specific for one or multiple antigens of an individual, or both. In some forms, an immunological profile includes the B cell single cell transcriptome specific for one or multiple antigens of an individual or the T cell single cell transcriptome one or multiple antigens of an individual of an individual, or both.

The terms “enrich” and “enrichment” refer to an increase in the proportion of a component relative to other components present or originally present. In the context of nucleic acids, enrichment of nucleic acids in a sample refers to an increase in the proportion of the nucleic acids in the sample relative to other molecules in the sample. “Selective enrichment” is enrichment of particular components relative to other components of the same type. In the context of nucleic acid fragments, selective enrichment of a particular nucleic acid fragment refers to an increase in the proportion of the particular nucleic acid fragment in a sample relative to other nucleic acid fragments present or originally present in the sample. The measure of enrichment can be referred to in different ways. For example, enrichment can be stated as the percentage of all of the components that is made up by the enriched component. For example, particular nucleic acid fragments can be enriched in an enriched nucleic acid sample to at least 2-fold over the other nucleic acids in the sample.

The term “nucleic acid fragment” refers to a portion of a larger nucleic acid molecule. A “contiguous nucleic acid fragment” refers to a nucleic acid fragment that represents a single, continuous, contiguous sequence of the larger nucleic acid molecule. A “naturally occurring nucleic acid fragment” refers to a nucleic acid fragment that represents a single, continuous, contiguous sequence of a naturally occurring nucleic acid sequence.

The term “naturally occurring” refers to a molecule that has the same structure or sequence as the corresponding molecule as it exists in nature. A naturally occurring molecule or sequence can still be considered naturally occurring when it is coupled to or incorporated into another molecule or sequence.

The term “nucleic acid sample” refers to a composition, such as a solution, that contains or is suspected of containing nucleic acid molecules.

The term “nucleotide” refers to a molecule that contains a base moiety, a sugar moiety, and a phosphate moiety. Nucleotides can be linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The terms “oligonucleotide” or a “polynucleotide” are synthetic or isolated nucleic acid polymers including a plurality of nucleotide subunits.

The terms “peptide” and “polypeptide” refer to a class of compounds composed of amino acids chemically bound together. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids and small and large peptides, including polypeptides.

### II. Methods for Generating Immunological Profiles

Methods for generating immunological profiles have been designed and/or established. The methods exploit the discovery that dynamics of adaptive immune responses against infection or self-antigens can be reflected by antibody binding activity and B/T cell receptor sequences binding to a set of defined epitopes. The methods provide immunological profiles for an immune response in an individual and provide information about pathogenesis, potential therapeutic targets, and guidance on vaccine development. The methods are embodied by a technology platform, named Immunological Profiling using Displays (IPD) that enables genome-scale determination of epitope-specific antibody and B- and T- cell receptor sequences and cellular activities. The methods encompass three inter-linked components to determine the specificity of (1) antibodies, (2) B cells and (3) T cells at single epitope resolution at the genomic scale using mRNA-display and its variation. The information collected by the three components can be integrated within a comprehensive immunological profile of the activity and specificity of adaptive immunity for a particular subject (human or animal). Changes of the profiles at different time points accurately and comprehensively reflect the immunological responses in a subject. The high-resolution profiles of immune responses at genomic scale will enable precise diagnosis.

The methods employ modified RNA display and variations of the display to establish the linkage between epitopes with the corresponding B/T cell receptors. Instead of affinity matured protein binders, the methods use mRNA display to generate hundreds of thousands or millions of epitopes (peptides/proteins), each with a unique RNA/DNA barcode attached to it, allowing identification of epitope-specific antibodies or B/T cell receptors. The methods also include droplet display (nano-droplet formation with microfluidics), without using puromycin, to enable the profile of B/T cell receptors.

### A. Immunogens/Antigens for Generating an Immune Response

The methods characterize an immune response to an antigen (or a set of antigens) in an individual, including generating an immunological profile for an immune response in the individual to the antigen (or a set of antigens). In some forms, the immunological profiles include sequence information about antibody epitopes identified by mRNA display. In some forms, the immunological profiles include sequences of epitope-specific B cells, including but not limited to BCR sequences. In some forms, the immunological profiles include sequences of epitope-specific T cells, including but not limited to TCR sequences.

Typically, the immune response mounted in a subject is against one or more antigens or antigenic epitopes from a species, such as pathogens (e.g., viruses, bacteria, fungi etc.), mammalian cells (e.g., for autoantigens or tumor antigens), or other species (e.g., allergens, vaccines). In some forms, the antigens or antigenic epitopes are derived from a virus, bacterium, parasite, plant, protozoan, fungus, tissue or transformed cell such as a cancer or leukemic cell and immunogenic component thereof.

In preferred forms, the antigenic epitopes are derived from a viral antigen. A viral antigen can be isolated from any virus. In an exemplary form, the antigen is a natural viral capsid structure, or one or more components from an inactivated or “killed” virus. An exemplary inactivated virus antigen is a haemaglutinin and/or neuraminidase protein from a split influenza virus. In other forms, the antigenic epitopes are derived from a bacterial antigen. Bacterial antigens can originate from any bacteria. In some forms the antigen is a parasite antigen. In some forms, the antigenic epitopes are derived from an allergen or environmental antigen. Exemplary allergens and environmental antigens, include but are not limited to, an antigen derived from naturally occurring allergens such as pollen allergens (tree-, herb, weed-, and grass pollen allergens), insect allergens (inhalant, saliva, and venom allergens), animal hair and dandruff allergens, and food allergens. In some forms, the antigenic epitopes are derived from a self-antigen such as in immune tolerance applications for auto-immune or related disorders such as lupus, multiple sclerosis. In some forms, the antigenic epitopes are derived from a tumor antigen. Exemplary tumor antigens include a tumor-associated or tumor-specific antigen.

### 1. Viral Antigens

In preferred forms, the antigen is a viral antigen isolated from a virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenza virus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens can be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

Exemplary viral antigens include influenza virus hemagglutinin (HA) (Genbank accession No. JO2132; Air, 1981, Proc. Natl. Acad. Sci. USA 78:7639-7643; Newton et al., 1983, Virology 128:495-501), influenza virus neuraminidase (NA), PB1, PB2, PA, NP, M1, M2, NS1, NS2)) of Influenza virus; E1A, E1B, E2, E3, E4, E5, L1, L2, L3, L4, L5 of Adenovirus; Pneumonoviridae (e.g., pneumovirus, human respiratory syncytial virus): Papovaviridae (polyomavirus and papillomavirus): E1, E2, E3, E4, E5a, E5b, E6, E7, E8, L1, L2; Human respiratory syncytial virus: human respiratory syncytial virus: G glycoprotein (Genbank accession no. Z33429; Garcia et al., 1994, J. Virol.; Collins et al., 1984, Proc. Natl. Acad. Sci. USA 81:7683), RSV-viral proteins, e.g., RSV F glycoprotein; Dengue virus: core protein, matrix protein or other protein of Dengue virus (Genbank accession no. M19197; Hahn et al., 1988, Virology 162:167-180); Measles: measles virus hemagglutinin (Genbank accession no. M81899; Rota et al., 1992, Virology 188:135-142); Herpesviridae (e.g., herpes simplex virus 1, herpes simplex virus 2, herpes simplex virus 5, and herpes simplex virus 6: herpes simplex virus type 2 glycoprotein gB (Genbank accession no. M14923; Bzik et al., 1986, Virology 155:322-333), gB, gC, gD, and gE, HIV (GP-120, p17, GP-160, gag, pol, qp41, gpl20, vif, tat, rev, nef, vpr, vpu, vpx antigens), ribonucleotide reductase, a −TIF, ICP4, ICP8, 1CP35, LAT-related proteins, gB, gC, gD, gE, gH, gI, gJ, and dD antigens; Lentivirus (e.g., human immunodeficiency virus 1 and human immunodeficiency virus 2): envelope glycoproteins of HIV I (Putney et al., 1986, Science 234:1392-1395); Picornaviridae (e.g., enterovirus, rhinovirus, hepatovirus (e.g., human hepatitis A virus); Cardiovirus; Apthovirus; Reoviridae (orthoreovirus, orbivirus, rotavirus, cypovirus, fijivirus, phytoreovirus, and oryzavirus), Retroviridae (mammalian type B retroviruses, mammalian type C retroviruses, avian type C retroviruses, type D retrovirus group, BLV-HTLV retroviruses); spumavirus, flaviviridae (e.g., hepatitis C virus), hepadnaviridae (e.g., hepatitis B virus), togaviridae (e.g., alphavirus (e.g., sindbis virus) and rubivirus (e.g., rubella virus), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus); Poliovirus: I VP1 (Emini et al., 1983, Nature 304:699); Hepatitis B virus: hepatitis B surface antigen (Itoh et al., 1986, Nature 308:19; Neurath et al., 1986, Vaccine 4:34), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651-693; Tiollais et al., 1985, Nature 317:489-495), hepatitis (Hep B Surface Antigen (gp27S, gp36S, gp42S, p22c, pol, x)). Additional viruses include Ebola, Marburg, Rabies, Hanta virus infection, West Nile virus, SARS-like Coronaviruses, Varicella-zoster virus, Epstein-Barr virus, Alpha virus, St. Louis encephalitis. Adenovirdiae (mastadenovirus and aviadenovirus), Leviviridae (levivirus, enterobacteria phase MS2, allolevirus), Poxyiridae (e.g., chordopoxyirinae, parapoxvirus, avipoxvirus, capripoxvirus, leporipoxvirus, suipoxvirus, molluscipoxvirus, and entomopoxyirinae), Papovaviridae (polyomavirus and papillomavirus); Paramyxoviridae (paramyxovirus, parainfluenza virus 1), Mobillivirus (measles virus), Rubulavirus (mumps virus), metapneumovirus (e.g., avian pneumovirus and human metapneumovirus); Pseudorabies: pseudorabies virus g50 (gpD), pseudorabies virus II (gpB), pseudorabies virus gIII (gpC), pseudorabies virus glycoprotein H, pseudorabies virus glycoprotein E; transmissible gastroenteritis including transmissible gastroenteritis glycoprotein 195, transmissible gastroenteritis matrix protein; Newcastle virus including Newcastle disease virus hemagglutinin-neuraminidase; infectious laryngotracheitis virus including viral antigens such as infectious laryngotracheitis virus glycoprotein G or glycoprotein 1; La Crosse virus including viral antigen such as a glycoprotein of La Crosse virus (Gonzales-Scarano et al., 1982, Virology 120:42).

Exemplary swine viruses include swine rotavirus glycoprotein 38, swine parvovirus capsid protein, Serpulina hydodysenteriae protective antigen, bovine viral diarrhea glycoprotein 55, neonatal calf diarrhea virus (Matsuno and Inouye, 1983, Infection and Immunity 39:155), hog cholera virus, African swine fever virus, swine influenza including antigens such as swine flu hemagglutinin and swine flu neuraminidase.

Exemplary equine viruses include equine influenza virus or equine herpesvirus: equine influenza virus type A/Alaska 91 neuraminidase, equine influenza virus type A/Miami 63 neuraminidase, equine influenza virus type A/Kentucky 81 neuraminidase, equine herpesvirus type 1 glycoprotein B, and equine herpesvirus type 1 glycoprotein D, Venezuelan equine encephalomyelitis virus (Mathews and Roehrig, 1982, J. Immunol. 129:2763).

Exemplary cattle viruses include bovine respiratory syncytial virus or bovine parainfluenza virus: bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and bovine parainfluenza virus type 3 hemagglutinin neuraminidase), bovine viral diarrhea virus glycoprotein 48 or glycoprotein 53, infectious bovine rhinotracheitis virus: infectious bovine rhinotracheitis virus glycoprotein E or glycoprotein G, foot and mouth disease virus, punta toro virus (Dalrymple et al., 1981, in Replication of Negative Strand Viruses, Bishop and Compans (eds.), Elsevier, N.Y., p. 167).

i. Influenza Antigens

In some forms, the antigen is derived from an influenza virus. Influenza Virus antigens can be derived from a particular influenza clade or strain, or can be synthetic antigens, designed to correspond with highly conserved epitopes amongst multiple different influenza virus strains.

There are four types of influenza viruses: A, B, C and D. Human influenza A and B viruses cause seasonal epidemics of disease. Influenza A viruses are the only influenza viruses known to cause flu pandemics, i.e., global epidemics of flu disease. Influenza type C infections generally cause mild illness and are not thought to cause human flu epidemics Influenza D viruses primarily affect cattle and are not known to infect or cause illness in people (see w.w.w.cdc.gov/flu/about/viruses/types.htm).

The influenza A virion is studded with glycoprotein spikes of hemagglutinin (HA) and neuraminidase (NA), in a ratio of approximately four to one, projecting from a host cell-derived lipid membrane. A smaller number of matrix (M2) ion channels traverse the lipid envelope, with an M2:HA ratio on the order of one M2 channel per 101-102 HA molecules. The envelope and its three integral membrane proteins HA, NA, and M2 overlay a matrix of M1 protein, which encloses the virion core. Internal to the M1 matrix are found the nuclear export protein (NEP; also called nonstructural protein 2, NS2) and the ribonucleoprotein (RNP) complex, which includes of the viral RNA segments coated with nucleoprotein (NP) and the heterotrimeric RNA-dependent RNA polymerase, composed of two “polymerase basic” and one “polymerase acidic” subunits (PB1, PB2, and PA). The organization of the influenza B virion is similar, with four envelope proteins: HA, NA, and, instead of M2, NB and BM2. Therefore, in some forms, the antigen is derived from one or more of the HA, NA, M2, NS2, NB, PB1, PB2, PA or NP genes of any influenza A or B virus.

Influenza A viruses are divided into subtypes based on hemagglutinin (H) and neuraminidase (N) proteins on the surface of the virus. There are 18 different hemagglutinin subtypes and 11 different neuraminidase subtypes (H1 through H18, and N1 through N11, respectively). Therefore, in some forms, the antigen is derived from the HA gene of an influenza virus influenza from any one or more of the H1 through H18 subtypes. In other forms, the antigen is derived from the NA gene of an influenza virus from any one or more of the N1 through N11 subtypes. While there are potentially 198 different influenza A subtype combinations, only 131 subtypes have been detected in nature. Current subtypes of influenza A viruses that routinely circulate in people include: A(H1N1) and A(H3N2). Therefore, in some forms, the antigen is derived from an A(H1N1) influenza virus, or an A(H3N2) influenza virus.

Influenza A viruses are further classified into multiple subtypes (e.g., H1N1, or H3N2), while influenza B viruses are classified into one of two lineages: B/Yamagata and B/Victoria. Both influenza A and B viruses can be further classified into specific clades and sub-clades. Clades and sub-clades can be alternatively called “groups” and “sub-groups,” respectively. An influenza clade or group is a further subdivision of influenza viruses (beyond subtypes or lineages) based on the similarity of their HA gene sequences. Clades and subclades are shown on phylogenetic trees as groups of viruses that usually have similar genetic changes (i.e., nucleotide or amino acid changes) and have a single common ancestor represented as a node in the tree. Clades and sub-clades that are genetically different from others are not necessarily antigenically different (i.e., viruses from a specific clade or sub-clade may not have changes that impact host immunity in comparison to other clades or sub-clades).

Currently circulating influenza A(H1N1) viruses are related to the pandemic 2009 H1N1 virus that emerged in spring of 2009 and caused a flu pandemic (See w.w.w.cdc.gov/flu/about/viruses/types.htm). This virus is known as “A(H1N1)pdm09 virus,” or “2009 H1N1,” and continued to circulate seasonally from 2009 to 2021. These H1N1 viruses have undergone relatively small genetic changes and changes to their antigenic properties over time. Of the influenza viruses that circulate and cause human disease, influenza A(H3N2) viruses tend to change more rapidly, both genetically and antigenically and have formed many separate, genetically different clades that continue to co-circulate. Therefore, in some forms, the antigen is derived from all currently circulating H1N1 influenza viruses.

In some forms, the antigen is derived from all currently circulating H3N2 influenza viruses. In preferred forms, the antigen is derived from all currently circulating H1N1 influenza viruses and H3N2 influenza viruses. In some forms, the antigen is derived from an Influenza A virus NP gene, or an Influenza A virus NP gene expression product.

Influenza B viruses are classified into two lineages: B/Yamagata and B/Victoria. Influenza B viruses are further classified into specific clades and sub-clades. Influenza B viruses change more slowly in terms of genetic and antigenic properties than influenza A viruses. Surveillance data from recent years shows co-circulation of influenza B viruses from both lineages in the United States and around the world with. Therefore, in some forms, the antigen is derived from influenza B viruses. In some forms, the antigen is derived from all currently circulating influenza B viruses. In some forms, the antigen is derived from an Influenza B virus NP gene, or an Influenza B virus NP gene expression product.

In some forms, the antigen is derived from B/Yamagata and B/Victoria influenza viruses. In other forms, the antigen is derived from one or more H1N1 influenza virus, and one or more influenza B virus. In other forms, the antigen is derived from one or more H3N2 influenza virus, and to one or more influenza B virus. In other forms, the antigen is derived from one or more H1N1 influenza virus, to one or more H3N2 influenza virus, and to one or more influenza B virus.

Exemplary antigens include influenza virus hemagglutinin (Genbank accession No. JO2132; Air, 1981, Proc. Natl. Acad. Sci. USA 78:7639-7643; Newton et al., 1983, Virology 128:495-501), influenza virus neuraminidase, PB1, PB2, PA, NP, M₁, M₂, NS₁, NS2)) of Influenza virus; swine influenza including antigens such as swine flu hemagglutinin and swine flu neuraminidase. Exemplary equine viruses include equine influenza virus or equine herpesvirus: equine influenza virus type A/Alaska 91 neuraminidase, equine influenza virus type A/Miami 63 neuraminidase, equine influenza virus type A/Kentucky 81 neuraminidase. Exemplary cattle viruses include bovine parainfluenza virus type 3 fusion protein, and bovine parainfluenza virus type 3 hemagglutinin neuraminidase).

ii. Coronavirus Antigens

In some forms, the antigen is derived from one or more coronaviruses. The coronaviruses (order Nidovirales, family Coronaviridae, and genus Coronavirus) are a diverse group of large, enveloped, positive-stranded RNA viruses that cause respiratory and enteric diseases in humans and other animals.

Coronaviruses typically have narrow host range and can cause severe disease in many animals, and several viruses, including infectious bronchitis virus, feline infectious peritonitis virus, and transmissible gastroenteritis virus, are significant veterinary pathogens. Human coronaviruses (HCoVs) are found in both group 1 (HCoV-229E) and group 2 (HCoV-OC43) and are historically responsible for ˜30% of mild upper respiratory tract illnesses.

At ˜30,000 nucleotides, their genome is the largest found in any of the RNA viruses. There are three groups of coronaviruses; groups 1 and 2 contain mammalian viruses, while group 3 contains only avian viruses. Within each group, coronaviruses are classified into distinct species by host range, antigenic relationships, and genomic organization. The genomic organization is typical of coronaviruses, with the characteristic gene order (5′-replicase [rep], spike [S], envelope [E], membrane [M], nucleocapsid [N]-3′) and short untranslated regions at both termini. The SARS rep gene, which includes approximately two-thirds of the genome, encodes two polyproteins (encoded by ORF1a and ORF1b) that undergo co-translational proteolytic processing. There are four open reading frames (ORFs) downstream of rep that are predicted to encode the structural proteins, S, E, M, and N, which are common to all known coronaviruses.

iii. SARS-CoV-2 Antigens

In some forms, the antigen is an antigen from a severe acute respiratory syndrome coronavirus-2 (SARS-CoV-2) betacoronavirus of the subgenus Sarbecovirus.

SARS-CoV-2 is a novel coronavirus that emerged in December 2019 and quickly caused a global pandemic. As of late June 2021, the virus has already caused more than 180 million infections and nearly four million deaths worldwide.

SARS-CoV-2 viruses share approximately 79% genome sequence identity with the SARS-CoV virus identified in 2003. An exemplary nucleic acid sequence for the SARS-CoV-2 ORF1a/b gene is set forth in GenBank accession number MN908947.3. The genome organization of SARS-CoV-2 viruses is shared with other betacoronaviruses; six functional open reading frames (ORFs) are arranged in order from 5′ to 3′: replicase (ORF1a/ORF1b), spike (S), envelope (E), membrane (M) and nucleocapsid (N). In addition, seven putative ORFs encoding accessory proteins are interspersed between the structural genes.

iv. Antibodies to SARS-CoV-2

Antibodies developed in patients infected with seasonal coronaviruses decline over time and may persist for as short as three months. In some cases of two other human coronaviruses that cause severe diseases, Severe Acute Respiratory Syndrome Coronavirus (SARS-CoV) and MERS, antibodies can still be detected years after infection. Patients infected with SARS-CoV-2 seroconvert within three weeks of symptom onset, developing antibodies most notably the receptor-binding domain (RBD) on the Spike (S) protein as well as other antigenic viral proteins. While some studies show that the early presence of antibodies against the virus is crucial for the clearance of viral RNA, other studies have indicated that high antibody titers are associated with disease severity and even mortality. Moreover, while antibody titer declines during recovery after the initial peak, some studies report significant neutralizing antibody titers two months post symptom onset, while others report near-baseline or undetectable titers as time progresses. Antibody response against individual viral proteins or epitopes across the viral genome may vary dramatically from each other. While these studies shed light on the temporal dynamics of antibody response during SARS-CoV-2 infection, they lack the identification of epitopes at high resolution that these antibodies may target. Recent research using protein-protein interaction assays, such as phage display, peptide microarray, and ELISA, have mapped epitopes across the SARS-CoV-2 proteome.

v. SARS-CoV-2 Autoantibodies

While patients' antibodies protect against infection, they may also contribute significantly to the pathogenicity of coronavirus disease (COVID-19). In particular, emerging evidence points to the role of autoantibodies in the immunopathology of COVID19. Autoimmune symptoms related to immune thrombocytopenic purpura, antiphospholipid syndrome, and autoimmune-like neurological diseases have been reported and multiple studies report the presence of autoantibodies against immunomodulatory proteins, anti-nuclear proteins, proteins involved in platelet regulation and coagulation in COVID19 patients.

### 2. Other Antigens

In some forms, the antigens are non-viral antigens. Exemplary non-viral antigens include, but are not limited to bacterial, protozoan, fungal, helminth and environmental antigens. Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

In some forms, the antigenic or immunogenic protein fragment or epitope is derived from a pathogenic bacteria such as Anthrax; Chlamydia: Chlamydia protease-like activity factor (CPAF), major outer membrane protein (MOMP); Mycobacteria; Legioniella: Legionella peptidoglycan-associated lipoprotein (PAL), mip, flagella, OmpS, hsp60, major secretory protein (MSP); Diphtheria: diphtheria toxin (Audibert et al., 1981, Nature 289:543); Streptococcus 24M epitope (Beachey, 1985, Adv. Exp. Med. Biol. 185:193); Gonococcus: gonococcal pilin (Rothbard and Schoolnik, 1985, Adv. Exp. Med. Biol. 185:247); Mycoplasm: Mycoplasma hyopneumoniae; Mycobacterium tuberculosis: M. tuberculosis antigen 85A, 85B, MPT51, PPE44, mycobacterial 65-kDa heat shock protein (DNA-hsp65), 6-kDa early secretary antigenic target (ESAT-6); Salmonella typhi; Bacillus anthracis B. anthracis protective antigen (PA); Yersinia perstis: Y. pestis low calcium response protein V (LcrV), F1 and F1-V fusion protein; Francisella tularensis; Rickettsia typhi; Treponema pallidum; Salmonella: SpaO and H1a, outer membrane proteins (OMPs); and Pseudomonas: P. aeruginosa OMPs, PcrV, OprF, OprI, PilA and mutated ToxA.

In some forms, the antigenic or immunogenic protein fragment or epitope is derived from a pathogenic fungus, including, but not limited to, Coccidioides immitis: Coccidioides Ag2/Pra106, Prp2, phospholipase (P1b), alpha-mannosidase (Amn1), aspartyl protease, Gell; Blastomyces dermatitidis: Blastomyces dermatitidis surface adhesin WI-1; Cryptococcus neoformans: Cryptococcus neoformans GXM and its Peptide mimotopes, and mannoproteins, Cryptosporidiums surface proteins gp15 and gp40, Cp23 antigen, p23; Candida spp. including C. albicans. C. glabrata. C. parapsilosis. C. dubliniensis. C. krusei. and others; Aspergillus species: Aspergillus Asp f 16, Asp f 2, Der p 1, and Fel d 1, rodlet A, PEP2, Aspergillus HSP90, 90-kDa catalase.

In some forms, the antigenic or immunogenic protein fragment or epitope is derived from a pathogenic protozoan. Exemplary protozoa or protozoan antigens include: Plasmodium falciparum, Plasmodium vivax, Plasmodium ovale, Plasmodium malariae, Plasmodium apical membrane antigen 1 (AMA1), 25-kDa sexual-stage protein (Pfs25), erythrocyte membrane protein 1 (PfEMP1) circumsporozoite protein (CSP), Merozoite Surface Protein-1 (MSP1); Leishmania species: Leishmania cysteine proteinase type III (CPC) Trypanosome species (African and American): T. pallidum outer membrane lipoproteins, Trypanosome beta-tubulin (STIB 806), microtubule-associate protein (MAP p15), cysteine proteases (CPs) Cryptosporidiums; isospora species; Naegleria fowleri; Acanthamoeba species; Balamuthia mandrillaris; Toxoplasma gondii, or Pneumocystis carinii: Pneumocystis carinii major surface glycoprotein (MSG), p55 antigen; Babesia Schistosomiasis: Schistosomiasis mansoni Sm14, 21.7 and SmFim antigen, Tegument Protein Sm29, 26 kDa GST, Schistosoma japonicum, SjCTPI, SjC23, Sj22.7, or SjGST-32, Toxoplasmosis: Gondii surface antigen 1 (TgSAG1), protease inhibitor-1 (TgPI-1), surface-associated proteins MIC2, MIC3, ROP2, GRAi-GRA7.

In some forms, the antigen is a cancer antigen or a nucleic acid or vector thereof encoding a cancer antigen. A cancer antigen is an antigen that is typically expressed preferentially by cancer cells (i.e., it is expressed at higher levels in cancer cells than on non-cancer cells; cancer-associated antigen) and in some instances it is expressed solely by cancer cells (cancer-specific antigen). Cancer antigen may be expressed within a cancer cell or on the surface of the cancer cell. Exemplary cancer antigens include tumor-associated antigens (TAAs), tumor specific antigens (TSAs), tissue-specific antigens, viral tumor antigens, cellular oncogene proteins, and/or tumor-associated differentiation antigens. These antigens can serve as targets for the host immune system and elicit responses which result in tumor destruction. (1990) J. Biol. Response Mod. 9:499 511.

### 3. Vaccines

In some forms, the antigens are any approved vaccines that are designed to elicit an immune response to protect against infection with or disease caused by a particular pathogen. Vaccines for use in the compositions include but are not limited to whole-pathogen vaccines such as inactivated viruses, live-attenuated viruses, and chimeric vaccine; subunit vaccines such as protein subunit vaccines, peptide vaccines, virus-like particles (VLPs), and recombinant proteins; and nucleic acid-based vaccines such as DNA plasmid vaccines, mRNA vaccines, and recombinant vector vaccines utilizing viral expression vectors. Exemplary vaccines include Adenovirus Type 4 and Type 7 Vaccine, ERVEBO® (Ebola Zaire Vaccine, Live), DENGVAXIA® (Dengue Tetravalent Vaccine, Live), DAPTACEL® (Diphtheria and Tetanus Toxoids and Acellular Pertussis Vaccine), M-M-R II® (Measles, Mumps, and Rubella Virus Vaccine Live), TRUMENBA® (Meningococcal Group B Vaccine), POLIOVAX® (Poliovirus Vaccine Inactivated), IMOVAX® (Rabies Vaccine), RABAVERT® (Rabies Vaccine), ROTARIX® (Rotavirus Vaccine, Live), JYNNEOS® (Smallpox and Monkeypox Vaccine, Live), TYPHIM Vi® (Typhoid Vi Polysaccharide Vaccine), and YF-VAX® (Yellow Fever Vaccine). Exemplary COVID-19 vaccines include Pfizer-BioNTech COVID-19 vaccine, Moderna COVID-19 vaccine, Oxford/AstraZeneca COVID-19 vaccine, Russia's Sputnik V COVID-19 vaccine, and Chinese Sinopharm COVID-19 vaccine.

i. Influenza Vaccines

A preferred vaccine for use in the compositions is an influenza vaccine, such as a tetravalent seasonal influenza vaccine including an equal amount of each of 4 different influenza strains. The formulation of current seasonal influenza vaccines typically contains inactivated split-virion from two influenza A strains (H1N1 and H3N2) and two influenza B strains. For example, the vaccine recommended by World Health Organization for the 2017-2018 season for the northern hemisphere includes the following four influenza virus strains, wherein the hemagglutinin weight ratio is 1:1:1:1 (A/H1N1: A/H3N2: B: B): 15 micrograms HA—A/Michigan/45/2015 (H1N1)pdm09-like virus; 15 micrograms HA—A/Hong Kong/4801/2014 (H3N2) -like virus; 15 micrograms HA—B/Phuket/3073/2013-like virus from B/Yamagata lineage; and 15 micrograms HA—B/Brisbane/60/2008-like virus from B/Victoria lineage.

B. Identification of Antibody Epitopes by mRNA Display

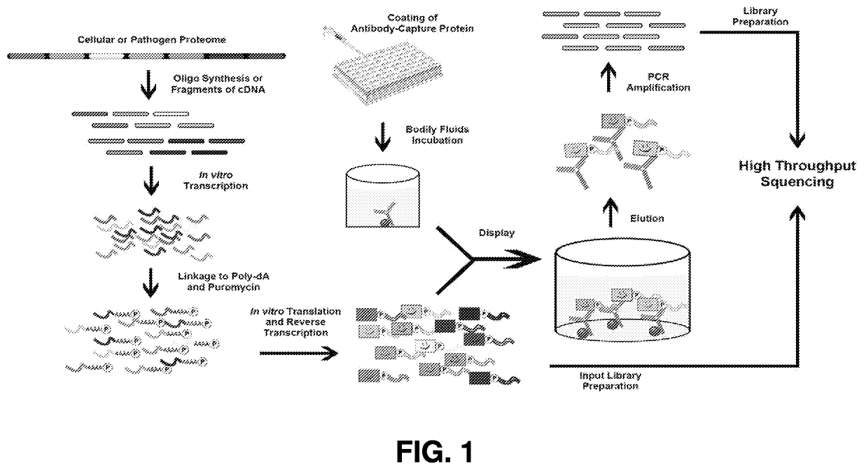

Using mRNA display, barcoded peptides/proteins can be derived from DNAs chemically synthesized or fragmented from cellular/viral/bacterial/fugal cDNA. A small portion of these epitopes may be recognized and enriched by immobilized antibodies. These epitopes can be identified by sequencing their barcodes. The steps are outlined as follows (FIG. 1).

In some forms, methods for identifying antibody epitopes by mRNA display include two steps, including (1) preparation of an mRNA-display epitope library and (2) immuno-capture of mRNA-display epitope library.

1. Preparation of an mRNA-Display Epitope Library

In some forms, methods for preparation of an mRNA-display epitope library include multiple steps, including (i) preparation of DNA library; (ii) preparation of peptide/protein-mRNA fusion complex; and (iii) cDNA synthesis to generate peptide/protein-mRNA-cDNA fusion complex.

i. Preparation of DNA Library

In some forms, methods for identifying antibody epitopes by mRNA display include a step of nucleic acid library preparation. Typically, a nucleic acid display library should include wild-type or mutant epitopes (peptides/domains) derived from a particular species, such as pathogens (e.g., viruses, bacteria, fungi etc.), mammalian cells (e.g., for autoantigens or tumor antigens), or other species (e.g., allergens).

Typically, DNA library preparation includes pooling multiple nucleic acids encoding the target epitopes. The nucleic acid encoding the target epitopes can be synthesized as a pool by one or more methods. Exemplary methods include on-chip DNA synthesis technologies, synthesis of regular oligo containing mutant cassettes, and fragmented from genomic or cDNAs. In some forms the pool contains as many as hundreds, thousands, tens of thousands, hundreds of thousands, millions, or tens of millions of DNA fragments. Typically, the upper limit of nucleic acid fragments is determined by oligo synthesis capacity, or by the availability of genomic/cDNA libraries.

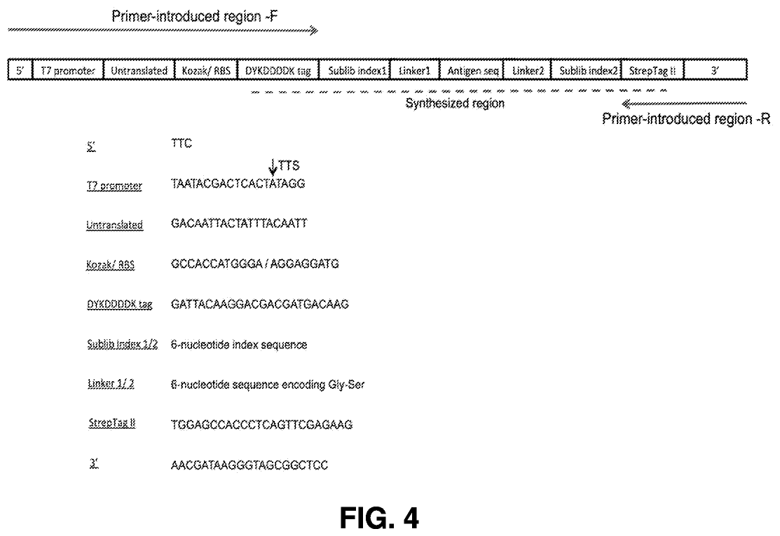

In some forms, the methods include one or more systems or methods for the design of the oligo pool. For example, in some forms, synthetic DNA fragments are linked with a promoter sequence (e.g., T7 promoter sequence), a nucleic acid motif that functions as the protein translation initiation site (e.g., Kozak consensus sequence or Kozak sequence), and sequence encoding a first peptide tag (e.g., DYKDDDDK (SEQ ID NO. 1) tag) at the 5′ end; and sequence encoding a second peptide tag (e.g., Strep-tagII) at their 3′ end, by Polymerase Chain Reaction (PCR). An exemplary oligo sequence is set forth in FIG. 4. The methods then employ the resulted double-stranded DNA for generating RNA in the following steps.

ii. Preparation of Peptide/Protein-mRNA Fusion Complex

In some forms, methods for identifying antibody epitopes by mRNA display include a step of preparation of peptide/protein-mRNA fusion complex. An exemplary scheme for preparation of peptide/protein-mRNA fusion complex is illustrated in FIG. 5. The following protocols (steps a-e) illustrate how the described methods for preparation of peptide/protein-mRNA fusion complex can be incorporated into the workflow for identification of antibody epitopes by mRNA display:

### (a) In Vitro Transcription

Typically, the methods include in vitro transcription of the pool of double-stranded DNA generated in the previous step into RNA. In some forms in vitro transcription is carried out by T7 RNA polymerase.

An exemplary in vitro transcription reaction set up (Using NEB T7 RNA polymerase) is as follows:

| | | |
|---|---|---|
| 10 × buffer | 2 | μL |
| 25 uM dNTP | 0.8 | μL |
| 100 uM DTT | 1 | uL |
| T7 Polymerase | 2 | μL |
| RNase inhibitor, Murine (40 units/ul) | 0.5 | μL |
| Template DNA | 200 | ng |

| | |
|---|---|
| Nuclease-free water | Up to 20 μL |

The reaction should be incubated at 37° C. for 2 hours, followed by RNA purification using an RNA clean-up kit, such as Monarch® RNA Cleanup Kit (NEB).

### (b) Ligation

The methods ligate the purified RNA with a poly-dA DNA oligo fused with a puromycin at the 3′ end, with assistance of a splint sequence.

An exemplary RNA-poly-dA-puromycin ligation reaction is as follows:

Poly-dA-puromycin oligo:
(SEQ ID NO. 2)
Phospho-AAAAAAAAAAAAAAAAAAAAA-spacer9-spacer9-
spacer9-ACC-puromycin.
Splint oligo:
(SEQ ID NO. 3)
Phospho-ACGATAAGGGTAGCGGCTCCAAAAAAAAAAAA.

The following components are mixed:

| | |
|---|---|
| RNA from previous step | 200 pmol |
| PolyA-puromycin oligo | 240 pmol |
| Splint oligo | 220 pmol |
| Nuclease-free water | Up to 25.5 μL |

The tube is incubated at 65° C. for 2 minutes, and then incubated on ice for 30 seconds followed by room temperature for 1 minute.

The following components are added directly to the tube:

| | |
|---|---|
| 10 x T4 Ligation Buffer | 3 μL |
| T4 DNA Ligase (400 units/μl) | 1.5 μL |

The tube is then incubated at room temperature for 2 hours, followed by RNA purification using an RNA clean-up kit.

The ligated RNA is digested by Lambda exonuclease to remove the splint.

An exemplary reaction to remove the splint is carried out as follows:

Eluted RNA (from previous step)

| | | |
|---|---|---|
| 10x Lambda exonuclease buffer | 5 | μL |
| Lambda exonuclease | 1.5 | μL |

| | |
|---|---|
| Nuclease-free water | Up to 50 μL |

The reaction is incubated at 37° C. for 1 hour.

### (c) Purification of Ligated RNA

The methods optionally include one or more steps to purify the ligated RNA. An exemplary purification is carried out by oligo-dT beads; RNA purification using oligo-dT beads following manufacturer's protocol, such as DYNABEADS™ mRNA DIRECT™ Purification Kit (61011, Thermo).

### (d) In Vitro Translation

The methods include in vitro translation and use the resulting mRNA to generate the corresponding peptide/protein-mRNA fusion. Post-translation, high concentrations of KCl and MgCl2 are added and incubated at room temperature for 30 minutes to facilitate the fusion between the peptide/protein and RNA.

An exemplary reaction for in vitro translation is carried out as follows:

| Poly-dA RNA from previous step | 250 ng (Max 6 μL) | |
|---|---|---|
| Rabbit Reticulocyte Lysate, Nuclease-Treated | 17.5 | μL |
| Amino acid (-methionine) | 0.5 | μL |
| Amino acid (-leucine) | 0.5 | μL |
| RNAse inhibitor | 0.5 | μL |

| | |
|---|---|
| Nuclease-free water | Up to 25 μL |

The reaction is incubated at 30° C. for 1.5 hours.

For Peptide/protein-mRNA fusion formation, the following components are added directly to every 25 μL in vitro translation reaction.

| | |
|---|---|
| 1M MgCl₂ | 2 μL |
| 3M KCl | 6 μL |

The reaction is incubated at room temperature for 30 minutes or −20° C. overnight.

### (e) Purification of Peptide/Protein-mRNA Fusion

The methods optionally include one or more steps to purify the peptide/protein-mRNA fusion. For example, in some forms, the peptide/protein-mRNA fusion is affinity-purified by the peptide tag using specific antibodies/binders and eluted in specific buffers. For example, commercially available Strep-tactin beads can be utilized to isolate StrepTag-II containing proteins, eluted in commercially available BXT elution buffer.

The reaction of previous fusion formation (33 μL) is be diluted by Strep Wash buffer (100 mM Tris-HCl pH8.0, 150 mM NaCl, 1 mM EDTA, 0.5% TritonX-100) to 100 uL. Then, 5 μL of MagStrep Type 3 XT Beads (IBA Lifesciences) is added and incubated at room temperature for 3 hours or 4° C. overnight. The beads can then be retained by magnet and washed by Strep Wash buffer for 3 times and Strep Wash buffer without Triton X-100 once. The peptide/protein-mRNA fusion bound by the beads is eluted in 5 μL BXT buffer (IBA Lifesciences) at room temperature for 30 minutes.

iii. cDNA Synthesis to Generate Peptide/Protein-mRNA-cDNA Fusion Complex

The methods prepare peptide/protein-mRNA-cDNA fusion complex (“Fusion Complex” in short in following steps). In an exemplary form, the peptide/protein-mRNA fusion prepared according to the methods is subject to reverse transcription to synthesize cDNA, to provide a peptide/protein-mRNA-cDNA fusion complex. The following protocol (step f) illustrates how the described methods for cDNA synthesis to generate peptide/protein-mRNA-cDNA fusion complex can be incorporated into the workflow for the identification of antibody epitopes by mRNA display.

### (f) Reverse Transcription

The methods include in vitro reverse transcription of the peptide/protein-mRNA fusion. An exemplary reverse transcription reaction is carried out as follows:

5 μL Purified Peptide/protein-mRNA fusion eluted from previous step (e) (in 1×BXT buffer) is mixed with:

| | | |
|---|---|---|
| MgCl₂ | 2 | μL |
| 0.1M DTT | 1 | μL |
| RNase OUT | 0.25 | μL |
| SuperScript III RT enzyme | 0.125 | μL |
| (200 U/μL, #18080-044, Life Technologies) | | |
| 10 mM dNTP | 0.5 | μL |
| Display-amp-R (10 uM) | 2.5 | μL |

| | |
|---|---|
| Nuclease-free water | (Up to 10 μL) |

The reaction should be incubated at 42° C. for 1 hour. After the reaction, a small proportion should be saved as input for NGS analysis.

2. Immuno-Capture of mRNA-Display Epitope Library

In some forms, methods for immuno-capture of mRNA-display epitope library include multiple steps, including (iv) Capture of Antibody; (v) Immuno-capture of fusion complex; (vi) Elution of fusion complex; (vii) PCR amplification and barcoding; and (viii) Preparation of sequencing library.

iv. Capture of Antibody

The methods include steps for capturing antibody. The following protocol (steps g-j) illustrates how the described methods for antibody capture can be incorporated into the workflow for the identification of antibody epitopes by mRNA display. In some forms, the methods include (g) coating, (h) blocking, (i) antibody capture, and (j) washing.

### (g) Coating

The methods include one or more steps of coating a solid matrix with antibodies. Exemplary solid matrices include wells or beads. In an exemplary form, protein binders that can be used to purify specific class or subclass of antibodies are coated or bound on the surface of multi-well plates (such as 96-well plate) or beads.

### (h) Blocking

The methods include one or more steps of blocking to prevent non-specific binding. In an exemplary form, blocking buffer containing a mixture of detergent, proteins, RNA and DNA (such as 0.1% Tween-20, 5% bovine serum albumin, 1% fish gelatin, 40 pg/ml yeast tRNA and 40 pg/ml salmon sperm DNA in phosphate-buffered saline (PBS)) is used to cover all the inner surface of the wells or beads to prevent non-specific binding during the following steps. The RNA and DNA should be not overlapping with the DNA sequences of epitopes, in order to avoid contaminating the final sequencing data.

### (i) Antibody Capture

The methods include one or more steps of antibody capture. In an exemplary form, body fluid of infected animals or human subjects containing antibodies are added into the wells and incubated at room temperature or 4° C. for 4 hours or overnight, so that specific (sub-)types of antibodies can be captured by the protein binders.

### (j) Washing

The methods include one or more steps of washing away unbound antibody and reagents. In an exemplary form, after capturing, the plates are washed extensively with wash buffer in order to remove non-captured proteins, particularly the proteases and nucleases in body fluid. An exemplary wash buffer contains high concentration of detergent (such as 0.5% Triton-X100 and 0.1% Tween in phosphate-buffered saline).

v. Immuno-Capture of Fusion Complex

The methods include one or more steps of immuno-capture of fusion complex. In an exemplary form, the solution containing a pool of fusion complexes (from Part 1) is diluted in blocking buffer and added to the wells or beads with antibody captured from previous step, so that peptides/proteins bound by the antibodies can be immunoprecipitated. 2 hours after incubation at room temperature (such as 22° C.), the solution should be aspirated, and the wells should be washed with wash buffer for 4 times.

vi. Elution of Fusion Complex

The methods include one or more steps of Elution of fusion complex. In an exemplary form, the immuno-captured fusion complexes are eluted by one or more of the following methods:
Cleaving F(ab)2′ of specific types of antibodies using enzymes (such as IdeZ);
Reducing agent (such as DTT);
Acidic/alkaline solutions;
Heat.
   vii. PCR Amplification and Barcoding

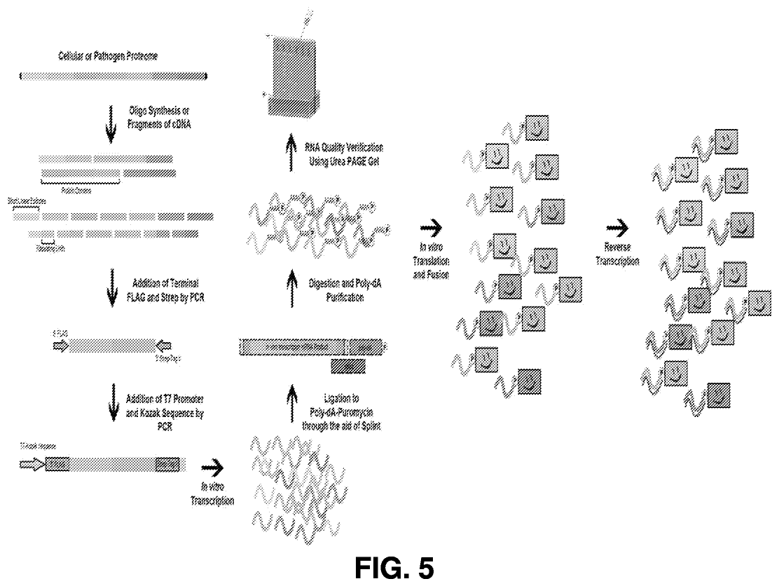

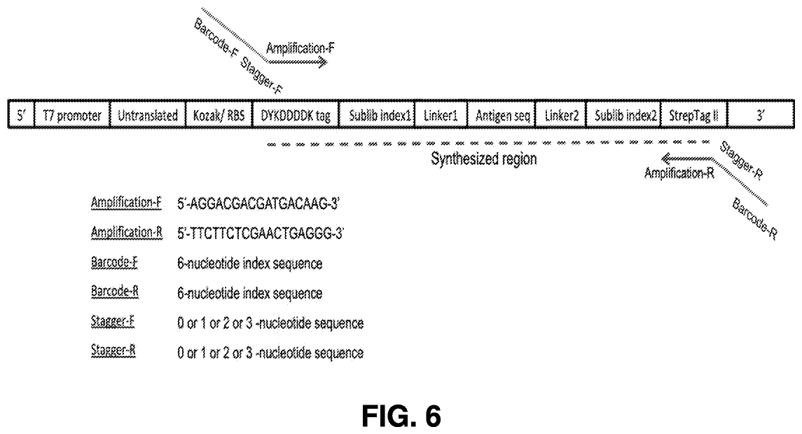

The methods include one or more steps of PCR amplification and barcoding. In an exemplary form, both the input and the eluted fusion complexes are amplified by PCR. The primers can anneal to the constant regions at both 5′ and 3′ ends (i.e., sequences encoding tags) on the input library, while containing flanking barcodes that are used to distinguish each sample (FIG. 6).

viii. Preparation of Sequencing Library

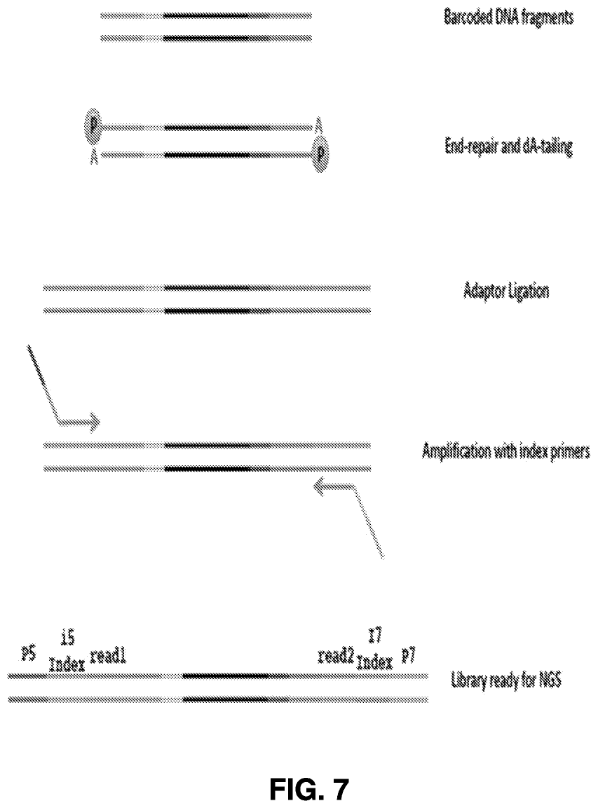

The methods include one or more steps of Preparation of a sequencing library. In an exemplary form, barcoded PCR product is pooled and used to prepare sequencing library. Exemplary methods for preparing a sequencing library include one or more of end-repair, dA-tailing, adaptor ligation and PCR amplification (FIG. 7).

C. Using mRNA-Display and Nano-Droplet Sequencing to Determine Epitope-Specific B Cell Receptor Sequences En Masse

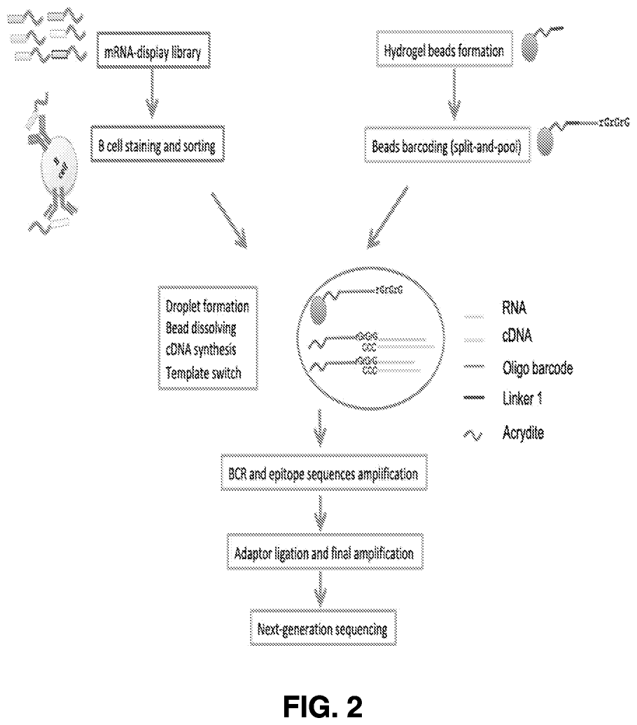

Methods for the isolation and characterization of epitope-specific B cells are provided. The barcoded epitopes obtained according to the methods for identification of antibody epitopes by mRNA display, set forth above, are labelled with fluorophore, to stain and isolate B cells with receptors (B cell receptor, BCR) recognizing these epitopes. The cells isolated are then analyzed by single-cell nano-droplet sequencing to establish the pairing the B cell receptors and epitopes on the cell surface (FIG. 2). The methods offer the option to profile the B cell transcriptome at single-cell level if needed. Methods for the isolation and characterization of epitope-specific B cells include three steps, including (1) Preparation of mRNA-display library and cells, (2) Preparation of bar-coded beads, and (3) Encapsulation of single-cell and single-bead into droplet and sequencing library preparation. Exemplary protocols for performing the methods are set forth below.

1. Preparation of mRNA-Display Library and Cells

Methods for preparation of mRNA display libraries and cells include steps for Preparation of a DNA library and for Preparation of peptide/protein-mRNA fusion complex as set forth above.

i. Preparation of DNA Library

The methods include preparation of an mRNA-display library, as set forth above. Typically, a nucleic acid display library should include wild-type or mutant epitopes (peptides/domains) derived from a particular species, such as pathogens (e.g., viruses, bacteria, fungi etc.), mammalian cells (e.g., for autoantigens or tumor antigens), or other species (e.g., allergens). Typically, DNA Library preparation includes pooling multiple nucleic acids encoding the target epitopes. The nucleic acid encoding the target epitopes can be synthesized as a pool by one or more methods. Exemplary methods include on-chip DNA synthesis technologies, synthesis of regular oligo containing mutant cassettes, and fragmented from genomic or cDNAs. In some forms the pool contains as many as hundreds, thousands, tens of thousands, hundreds of thousands, millions, or tens of millions of DNA fragments. Typically, the upper limit of nucleic acid fragments is determined by oligo synthesis capacity, or by the availability of genomic/cDNA libraries. In some forms, the methods include one or more systems or methods for the design of the oligo pool. For example, in some forms, synthetic DNA fragments are linked with a promoter sequence (e.g., T7 promoter sequence), a nucleic acid motif that functions as the protein translation initiation site (e.g., Kozak consensus sequence or Kozak sequence), and sequence encoding a first peptide tag (e.g., DYKDDDDK (SEQ ID NO. 1) tag) at the 5′ end; and sequence encoding a second peptide tag (e.g., Strep-tagII) at their 3′ end, by Polymerase Chain Reaction (PCR).

ii. Preparation of Peptide/Protein-mRNA Fusion Complex

The methods include steps for preparation of a peptide/protein-mRNA fusion complex, as set forth above. In some forms, methods for identifying antibody epitopes by mRNA display include a step of preparation of peptide/protein-mRNA fusion complex. An exemplary scheme for preparation of peptide/protein-mRNA fusion complex is illustrated in FIG. 5. The protocols (steps a-e) set forth above illustrate how the described methods for preparation of peptide/protein-mRNA fusion complex can be incorporated into the workflow for identification of antibody epitopes by mRNA display.

Preparation of mRNA-Display Library and Cells
iii. Generate Peptide/Protein-mRNA-cDNA Fusion Complex with Fluorescent Labeling (Fluorescent Fusion Complexes)

The methods include steps for reverse transcription on the peptide/protein-mRNA fusion complex. In an exemplary form, Reverse transcription on the peptide/protein-mRNA is performed using biotin-modified primer, which anneal to both oligo dA and constant region of the mRNA. In an exemplary form, the resulting peptide/protein-mRNA-cDNA fusion complex is purified and immobilized by an antibody or binding protein against the tag on the peptide/protein (such as anti-DYK antibody against DYKDDDDK (SEQ ID NO. 1) tag).

In some forms, fluorophore-conjugated streptavidin is added to bind the biotin on the cDNA after washing off the free oligos.

After washing off the free streptavidin, specific fusion complexes are eluted from antibodies by competitive peptides, such as using purified DYKDDDDY (SEQ ID NO. 1) peptide to elute fusion complex from anti-DYK antibody. By this means, the fusion complexes are labeled with fluorophore and ready to be used for staining B cells.

iv. Cell Preparation

The methods include steps for isolating Single B cells. In an exemplary form, B cells are isolated from human or animals can be from fresh or properly frozen blood, purified lymphocytes or tissues using selection kits, or staining by antibodies recognizing B cell marker(s).

v. B Cell Staining and Sorting

The methods include steps for staining and sorting of B cells. In an exemplary form, B cells are stained by both fusion complex (for identification of epitopes) and B cell marker (for identification of B cells), before subject to flow cytometry sorting.

Typically, cells are first incubated with fluorescent fusion complexes in staining buffer, such as PBS containing 2% Fetal Bovine Serum on ice for 30 minutes. After staining, cells are washed by staining buffer 3 times.

In some forms, cells are incubated with staining buffer containing RNaseH and a fluorophore-labeled antibody cocktail, which includes anti-cell surface marker antibodies for other blood cells and (subtypes of) B cells. The cells are incubated on ice for 30 minutes, and then washed with staining buffer for 3 times. Cells are kept on ice in staining buffer before flow cytometry sorting.

The methods include steps for flowcytometry sorting of cells. Typically, each subtype of B cells with streptavidin staining positive is collected.

### 2. Preparation of Barcoded Beads

The methods include steps for preparation barcoded beads. There are multiple ways to prepare barcoded epitopes in parallel. One exemplary form is presented below.

vi. Hydrogel Beads Formation

The hydrogel beads should be formed as previous reports (Klein, A. M. et al., Cell 161, 1187-1201, (2015)). Briefly, a microfluidic device is equipped with a flow-focusing junction at which the continuous stream of aqueous phase (continuous phase) is emulsified into a stream of highly mono-disperse droplets (disperse phase). The droplets should be collected off-chip and polymerized into Hydrogel beads.

The composition of the dispersed phase is 10 mM Tris-HCl [pH 7.6], 1 mM EDTA, 15 mM NaCl containing 6.2% (v/v) acrylamide, 0.18% (v/v) bis- acrylamide, 0.3% (w/v) ammonium persulfate and 50 μM acrydite-modified DNA primer. For continuous phase, fluorinated fluid HFE-7500 carrying 0.4% (v/v) TEMED and 2.0% (w/w) EA-surfactant were used.

Droplets should be collected into a 1.5 mL tube and incubated at 65° C. for 12 hours to allow polymerization. The resulted solidified beads are released into bulk and washed twice with 1 mL of 20% (v/v) 1H,1H,2H,2H-perfluorooctanol (B20156, Alfa Aesar) TEBST buffer (10 mM Tris-HCl [pH 8.0], 137 mM NaCl, 2.7 mM KCl, 10 mM EDTA and 0.1% (v/v) Triton X-100). The beads will carry an acrydite (Ac)-modified DNA primer, which includes 5′-Ac-Photo-cleavable spacer (PC)-Linker1-3′ for subsequent barcoding.

vii. Split-Pool Combinatorial Barcoding of Hydrogel Beads

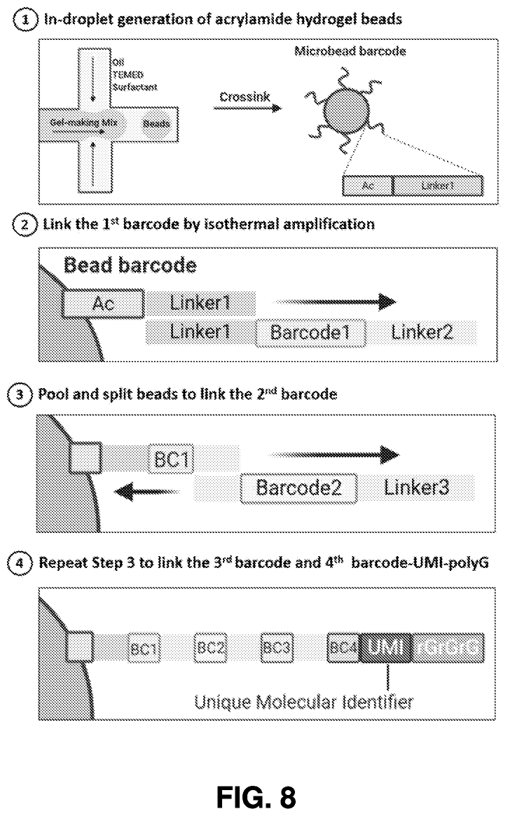

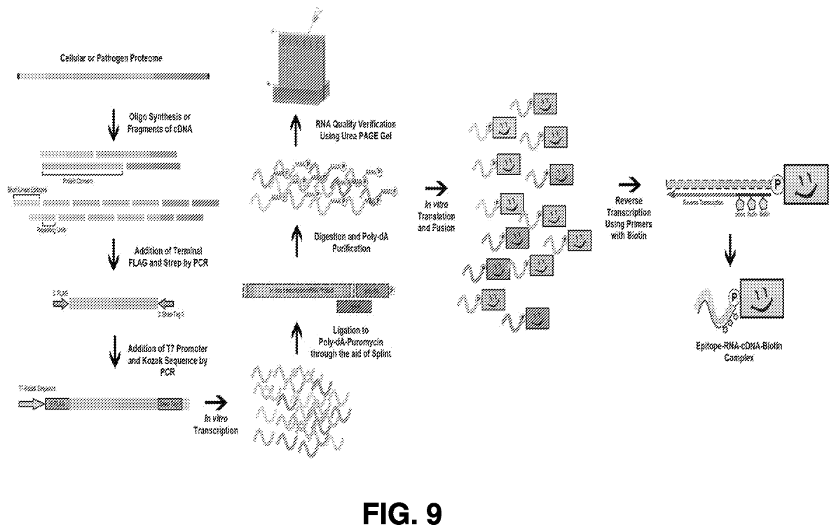

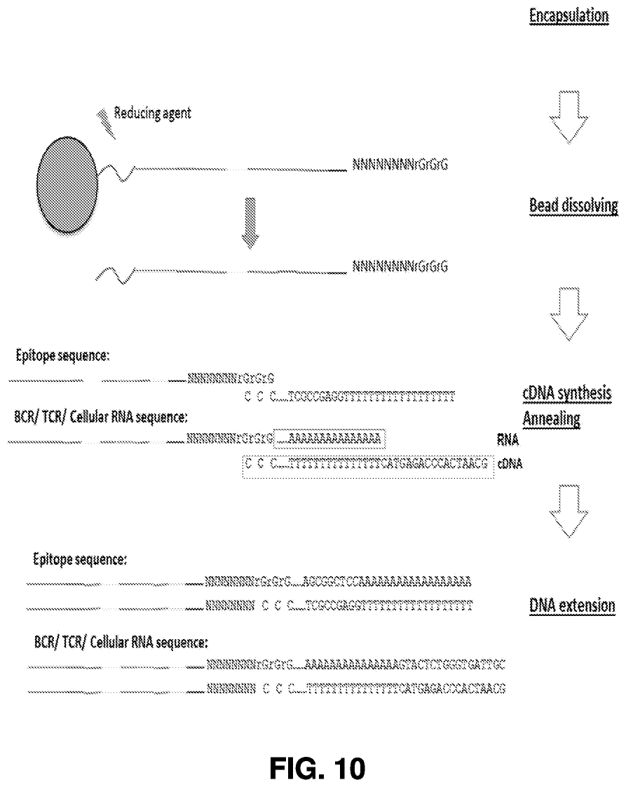

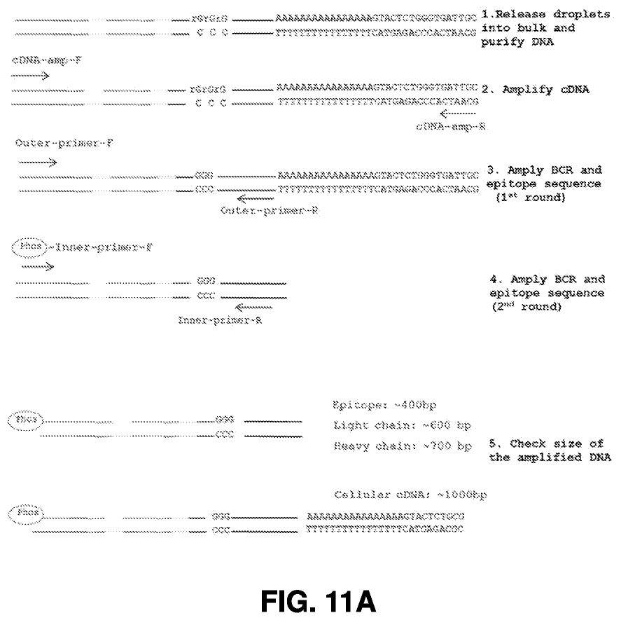

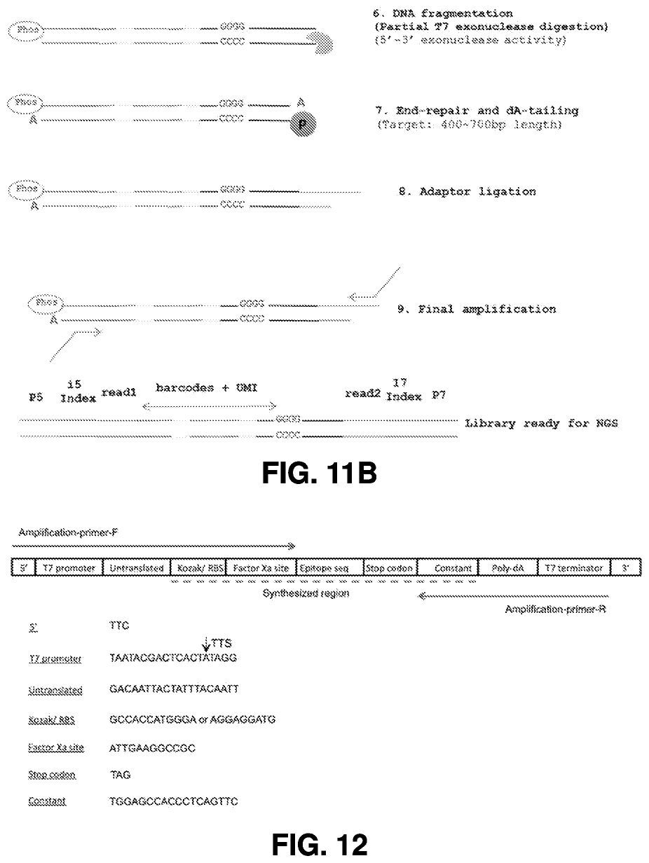

To prepare barcoded primers on the hydrogel microspheres, a four-step enzymatic extension reaction were used, similar with previous publication (FIG. 8) (Klein, A. M. et al., Cell 161, 1187-1201, (2015)). To begin, a 96-well 2 mL plate was pre-loaded with primer encoding the first barcode region (5′-Linker1-BC1-Linker2-3′, where ‘BC1′ indicates the first barcode region for each well).

To link the first barcode by isothermal amplification, 150 uL is added to the reaction mix, containing ˜8,847,360 hydrogel beads (carrying 5′-Ac-PC-Linker1-3′ primer), isothermal amplification buffer (NEB) and dNTP into each well. DNA is denatured at 85° C. for 2 minutes, followed by hybridization between bead tagged-primer and BC1-encoded primer at 60° C. for 20 minutes. After that, Bst polymerse is added to the mixture, so that the final volume of each well is 200 μL.
Bst 2.0: 1.8 U
dNTP: 0.3 mM
1× isothermal amplification buffer: Up to 50 μL

The mixture is incubated at 60° C. for 1 hour, followed by stopping in 200 μL of stop buffer (100 mM KCl, 10 mM Tris-HCl [pH 8.0], 50 mM EDTA, 0.1% (v/v) Tween-20) on ice for 30 min. The beads will now contain 5′-Ac-PC-Linker1-BC1-Linker2-3′ primer.

After collecting and pooling the beads together, they are subject to the following washes:
A) Stop buffer containing 10 mM EDTA for 3 times,
B) Alkaline buffer (150 mM NaOH, 0.5% (v/v) Brij 35) for 3 times to remove the second strand, and
C) TET buffer (10 mM Tris- HCl [pH 8.0], 10 mM EDTA, 0.1% (v/v) Tween-20) for 3 times.

Finally, beads were suspended in 13 mL of TET buffer.

In some forms, the methods repeat the above procedure for the second, third and fourth barcoding steps as outlined below:
A) The second barcoding primer: 5′-Linker2-BC2-Linker3-3′ (where ‘BC2′ indicates a unique sequence for each well). After amplification, the beads will contain 5′-Ac-PC-Linker1-BC1-Linker2-BC2-Linker3-3′ primer;
B) The third barcoding primers: 5′-Linker3-BC3-Linker4-3′ (where ‘BC3′ indicates a unique sequence for each well). After amplification, the beads will contain 5′-Ac-PC-Linker1-BC1-Linker2-BC2-Linker3-BC3-Linker4-3′ primer;
C) The fourth barcoding primer: 5′-Linker4-BC4-UMI-rGrGrG-3′ (where ‘BC4′ indicates a unique sequence for each well and “UMI” (unique molecular identifier) is a random octa-nucleotide). After amplification, the beads will contain 5′-Ac-PC-Linker1-BC1-Linker2-BC2-Linker3-BC3-Linker4-BC4-UMI-rGrGrG-3′ primer.
   3. Encapsulation of Single-Cell and Single-Bead into Droplet and Sequencing Library Preparation

The methods include steps for encapsulation of single-cell and single-bead into droplet and sequencing library preparation. One exemplary form for encapsulation of single-cell and single-bead into droplet and sequencing library preparation is presented below.

viii. Encapsulation and cDNA Synthesis

The methods include steps for Encapsulation and cDNA synthesis. In an exemplary form the cell encapsulation process follows the procedure of a published study (Klein, A. M. et al., Cell 161, 1187-1201, (2015)), which relies on random arrival of cells into the device. After encapsulation, the sequencing library preparation procedure can follow the commonly used template switch oligo (TSO) or polyA capturing, with corresponding modifications on the fourth barcoding primers. One exemplary method of the TSO process is set forth below.

To minimize two or more cells from entering the same droplet, the cells are encapsulated with an average occupancy of 1 cell in 5-10 droplets, by diluting cell suspensions to ˜50-100,000 cells/mL.

To prevent cell sedimentation in the syringe or other parts of the system, cells are suspended in 1×PBS buffer with 16% (v/v) density gradient solution OPTIPREP (Sigma). Typically, the methods use 20,000 cells suspended in 160 μL 0.5×PBS (17-516F, Lonza), 32 μL Optiprep (1114542, Axis-Shield) and 8 μL 1% (v/v) BSA (B14, Thermo Scientific), in a total volume 200 μL. Cells remain in suspension using a micro-stir bar placed in the syringe and rotated using a magnet attached to a rotating motor.

In an exemplary form, the production of droplets containing barcode and the reverse transcription components is carried out using the following reverse transcription/lysis mix:
5× First-Strand buffer (18080-044 Life Technologies) 25 μL
25 mM dNTPs: 6 μL
0.1M DTT: 10 μL
1M Tris- HCl, pH 8.0: 15 μL
Murine RNase inhibitor (M0314, NEB): 10 μL
SUPERSCRIPT III RT enzyme (200 U/pL, #18080-044, Life Technologies): 15 μL

10uM RT primer
(SEQ ID NO. 4):
(TTTTTTTTTTTTTTTTCATGAGACCCACTAACG) 15 μL

Nuclease-free water: Up to 150 μL

HFE-7500 fluorinated fluid (3M) will then be used as carrier oil, with 2.0% (w/w) EA surfactant (RAN Biotechnologies) to provide equilibrium interfacial tension. After cell encapsulation, the beads will be dissolved due to the presence of DTT. For reverse transcription, the tube is incubated at 42° C. for 1 hour to allow cDNA synthesis and template switch.

ix. Demulsification and DNA Purification

The methods include steps for Demulsification and DNA purification. In an exemplary form the emulsion is demulsified by adding 1 volume of PFO solution (20% (v/v) perfluorooctanol and 80% (v/v) HFE-7500). The aqueous phase from the broken droplets should be transferred into a new tube and processed to sequencing library preparation as described below.

x. cDNA Amplification

The methods include steps for cDNA amplification. In an exemplary form the Barcoded DNA in the aqueous phase is purified by 2.0× volume of magnetic beads (e.g., Ampure XP beads, Beckman Coulter) and eluted in 20 μL of nuclease-free water.

The cDNA should then be amplified by PCR for 10-15 cycles using forward primer annealing to the 5′ Linker1 and reverse primer annealing to the constant region of cDNA at 3′. The resulted dsDNA should undergo size selection to separate the epitope sequence (−400 bp) and cellular mRNA transcriptome (mostly >500 bp) using magnetic beads.

cDNA-Amp-F:
(SEQ ID NO. 5)
ACGACGCTCTTCCGATCT
cDNA-Amp-R:
(SEQ ID NO. 6)
GGAGCCGCTACCCTTATC

xi. Epitope and BCR Sequence Amplification

The methods include steps for Epitope and BCR sequence amplification. In an exemplary form, the epitope sequence and BCR sequence are amplified by PCR from the dsDNA from previous step.
1) PCR reaction for epitope sequence amplification:
First-step amplification
dsDNA (−400 bp): 10 μL
2×Q5 master mix: 12.5 μL
Linker1-Outer-F (10 μM): 1.25 μL
Epitope-Outer-R (10 μM): 1.25 μL
Total: 25 μL
Exemplary Thermocycling conditions are as follows:

| Temperature (° C.) | Time (second) |
|---|---|
| Step 1 98 | 30 |
| Step 2 98 | 5 |
| Step 3 65 | 10 |
| Step 4 72 | 20 |
| Step 5 (Go to Steps 2-4, repeat for 7 cycles); | |
| Step 6 72 | 120 |
| Step 7 4 | hold |

The product should be purified by 2.0× volume (50 μL) of magnetic beads and eluted in 12 μL of nuclease-free water.

Second-step Amplification is carried out using the following reagents:
dsDNA (˜400 bp): 10 μL
2×Q5 master mix: 12.5 μL
Linker1-Inner-F (10 μM): 1.25 μL
Epitope-Inner-R (10 μM): 1.25 μL
Total: 25 μL

Exemplary Thermocycling conditions are as follows:

| Temperature (° C.) | Time (second) |
|---|---|
| Step 1 98 | 30 |
| Step 2 98 | 5 |
| Step 3 65 | 10 |
| Step 4 72 | 20 |
| Step 5 (Go to Steps 2-4, repeat for 7 cycles); | |
| Step 6 72 | 120 |
| Step 7 4 | hold |

The product should be purified by 2.0× volume (50 μL) of magnetic beads and eluted in 12 (° C.) of nuclease-free water.

### 2) PCR Reaction for BCR Sequence Amplification:

First-step amplification:
dsDNA (˜400 bp): 10 μL
2×Q5 master mix: 12.5 μL
LinkerA-Outer-F (10 μM): 1.25 μL
BCR-Outermix-R (10 μM): 1.25 μL
Total: 25 μL

Exemplary Thermocycling conditions are as follows:

| Temperature (° C.) | Time (second) |
|---|---|
| Step 1 98 | 30 |
| Step 2 98 | 5 |
| Step 3 65 | 10 |
| Step 4 72 | 20 |
| Step 5 (Go to Step 2-4, repeat for 7 cycles); | |
| Step 6 72 | 120 |
| Step 7 4 | hold |

The product should be purified by 1.0× volume (25 μL) of magnetic beads and eluted in 12 μL of nuclease-free water.
Second-step amplification:
dsDNA (˜400 bp): 10 μL
2×Q5 master mix: 12.5 μL
LinkerA-Inner-F (10 μM): 1.25 μL
BCR-Innermix-R (10 μM): 1.25 μL
Total: 25 μL

Exemplary Thermocycling conditions are as follows:

| Temperature (° C.) | Time (second) |
|---|---|
| Step 1 98 | 30 |
| Step 2 98 | 5 |
| Step 3 65 | 10 |
| Step 4 72 | 20 |
| Step 5 (Go to Step 2-4, repeat for 7 cycles); | |
| Step 6 72 | 120 |
| Step 7 4 | hold |

The product should be purified by 1.0× volume (25 μL) of magnetic beads and eluted in 12 μL of nuclease-free water.

xii. Fragmentation

The methods include steps for Fragmentation. In an exemplary form, the eluted dsDNA of epitope and BCR sequences is subject to fragmentation in separate reactions, both using T7 exonuclease but with different reaction time.

### 1) For Epitope Sequences:

dsDNA (˜380 bp): 50 ng
NEBuffer 4 (10×): 2.5 μL
T7 Exonuclease: 0.5 μL
Nuclease-free water: up to 25 μL

Incubate at 25° C. for 15 minutes and stop reaction by adding EDTA to at least 11 mM. The product should be purified by 2.0× volume (50 μL) of magnetic beads.

### 2) For BCR Sequence:

dsDNA (˜700 bp): 200 ng
NEBuffer 4 (10×): 2.5 μL
T7 Exonuclease: 0.5 μL
Nuclease-free water: up to 25 μL

Incubate at 25° C. for 30 minutes and stop reaction by adding EDTA to at least 11 mM. The product should be purified by 1.0× volume (25 μl) of magnetic beads.

xiii. End-Repair/dA-Tailing and Adaptor Ligation.

The methods include steps for End-repair/dA-tailing and adaptor ligation. In an exemplary form, the End-repair and dA tailing is performed by commercially available enzyme mixture following manufacture's protocol, such as NEBNEXT® Ultra™ II End Repair/dA-Tailing Module (E7546S), in order to add a phosphate group at 5′ prime at the downstream of dsDNA and a dA at the 3′ prime of both sides. Afterwards, DNA adaptor encoding the read2 primer of Illumina sequencing or other sequencing platforms should be added by DNA ligase to the downstream end of dsDNA. Commercially available enzyme can be used, such as NEBNEXT® ULTRA™ II Ligation Module (E7546S).

An example of End-repair/dA tailing and adaptor ligation protocol is described below:

### 1) End Repair/dA Tailing

DNA (from previous step): 50 ng
End repair Buffer: 1.75 μL
End repair enzyme mix: 0.75 μL
Nuclease-free water: Up to 15 μL
Exemplary Thermocycling conditions are as follows: (lid temperature >75° C.)

| Temperature (° C.) | Time (minutes) |
|---|---|
| Step1 20 | 30 |
| Step2 65 | 30 |

### 2) Ligation

Add the following components directly to the tube.
End repair reaction (from previous step): 15 μL
Ligation Master mix: 7.5 μL
Ligation Enhancer: 0.25 μL
Adaptor (25 μM): 1 μL
Exemplary Thermocycling conditions are as follows: (lid off)

| Temperature (° C.) | Time (minutes) |
|---|---|
| Step1 20 | 15 |

The resulted fragment should be purified by 1.5× volume of magnetic beads (35.625 μL) and eluted in 12 μL of nuclease-free water.

xiv. PCR Amplification of the Sequencing Library

The methods include steps for PCR amplification of the sequencing library. In an exemplary form, the ligated DNA fragments are amplified by PCR with primers annealing to the Linker1 at the upstream end and Adaptor at the downstream end. P5, i5 index, P7 and i7 index (for Illumina NGS) will be introduced by the primers.
DNA fragment (purified from ligation reaction) 10 μL
2×Q5 master mix: 12.5 μL
i5 primer (25 μM): 1.25 μL
i7 primer (25 μM): 1.25 μL
Total: 25 μL
Exemplary Thermocycling conditions are as follows:

| Temperature (° C.) | Time (second) |
|---|---|
| Step 1 98 | 30 |
| Step 2 98 | 5 |
| Step 3 65 | 10 |
| Step 4 72 | 20 |
| Step 5 (Go to Step 2-4, repeat for repeat for 5 cycles); | |
| Step 6 72 | 120 |
| Step 7 4 | hold |

In some forms the methods determine the BCR sequences of over 1 million B cells enquired by over 1 million epitopes. The methods provide a comprehensive description of the B cell responses in an individual, which will enable an accurate assessment of the immune status, important for proper therapy and prevention of diseases.

D. Using mRNA-Display and Nano-Droplet Sequencing to Determine Epitope-Specific T Cell Receptor Sequences En Masse

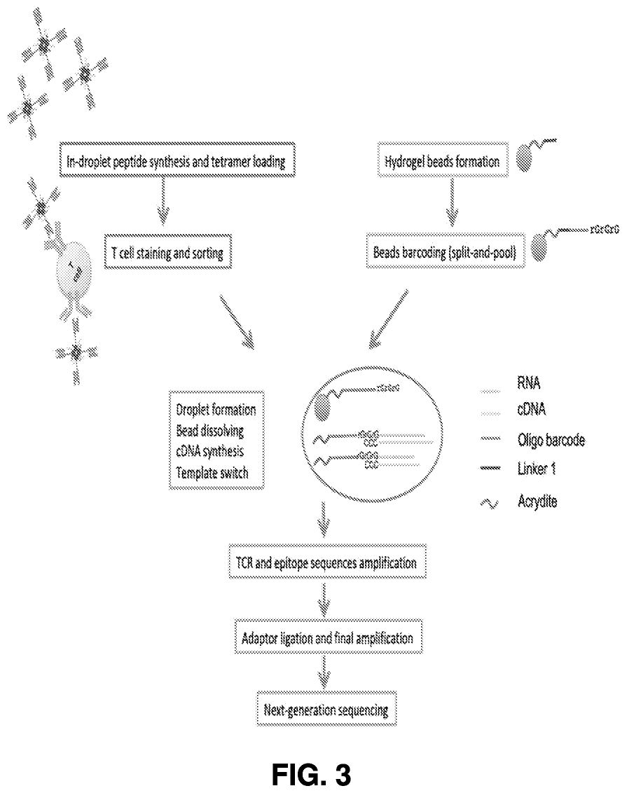

Methods for the isolation and characterization of epitope-specific T cells are provided. T cell receptors can only recognize epitopes that are presented by major histocompatibility complexes (MHCs). Therefore, each epitope (synthesized from one particular DNA fragment by in vitro transcription and translation within a droplet) will be loaded into fluorophore-labeled MHC oligomers, while the intermediate mRNA product can be used to barcode the peptide-specific tetramer. These MHC-oligomers can be utilized to stain and isolate peptide-specific T cells. The oligomer-linked barcode, T cell receptor sequence and transcriptome can be analyzed at single-cell level by droplet sequencing (FIG. 3). The methods offer the option to profile the T cell transcriptome at single-cell level if needed.

Methods for the isolation and characterization of epitope-specific T cells include four steps, including (1) Preparation of barcoded tetramers in droplets, (2) T Cell staining and sorting, (3) Preparation of barcoded beads and (4) Encapsulation of single-cell and single-bead into droplet and sequencing library preparation. Exemplary protocols for performing the methods are set forth below.

### 1. Preparation of Barcoded Tetramers in Droplets

Methods for preparation of barcoded tetramers in droplets include steps for Preparation of a DNA library, Preparation of fluorophore and oligo labeled streptavidin (FOS), or fluorophore labeled mono-avidin on branched DNA (FMbD), and for Assembly of barcoded MHC-tetramers/oligomers in droplets.

i. DNA Library Preparation

The methods include steps for DNA Library preparation. In an exemplary form, the mRNA display library contains wild-type or mutant predicted epitopes derived from a particular species, such as pathogens (e.g., viruses, bacteria, fungi, etc.), mammalian cells (e.g., for auto-antigens or tumor antigens), or other species (e.g., allergens). DNA encoding the target epitopes can be synthesized as a pool by: (1) on-chip DNA synthesis technologies or (2) synthesis of regular oligo containing mutant cassettes. In an exemplary form, the DNA Library preparation pool contains as many as millions of DNA fragments with the upper limit determined by oligo synthesis capacity.

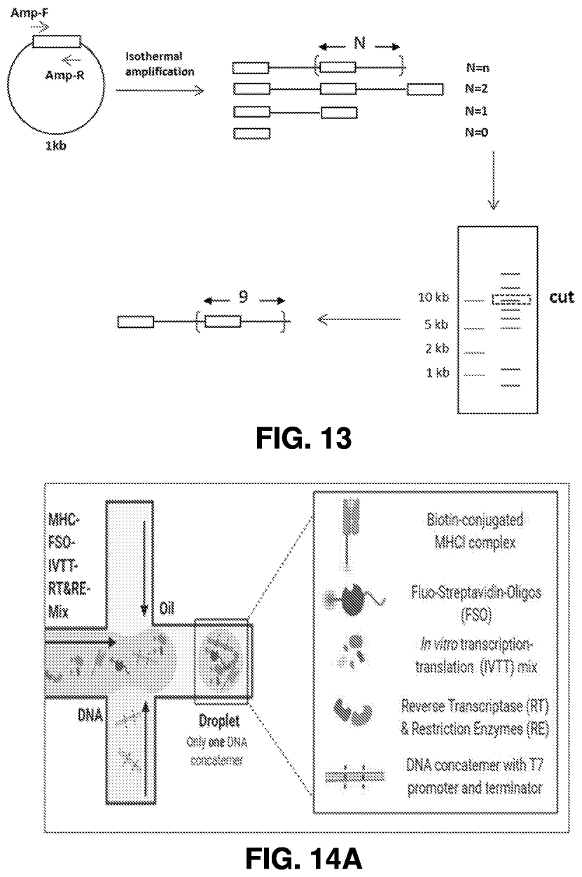

Regarding the design of the oligo pool, synesthetic DNA fragments are linked with a T7 promoter, a ribosome binding site, a start codon, and a factor X recognition site sequence at their 5′ end; and poly-dA and T7 terminator at their 3′ ends by PCR. Because in the downstream steps, DNA concatemers are digested by a restriction enzyme, the enzyme site should be included in the DNA sequence at the 3′ site but not in the epitope encoding region (FIG. 12). The resulting double-stranded DNA is amplified within a physically limited space. In an exemplary form, two approaches are available. In one form, self-circularization and isothermal amplification to form concatemers of each DNA variant (FIG. 13). After purification, the concatemers are encapsulated into droplets for in vitro transcription and translation (IVTT) in the downstream steps. In another form, DNA amplification is used in hydrogel beads. For example, in some forms, the amplification of DNA provides enough templates for in vitro transcription to produce a sufficient amount of epitope peptides to load onto the MHC gloves in droplets in step (iii), below.

ii. Preparation of Fluorophore and Oligo Labeled Streptavidin (FOS), or Fluorophore Labeled Mono-Avidin on Branched DNA (FMbD)

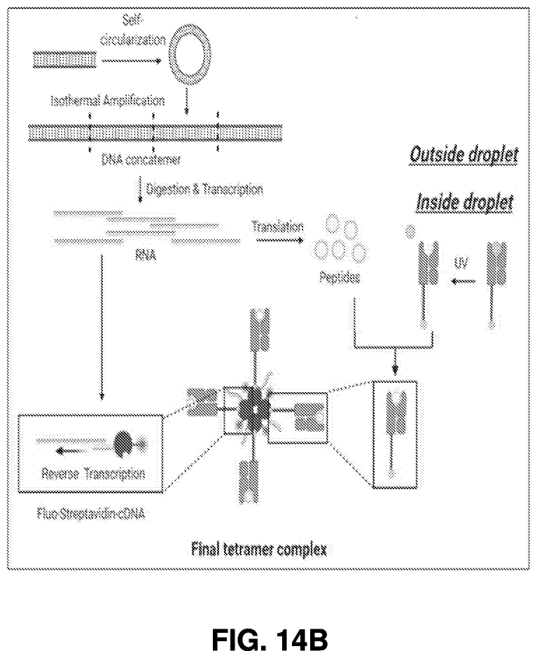

The methods include steps for Preparation of fluorophore and oligo labeled streptavidin (FOS), or fluorophore labeled mono-avidin on branched DNA (FMbD). In an exemplary form, fluorophore- and Oligo-labeled Streptavidin (FSO) is firstly generated by conjugating a DNA oligo to commercially available fluorophore-labeled streptavidin, such as PE-streptavidin and Alexa Fluor 647-streptavidin to form MHC-tetramers. The methods achieve conjugation using a commercial conjugation kit following the manufacturers protocol, such as soloLink Protein-Oligonucleotide Conjugation Kit. Biotinalyated MHC can therefore bind to the streptavidin by strong affinity binding (FIG. 14).

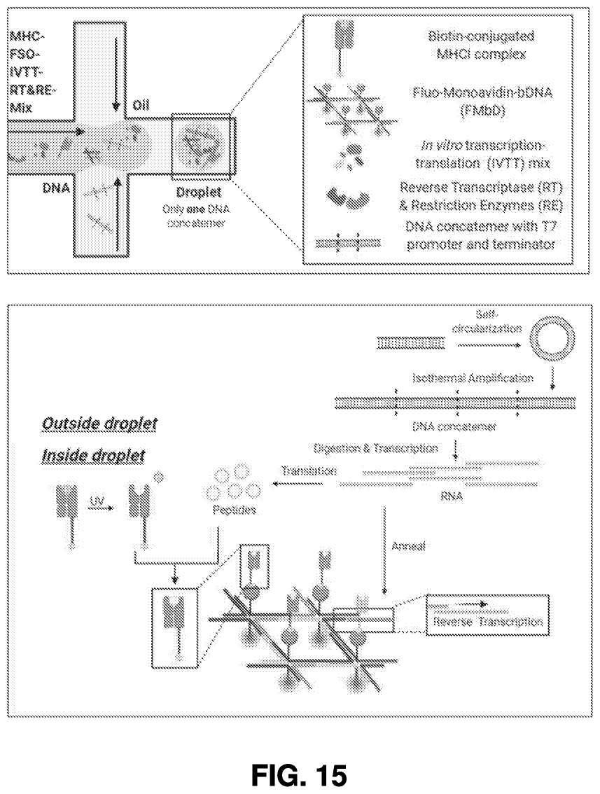

In an alternative form streptavidin is not used, and branched DNA (bDNA) is used as a scaffold to form MHC-oligomers. Monomer avidin and fluorophore is conjugated to bDNA by chemical reaction, followed by biotinalyated MHC binding to the avidin by strong affinity binding (FIG. 15).

iii. Assembly of Barcoded MHC-Tetramers/Oligomers in Droplets

The methods include steps for Assembly of barcoded MHC-tetramers/oligomers in droplets. In an exemplary form, the barcoded MHC-tetramers or MHC-oligomers are formed by in-droplet IVTT reaction, DNA-RNA hybridization and cDNA synthesis by reverse transcription (FIG. 14).

The droplet formation relies on random packaging of individual concatemers into each droplet in the microfluidic device. Therefore, in some forms, the methods minimize two or more concatemers from entering the same droplet, by encapsulating the concatemers with an average occupancy of 1 concatemer in 5-10 droplets, by diluting the concatemer to ˜1,000,000 molecules/mL. In some forms, restriction enzymes are added into the IVTT mix to cleave the DNA concatemer, in order to avoid the generation of RNA concatemers.

For the production of droplets containing barcode and the IVTT-RT components, the following IVTT-RT reaction mix is prepared and added to the tube. HFE-7500 fluorinated fluid (3M) can be used as carrier oil, with 2.0% (w/w) EA surfactant (RAN Biotechnologies) to encapsulate the concatemer.
DNA concatemer: 25,000 molecules
Pure Express Solution A: 10 μL
Pure Express Solution B: 7.5 μL
FOS (1 mg/ml): 1 μL
SuperScript III RT enzyme (200 U/L, #18080-044, Life Technologies): 1 μL
Restricted enzyme of choice: 1 μL
Nuclease-free water: Up to 25 μL

Next, the droplets should be incubated at 37° C. for 1 hour for IVTT reaction, followed by 42° C. for 1 hour to allow cDNA synthesis. Then the tube should undergo UV treatment to release the pre-existing peptide from the tetramers. The reaction should be incubated at room temperature for 15 minutes to allow the newly synthesized peptide to load onto the MHCs, followed by demulsification to release the tetramers/oligomers into the aqueous phase. The tetramers/oligomers can be purified from the aqueous phase by HA tag on the streptavidin or monomer avidin.

An alternative approach to generate MHC-oligomers is to use isothermal DNA amplification in hydrogel beads within each droplet, then each bead will be packaged into one droplet for IVTT and loading onto MHC.

### 2. T Cell Staining and Sorting

Methods for T Cell staining and sorting include steps for T cell isolation, and for T Cell staining and sorting.

iv. T Cell Isolation

The methods include steps for T cell isolation. In an exemplary form, the Single T cells from human or animals are isolated from fresh blood, properly frozen PBMCs, purified lymphocytes or tissues. Typically, cells are isolated using commercially available selection kits or staining by anti-T cell marker antibodies.

v. T Cell Staining and Sorting

The methods include steps for T Cell staining and sorting. In an exemplary form, the Cells should be stained by both fluorescent MHCs tetramers/oligomers (for identification of epitopes) and T cell marker (for identification of T cells), before subject to flow cytometry sorting. An exemplary protocol is set forth below.

First, cells are incubated with fluorescent MHCs tetramers/oligomers in staining buffer, such as PBS containing 2% Fetal Bovine Serum on ice for 30 minutes. After staining, cells should be washed by staining buffer for 3 times.

Next, cells are incubated with staining buffer containing a fluorophore-labeled antibody cocktail, which contains anti-cell surface marker antibodies for other blood cells and (subtypes of) T cells. The cells should be incubated on ice for 30 minutes, and then washed with staining buffer for 3 times. Cells should be kept on ice in staining buffer before flow cytometry sorting.

Lastly, during flowcytometry sorting, each subtype of T cells with fluorescent-MHC staining positive should be collected.

### 3. Preparation of Barcoded Beads

The methods include steps for Preparation of barcoded beads. In an exemplary form, the methods include the same steps as set forth in “2. Preparation of barcoded beads”, above, for methods of Using mRNA-display and nano-droplet sequencing to determine epitope-specific B cell receptor sequences en masse”.

4. Encapsulation of Single-Cell and Single-Bead into Droplet and Sequencing Library Preparation

The methods include steps for Encapsulation of single-cell and single-bead into droplet and sequencing library preparation. In an exemplary form, the methods include the same steps as set forth in “4. Encapsulation of single-cell and single-bead into droplet and sequencing library preparation”, above, for methods of “Component 2. Using mRNA-display and nano-droplet sequencing to determine epitope-specific B cell receptor sequences en masse.”

The described in-droplet MHC-tetramer/oligomer formation allows forming millions of different MHC-tetramers/oligomers, each carrying one distinct peptide within several hours. Therefore, the methods allow for the analysis of millions of T cells pairing with hundreds and thousands of epitopes at single-cell level for each experiment.

Due to the technical limitations, most of the current immunology studies only investigate a handful of T-cell epitopes of pathological antigens or host autoantigens, which is only a small proportion of T-cell response. In contrast, the described methods can achieve the scale to analyze T cells against all epitopes during a particular immune response. For example, in the Immune Epitope Database and Analysis Resource (web site iedb.org), there are 2878 mouse T-cell epitopes on influenza viruses. Using the described method, it is possible to readily isolate and analyze all influenza virus specific-T cells from each infected mouse at single-cell level, which will provide a comprehensive and detailed understanding of T cell immunity on influenza viral infection.

### E. Methods of Using Immunological Profiles

The methods provide data in the form of an immunological profile, including data relating to immune processes in an individual, sample, or system. In an exemplary form, the methods provide data in the form of an immunological profile for an immune response within an individual. In some forms, the immune response is to a pathogen, to an allergen, to a self-antigen, or to a vaccine.

In some forms, the methods provide data in the form of an immunological profile of adaptive immune responses and identify the immune status of a subject. The immunological profile provides high resolution information at genomic scale and can assist in disease diagnosis, prevention, and treatment.

In some forms, the methods provide data in the form of an immunological profile of adaptive immune responses to inform the dynamics of immune responses in infectious diseases caused by pathogens, such as viruses, bacteria, and fungi. In some forms, the methods include one or more steps of computing one or more pieces of data from the nucleic acid or protein sequence data within an immunological profile. For example, in some forms, the methods develop the most suitable therapeutic approaches based on the nucleic acid or protein sequence data within an immunological profile.

In some forms, the methods provide data in the form of an immunological profile of adaptive immune responses to inform tumor-specific immune responses, identify potential tumor epitopes, advise target-specific immune therapy, and improve cancer immune therapies.

In some forms, the methods provide data in the form of an immunological profile of adaptive immune responses to inform the antigens and immune responses underlying autoimmune diseases and develop corresponding diagnosis and therapeutic approaches.

In some forms, the methods provide data in the form of an immunological profile of adaptive immune responses to inform transplantation-associated immune responses, enable early determination of the tissue rejections and corresponding self-antigens or differentiate opportunistic infections.

In some forms, the methods provide data in the form of an immunological profile of adaptive immune responses to inform precise guidance for vaccine development and monitoring the efficacy of vaccines in a subject. Methods of making and using enhanced vaccines against an antigen are provided. Typically, the methods employ one or more steps to characterize target B cells, or target T cells, or target antibodies, or combinations thereof within a subject. The methods provide enhanced vaccines with improved specificity, and antigen cross-reactivity, whilst preventing the development or reducing the severity of auto-immunity in the subject. In some forms, the methods identify epitope-specific sequences amongst immune receptors in the subject for a multiplicity of epitopes within the antigen; determine which one or more of the multiplicity of epitopes for the antigen have the highest number of epitope-specific sequences in the subject; and preparing the vaccine including one or more of the epitopes having the highest number of epitope-specific sequences in the subject. In a particular form, the methods identify epitope-specific T cell receptor sequences in the subject for a multiplicity of epitopes within the antigen; determine which one or more of the multiplicity of epitopes for the antigen have the highest number of epitope-specific T cell receptor sequences in the subject; and prepare the vaccine including one or more of the epitopes having the highest number of epitope-specific T cell receptor sequences in the subject. In another form, the methods identify a multiplicity of antibody epitopes within the antigen by mRNA display; and prepare the vaccine including one or more of the antibody epitopes. In other forms, the methods include identifying epitope-specific B cell receptor sequences in the subject for a multiplicity of epitopes within the antigen; determining which one or more of the multiplicity of epitopes for the antigen have the highest number of epitope-specific B cell receptor sequences in the subject; and preparing the vaccine for the subject including one or more of the epitopes determined as having the highest number of epitope-specific B cell receptor sequences in the subject.

In some forms, the methods provide data in the form of an immunological profile of adaptive immune responses to inform potential causes of allergy and target-specific reduction of allergy.

The underlying causes of many diseases are still unknown. In some forms, the methods provide data in the form of an immunological profile of adaptive immune responses to inform correlations of immunological markers with some diseases, which will set up a foundation to reveal the causal relationship, and potential diagnosis and/or therapeutic approaches.

### III. Kits

*Kits are also disclosed. The kits can include, for example, reagents necessary to carry out DNA Library preparation; Preparation of peptide/protein-mRNA fusion complex (including In vitro transcription, RNA-poly-dA-puromycin ligation reaction, in vitro translation, Peptide/protein-mRNA fusion, in vitro transcription, cDNA synthesis to generate peptide/protein-mRNA-cDNA fusion complex, and reverse transcription); Immuno-capture of the mRNA-display epitope library (coating, blocking, immunocapture and washing); Immuno-capture of mRNA-display epitope library (elution, PCR amplification and barcoding); and preparation of a sequencing library (end-repair, dA-tailing, adaptor ligation and PCR amplification). The active agents can be supplied alone (e.g., lyophilized), or in as admixtures/compositions. The active agents required for each step can be in a unit amount, or in a stock that should be diluted prior to use. In some forms, the kit includes a supply of vessels and/or devices for aliquoting and incubation of the active agents or compositions, for example, pipettes. The kits can include printed instructions for administering the compound in a method as described above.

### EXAMPLES

### Example 1: Mapping Temporal Dynamics of Antibody Responses During COVID19 at Genomic Scale and at Single-Amino Acid Resolution

Using mRNA display, barcoded peptides/proteins can be derived from DNAs chemically synthesized or fragmented from cellular/viral/bacterial/fugal cDNA, a small portion of which form epitopes recognized and enriched by immobilized antibodies. These epitopes can be identified by sequencing their barcodes.

### Methods

### DNA Library Preparation

The mRNA display library should include wild-type or mutant epitopes (peptides/domains) derived from a particular species, such as pathogens (viruses, bacteria, fungi, etc.), mammalian cells (for autoantigens or tumor antigens), or other species (allergens). DNA encoding the target epitopes can be synthesized as a pool by 1) on-chip DNA synthesis technologies, 2) synthesis of regular oligo containing mutant cassettes, or 3) fragmented from genomic or cDNAs. The pool can contain as many as millions of DNA fragments with the upper limit determined by oligo synthesis capacity or the availability of genomic/cDNA libraries. Regarding the design of the oligo pool, synethetic DNA fragments will be linked with T7 promoter sequence, Kozak sequence and sequence encoding a peptide tag (such as DYKDDDDK (SEQ ID NO. 1) tag) at their 5′ end; and sequence encoding another peptide tag (such as Strep-tagII) at their 3′ end by PCR. The resulted double-stranded DNA will be used for generating RNA in the following steps. Examples of oligo sequences used are shown in FIG. 4.

### Preparation of Peptide/Protein-mRNA Fusion Complex

The mRNA display library preparation is illustrated in FIG. 5. The pool of double-stranded DNA generated in the previous step will be in vitro transcribed into RNA by T7 RNA polymerase. The purified RNA will be ligated with a poly-dA DNA oligo fused with a puromycin at the 3′ end with assistance of a splint sequence. After that, the ligated RNA will be purified by oligo-dT beads. The resulted mRNA will be used for in vitro translation to generate the corresponding peptides/proteins. Post-translation, high concentrations of KCl and MgCl2 should be added and incubated at room temperature for 30 minutes to facilitate the fusion between the peptide/protein and RNA. Lastly, the peptide/protein-mRNA fusion will be affinity-purified by the peptide tag using specific antibodies/binders and eluted in specific buffers. For example, commercially available Strep-tactin beads can be utilized to isolate StrepTag-II containing proteins, eluted in commercially available BXT elution buffer.

### In Vitro Transcription

The in vitro transcription reaction is set up using NEB T7 RNA polymerase with the following reagents:
10× buffer (2 μl)
25 uM dNTP (0.8 μl)
100 uM DTT (1 μl)
T7 Polymerase (2 μl)
RNase inhibitor, Murine (40 units/pl) (0.5 μl)
Template DNA (200 ng)
Nuclease-free water (Up to 20 μl)

The reaction should be incubated at 37° C. for 2 hours, followed by RNA purification using an RNA clean-up kit, such as Monarch® RNA Cleanup Kit (NEB).

### RNA-Poly-dA-Puromycin Ligation Reaction

The RNA-poly-dA-puromycin ligation reaction is set up as follows:

Poly-dA-puromycin oligo:
(SEQ ID NO. 2)
Phospho-AAAAAAAAAAAAAAAAAAAAA-spacer9-spacer9-
spacer9-ACC-puromycin.
Splint oligo:
(SEQ ID NO. 3)
Phospho-ACGATAAGGGTAGCGGCTCCAAAAAAAAAAAA.

The following components should be mixed:
RNA from previous step (200 pmol)
PolyA-puromycin oligo (240 pmol)
Splint oligo (220 pmol)
Nuclease-free water (Up to 25.5 μl)

The tube should be incubated at 65° C. for 2 minutes, and then incubated on ice for 30 seconds followed by room temperature for 1 minute.

The following components should be added directly to the tube:
10×T4 Ligation Buffer (3 μl)
T4 DNA Ligase (400 units/ul) (1.5 μl)

The tube should be incubated at room temperature for 2 hours, followed by RNA purification using an RNA clean-up kit.

The ligated RNA should be digested by Lambda exonuclease to remove the splint. The reaction should be set up as follows:
Eluted RNA from previous step
10× Lambda exonuclease buffer (5 μl)
Lambda exonuclease (1.5 μl)
Nuclease-free water (Up to 50 μl)

The reaction should be incubated at 37° C. for 1 hour, followed by RNA purification using oligo-dT beads following manufacturer's protocol, such as Dynabeads™ mRNA DIRECT™ Purification Kit (61011, Thermo).

### In Vitro Translation

The in vitro translation reaction is set up as follows:
Poly-dA RNA from previous step (250 ng; Max 6 μl)
Rabbit Reticulocyte Lysate, Nuclease-Treated (17.5 μl)
Amino acid (-methionine) (0.5 μl)
Amino acid (-leucine) (0.5 μl)
RNAse inhibitor (0.5 μl)
Nuclease-free water (Up to 25 μl)

The reaction should be incubated at 30° C. for 1.5 hours.

### Peptide/Protein-mRNA Fusion Formation Reaction

The peptide/protein-mRNA fusion formation reaction is carried out as follows:

The following components should be added directly to every 25 μl) in vitro translation reaction.

| | |
|---|---|
| 1M MgCl₂ | (2 μl) |
| 3M KCl | (6 μl) |

The reaction should be incubated at room temperature for 30 minutes or −20° C. overnight.

### Peptide/Protein-mRNA Fusion

The Peptide/protein-mRNA fusion purification reaction is carried out as follows:

The reaction of previous fusion formation (33 μl) will be diluted by Strep Wash buffer (100 mM Tris-HCl pH 8.0, 150 mM NaCl, 1 mM EDTA, 0.5% TritonX-100) to 100 μl. Then, 5 μl of MagStrep Type 3 XT Beads (IBA Lifesciences) should be added and incubated at room temperature for 3 hours or 4° C. overnight. The beads can then be retained by magnet and washed by Strep Wash buffer for 3 times and Strep Wash buffer without Triton X-100 once. The peptide/protein-mRNA fusion bound by the beads should be eluted in 5 uL BXT buffer (IBA Lifesciences) at room temperature for 30 minutes.

cDNA Synthesis to Generate Peptide/Protein-mRNA-cDNA Fusion Complex

After elution, the peptide/protein-mRNA fusion should be subject to reverse transcription to synthesize cDNA, hence a peptide/protein-mRNA-cDNA fusion complex (“Fusion Complex” in short in following steps) can be formed.

### Reverse Transcription Reaction

The following reverse transcription reaction should be set up.
Elute from previous step (in 1×BXT buffer; 5 μl)
MgCl2 (2 μl)
0.1 M DTT (1 μl)
RNase OUT (0.25 μl)
SUPERSCRIPT III RT enzyme (200 U/pL, #18080-044, Life Technologies; 0.125 μl)
10 mM dNTP (0.5 μl)
Display-amp-R (10 μM 2.5 μl)
Nuclease-free water (Up to 10 μl)

The reaction should be incubated at 42° C. for 1 hour. After the reaction, a small proportion should be saved as input for NGS analysis.

Immuno-Capture of mRNA-Display Epitope Library

Capture of Antibody should be carried out as follows.

### Coating

Protein binders that can be used to purify specific class or subclass of antibodies shall be coated or bound on the surface of multi-well plates (such as 96-well plate) or beads.

### Blocking

Blocking buffer containing a mixture of detergent, proteins, RNA and DNA (such as 0.1% Tween-20, 5% bovine serum albumin, 1% fish gelatin, 40 pg/ml yeast tRNA and 40 pg/ml salmon sperm DNA in phosphate-buffered saline (PBS)) should be used to cover all the inner surface of the wells or beads to prevent non-specific binding during the following steps. The RNA and DNA should be not overlapping with the DNA sequences of epitopes, in order to avoid contaminating the final sequencing data.

### Antibody Capture

Antibody capture is carried out as follows:

Body fluid of infected animals or human subjects containing antibodies should be added into the wells and incubated at room temperature or 4° C. for 4 hours or overnight, so that specific (sub-)types of antibodies can be captured by the protein binders.

### Washing

After capturing, the plates should be washed extensively with wash buffer containing high concentration of detergent (such as 0.5% Triton-X100 and 0.1% Tween in phosphate-buffered saline) in order to remove non-captured proteins, particularly the proteases and nucleases in body fluid.

### Immuno-Capture of Fusion Complex

The solution containing a pool of fusion complexes (from Part I) should be diluted in blocking buffer and added to the wells or beads with antibody captured from previous step, so that peptides/proteins bound by the antibodies can be immunoprecipitated. 2 hours after incubation at room temperature (such as 22° C.), the solution should be aspirated, and the wells should be washed with wash buffer for 4 times.

### Elution of Fusion Complex

The immuno-captured fusion complexes can be eluted by one of the following methods:
Cleaving F(ab)2′ of specific types of antibodies using enzymes (such as IdeZ)
Reducing agent (such as DTT)
Acidic/alkaline solutions
Heat

### PCR Amplification and Barcoding

Both the input and the eluted fusion complexes should be amplified by PCR. The primers can anneal to the constant regions at both 5′ and 3′ ends (i.e., sequences encoding tags) on the input library, while containing flanking barcodes that are used to distinguish each sample (FIG. 6).

### Preparation of Sequencing Library

Barcoded PCR product can be pooled and used to prepare sequencing library, which generally includes end-repair, dA-tailing, adaptor ligation and PCR amplification (FIG. 7).

### Results

Using this method, epitopes of purified antibodies from the serum samples of SARS-CoV-2 infected patients and pre-pandemic human sera were identified. 31 infected samples (6 samples during hospitalization, 8 samples from 1 month post symptom onset (PSO), 8 samples from 4 months PSO, 5 samples from 6 months PSO) and 4 pre-pandemic samples were included. For each serum sample, IgG was isolated using ProteinG magnetic beads, IgA using home-made peptide M and subclass IgGs using monoclonal anti-IgG1/IgG2/IgG3/IgG4 antibodies. Peptide M and anti-subclass IgG antibodies were coated on Nunc MAXISORP™ flat-bottom 96-well plates (44-2404-21, Thermo) to capture corresponding antibodies.

The DNA library for mRNA display includes more than 120,000 different oligos encoding the viral proteomes of SARS-CoV-2, common cold coronaviruses (229E, OC43, NL63, HKU1), 71 commonly seen human viruses with known subtypes or serological types, and more than 1,200 known human autoantigens. More than 20,000 different mutant viral epitopes of SARS-CoV-2 and influenza virus were also included. Each oligo encodes an epitope of 48 amino-acids in length, where 24 amino acids are overlapping with the upper- and down-stream oligos.

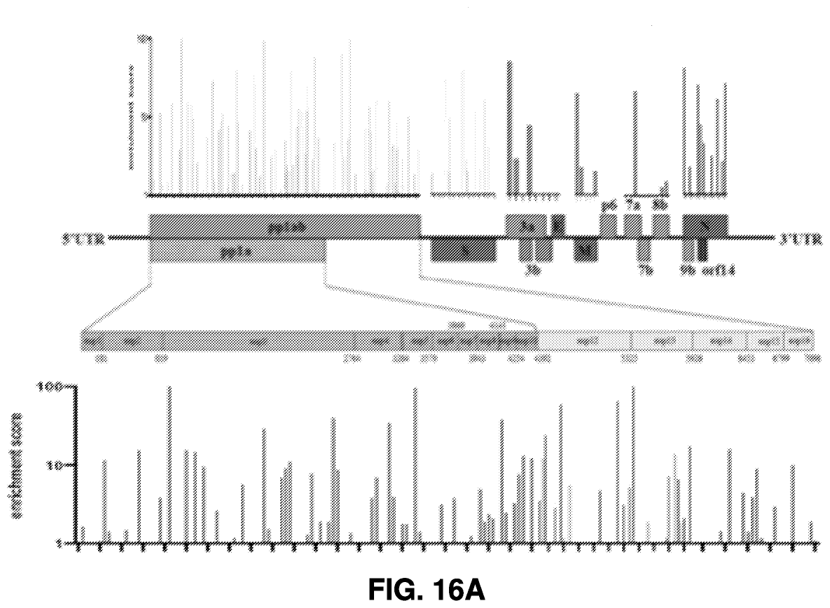

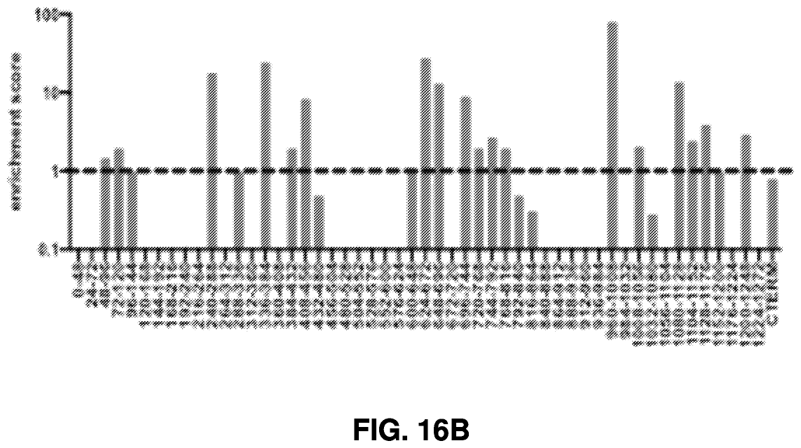

Using this library, SARS-CoV-2 epitopes were detected distributed along the whole viral genome (FIG. 16A). For spike protein (S protein) of SARS-CoV-2, the IgG epitope distribution from the dataset is highly similar with published results using peptide array (12 amino acids in length) method (FIG. 16B). Notably, the mRNA-display method detected more epitopes within the Receptor Binding Region (RBD) than the peptide array method (L1, Y. et al., Cell Rep 34, 108915, (2021)), probably because the epitopes were longer and more likely to be captured by antibodies recognizing conformational epitopes.

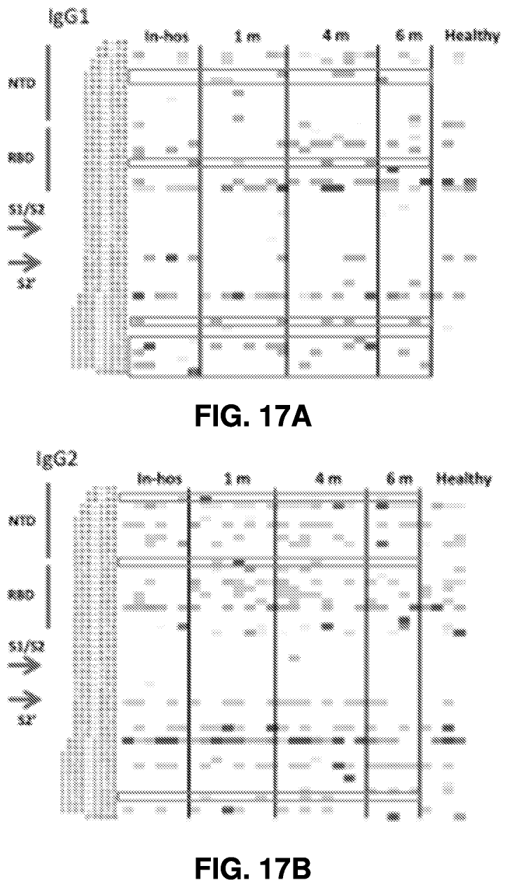

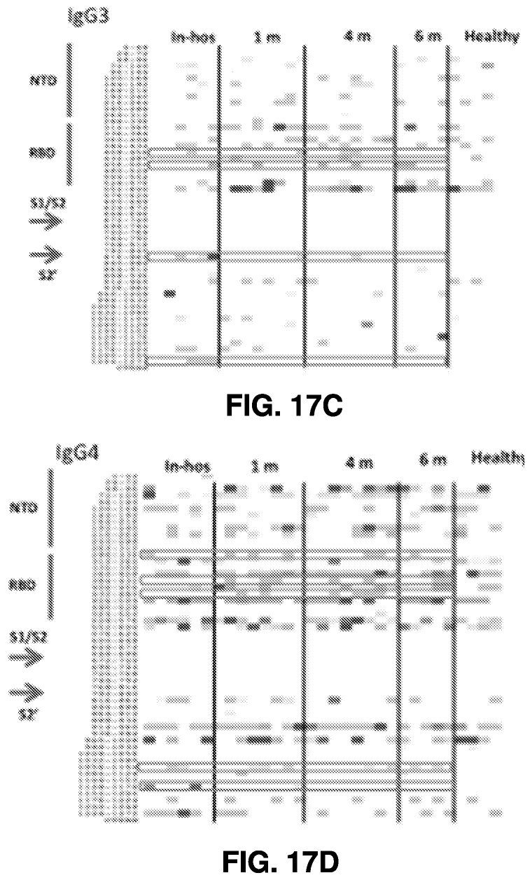

Individual subclasses are elicited by different type of antigens: antibody responses to viral and bacterial protein antigens are mainly restricted to IgG1 and IgG3 (Hammarstrom, L. & Smith, C. Monogr Allergy 19, 122-133 (1986); Linde, A. et al., Monogr Allergy 23, 27-32 (1988); Ferrante, A. et al., Pediatr Infect Dis J 9, S16-24 (1990); Visciano, M. L. et al., TJ Transl Med 10, 4, (2012)), while IgG2 is generally produced in response to carbohydrate antigens (Adderson, E. E. et al., J Immunol 147, 1667-1674 (1991); Adderson, E. E. et al., J Clin Invest 91, 2734-2743, (1993); Sanders, L. A. et al., Pediatr Res 37, 812-819, (1995)). In general, it is possible to conclude that in mice and humans IgG1 (as well as IgG4 in humans) is associated with a Th2 profile and the other subclasses are mainly associated with a Th1 profile. The epitopes recognized by different classes and subclasses of antibodies showed distinct distributions, exemplified by the epitopes on S protein (FIGS. 17A-17D). The longitudinal comparison for certain epitopes from the same patients showed different temporal dynamics of antibody responses from each subclass of IgGs. Particularly, for epitope at 384-482 position on S protein, IgG1 and IgG2 responses decayed significantly at 6 months PSO, whereas IgG3 and IgG4 were not. IgG1 and IgG2 composite the large proportion of the total serum IgGs—66% and 23%, respectively. The decay of IgG1 and IgG2 is consistent with the observation that total IgG is shown as decreased at 6 months PSO in the dataset, as well as the other published results. However, the persistence of IgG3 and IgG4 indicates that some of the immune memory might last longer than expected.

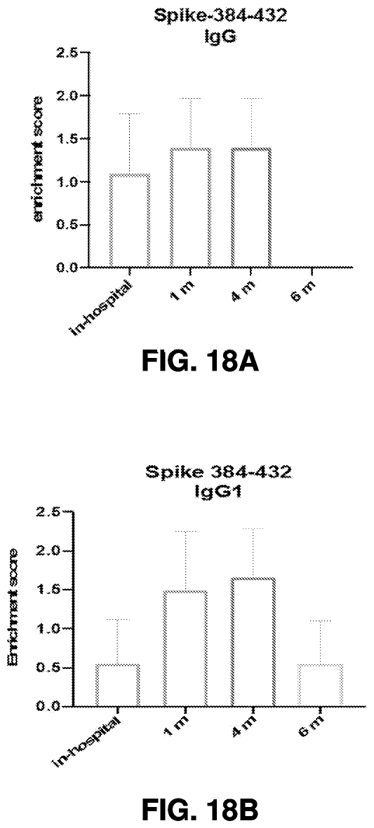

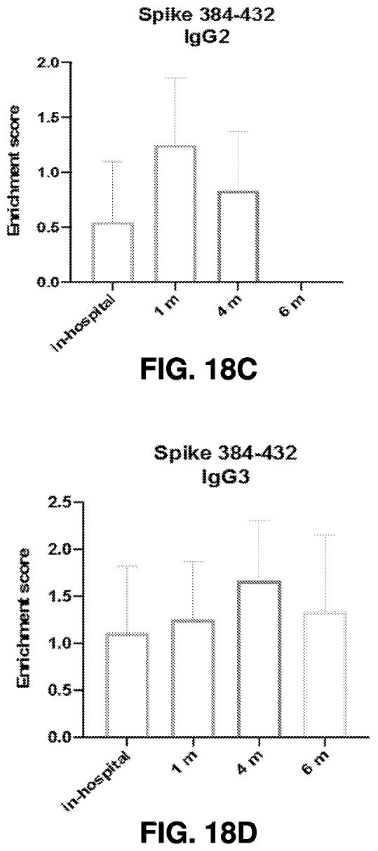

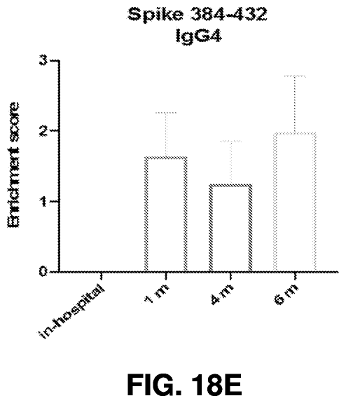

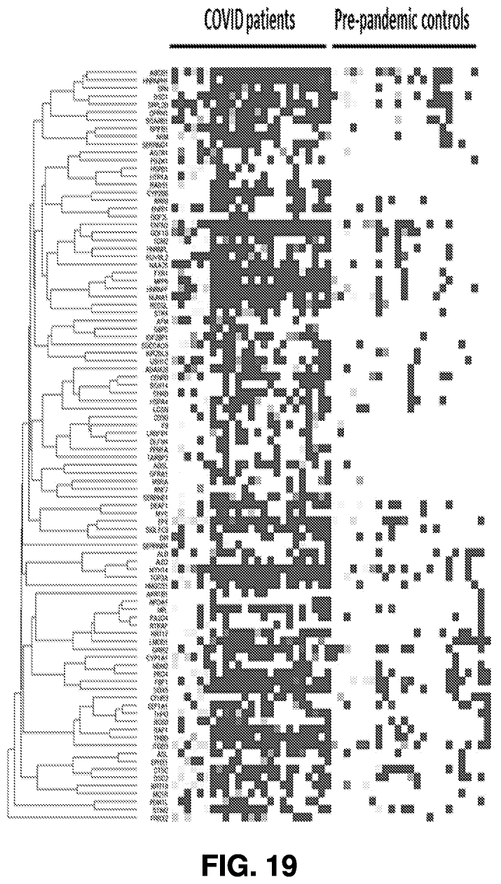

These data also comprehensively profiled the antibody responses against auto-antigens in the COVID19 patients and pre-pandemic controls. The library contains −20,000 epitopes of 1,167 human autoantigens. Based on the auto-antigen results from 25 COVID19 patients and 25 healthy controls, IgG antibody responses to 45 auto antigens are significantly higher in COVID19 patients than healthy controls (FIGS. 18A-18D). Within the 45 auto-antigens, 6 are associated with neurological disorders (Table 1) and 10 are associated with blood coagulation (Table 2). These auto-antigens might be associated with currently observed complications of COVID19, such as thrombosis and neurological symptoms, such as anosmia. These complications usually occur after the clearance of viral replication, which is consistent with the delay of immune responses against self-antigens, rather than viral antigens.

TABLE 1
Auto-Antigens associated with neurological disorders
Associated with Neurologic Disorders

CNTN2
GPI
GRIK2
HSPB1
HTR1A
IGF2BP1

TABLE 2
Auto-Antigens associated with blood coagulation
Associated with Blood Coagulation

MPL
THPO
ITGB3
THBD
F9
APOA1
SERPING1
SERPINE1
RAF1
SPN

### Example 2: Mapping Temporal Dynamics of Antibody Responses During COVID19 at Genomic Scale and at Single-Amino Acid Resolution

### Methods

The Sequencing-Linked ImmunoSorbent Assay (SLISA) set forth in FIG. 1 was used to comprehensively map the antibody profile against SARS-CoV-2 at single epitope and single amino acid level resolution. The enzyme in ELISA was replaced with a large panel of nuclear acid tags for signal amplification, which increases both throughput and sensitivity simultaneously. Differential and dynamic antibody responses targeting different regions of the viral proteome during and after infection were revealed, with epitopes in the S1/S2 cleavage sight and at the N-terminus persistently enriched even six months post symptom onset.

The library of peptide-nuclear acid fusion complexes for SLISA was prepared by in vitro transcription and translation. Briefly, single-stranded DNA encoding peptides of interest were synthesized as a pool and converted to double-stranded DNA by PCR. RNAs were transcribed in vitro from the DNA and ligated with poly-dA oligo conjugated with puromycin. Peptides were then synthesized from the RNAs by in vitro translation, where each RNA was fused with the corresponding peptide due to the presence of puromycin. Next, cDNAs complementing with the RNAs were synthesized by reverse transcription. The library of peptide-mRNA-cDNA fusion complexes was ready for immunoprecipitation. In parallel with the preparation of peptide-mRNA-cDNA fusion complexes, antibodies from body fluids (such as sera) from human subjects or animals were captured on solid surface (such as ELISA plates or beads) by specific antibody capture proteins, e.g. protein G for IgG, peptide M for IgA. After washing off other components in the body fluid, the fusion complex library was incubated with the solid surface. The epitopes recognized by the antibodies will be captured and remained on the surface. Finally, the epitopes were eluted; the cDNAs conjugated on the epitopes were amplified by PCR and analyzed by next generation sequencing.

### Results

### Mapping Antibody Responses Against SARS-CoV-2 Proteome

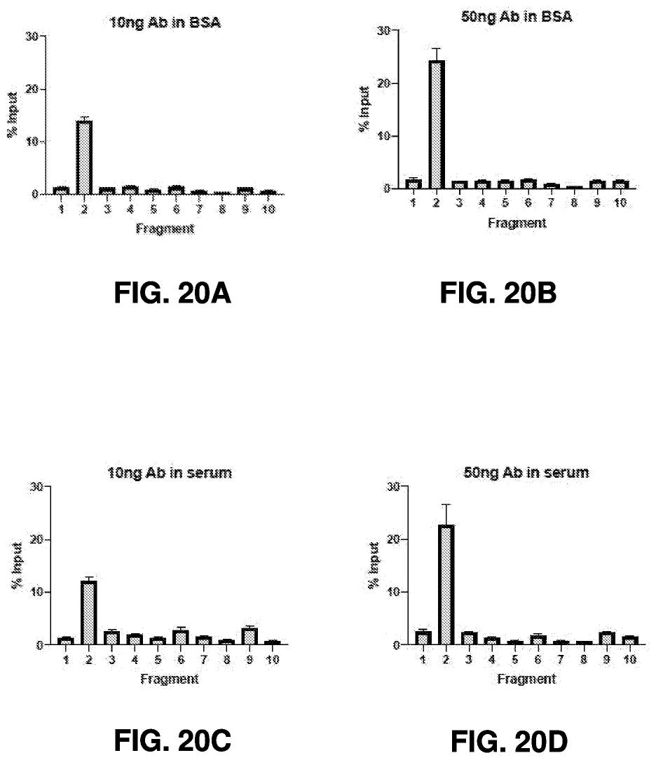

To demonstrate that SLISA can identify the epitope that an antibody binds to, a library including 10 fragments of SARS2-CoV-2 Nucleocapsid protein was first generated. Monoclonal antibodies recognizing the N-terminals of N protein, diluted in BSA or human serum respectively, were immobilized and then incubated with the fusion complex library. After elution, the enrichment of 10 fragments of SARS-CoV-2 proteins bound by 10 ng, or 50 ng, monoclonal Ab diluted in BSA or pre-pandemic human sera was examined by qPCR. As expected, the N-terminal domain of N proteins were highly enriched whereas other domains were not (FIGS. 20A-D). Furthermore, higher amount of antibody (50 ng) resulted in higher enrichment than lower amount (10 ng), and dilution in human serum didn't interfere the enrichment.

A DNA oligo library was then synthesized as a pool of 4 groups:
(1) peptides covering the proteome of representative strains of human coronaviruses (HCoV) and SARS-CoV-2 variants reported until June 2020;
(2) single amino acid variants of 78 SARS-CoV-2 and common cold HCoV epitopes, based on the results of group 1 peptide;
(3) peptides covered all human protein auto-antigens documented in database AAgAtlas; and
(4) peptides covering the genomes of commonly seen human viruses, including genotypes/serological types.

Each peptide was 48 amino-acid long spanning the proteomes, with 24 amino-acid overlaps. In total, the library contains about 189,000 different epitopes.

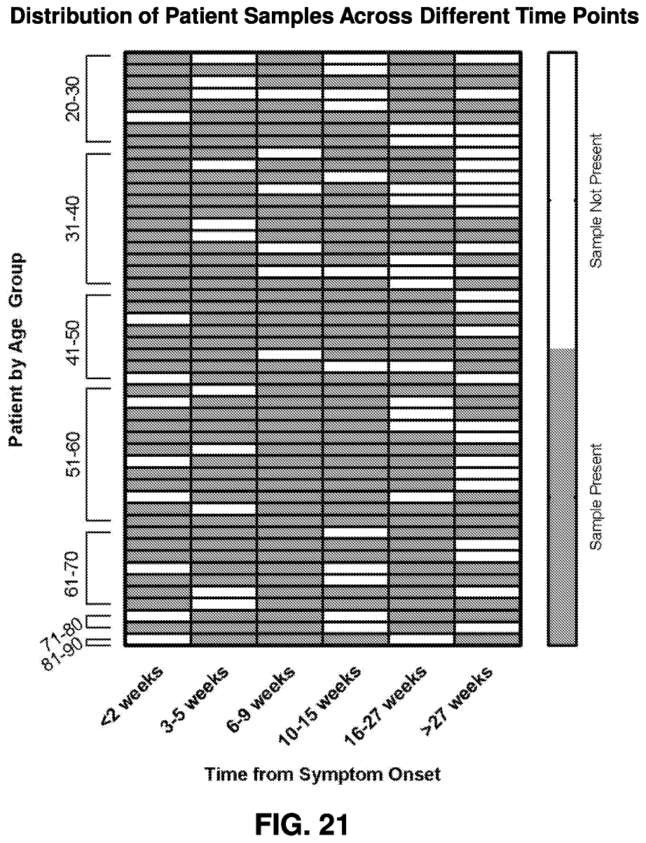

Sera from 55 COVID19 patients was included in the study. Blood samples were taken at multiple points for each patient, spanning the time period from 1 week to 6 months after symptom onset (FIG. 21).

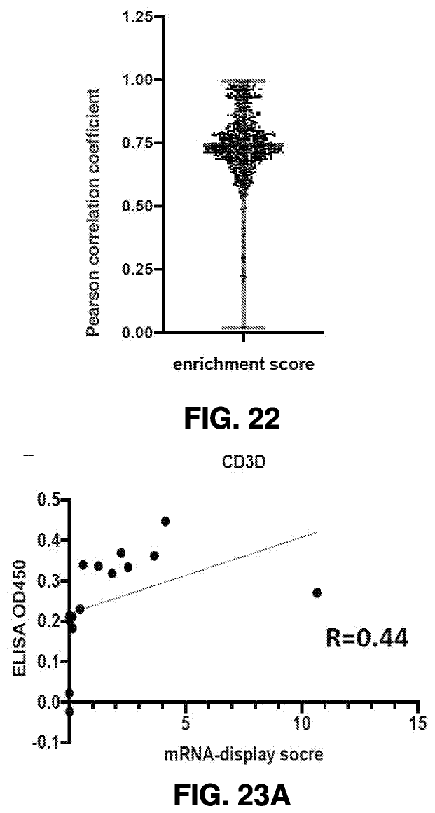

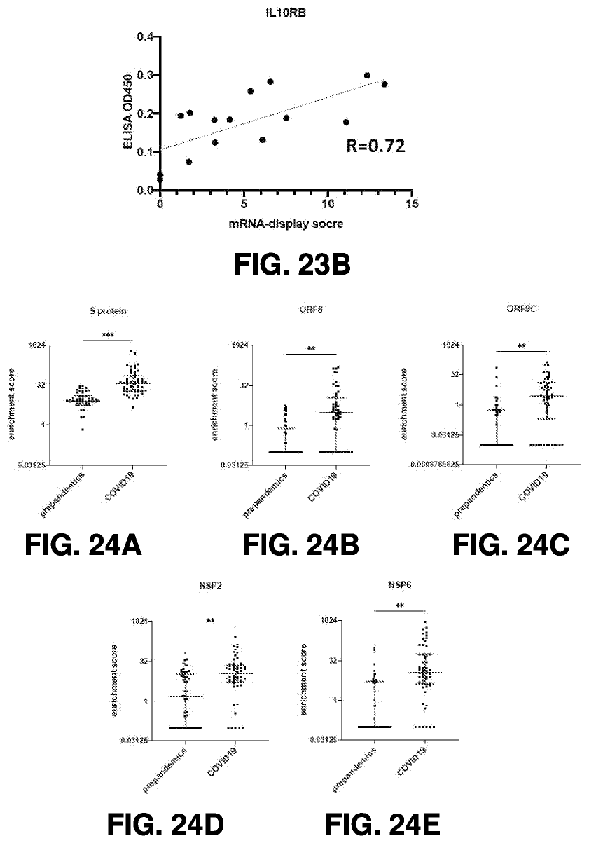

There was no significant gender bias in each age group in the patient cohort. Among the patients, 5 of them were admitted into intensive care unit (ICU) and the rest 48 were not. There were also 53 age and gender matched random pre-pandemic sera samples being included as controls. SLISA was performed on IgG from the sera samples. Enrichment score was calculated on each epitope to represent the binding strength of the epitope by serum antibodies. The median of correlation co-efficient between technical replicates and the calculated enrichment score on the same serum sample was 0.75 (FIG. 22). Also, the SLISA enrichment score showed good correlation with OD450 ELISA on both the selected antigens from human sera samples (anti-CD3D auto-antibody, and anti-IL10RB auto-antibody), respectively (FIGS. 23A-23B).

### Using SLISA to Map Antibody Responses Against SARS-CoV-2 Proteome

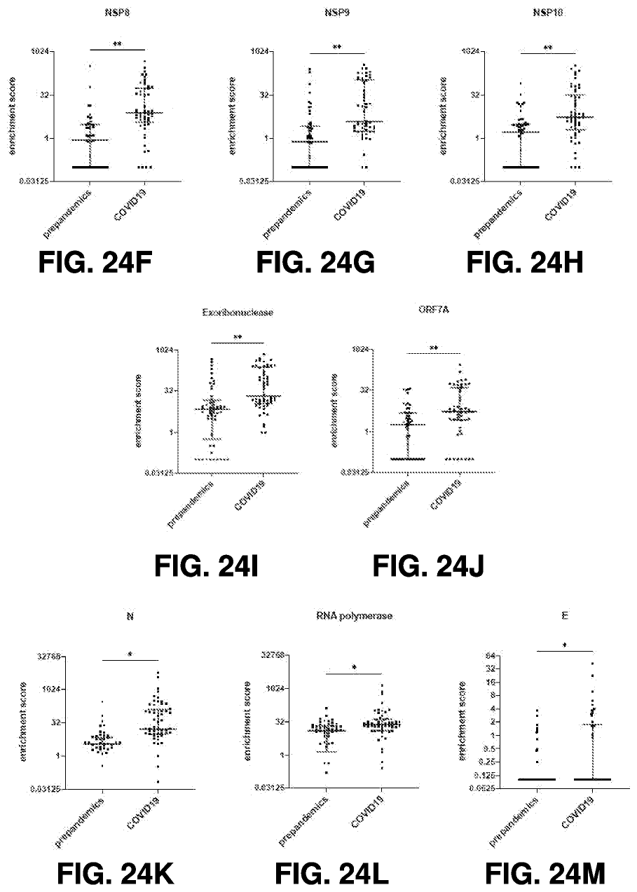

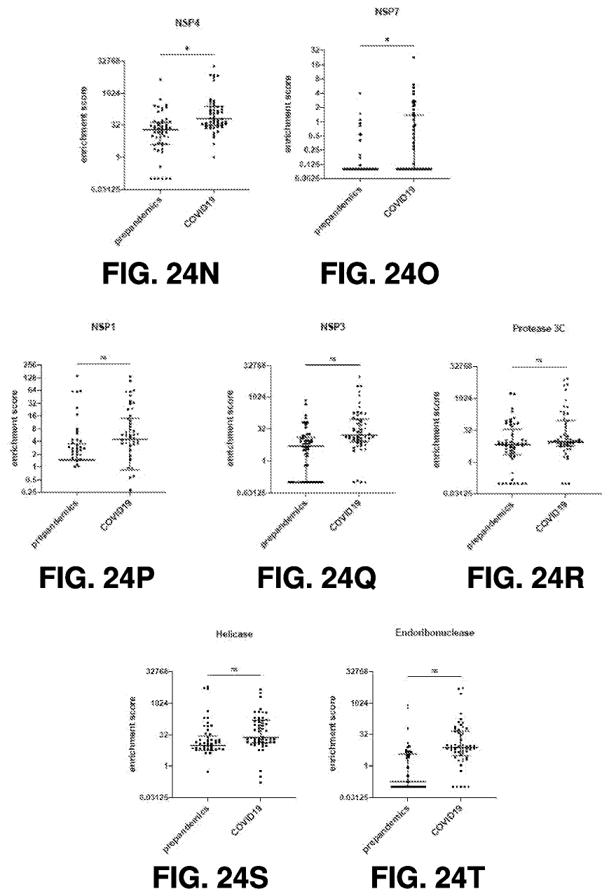

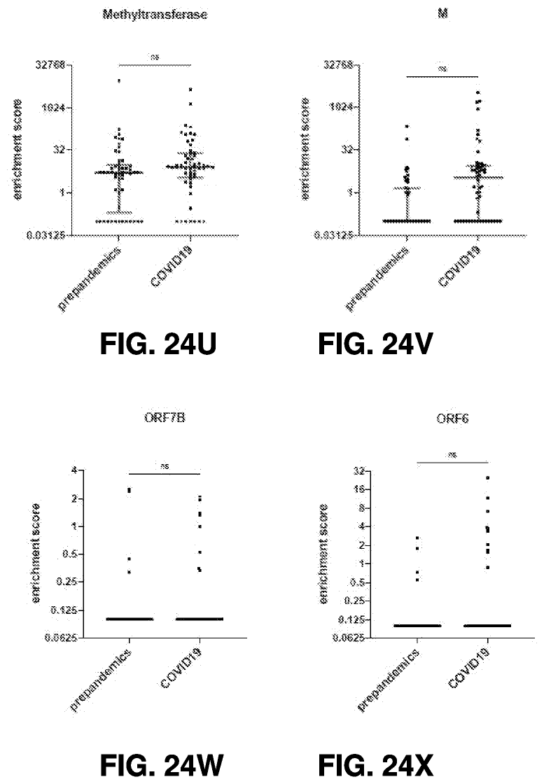

The enrichment score of all peptides on each protein of SARS-CoV-2 in both COVID19 patients and pre-pandemic controls was first calculated. The SLISA enrichment score of each viral protein was the sum of enrichment scores of all peptides. For COVID19 patients, the maximum SLISA enrichment score across multiple time points was selected and plotted (FIGS. 24A-24X). Consistent with previous reports, IgG responses in COVID19 patients against one group of viral proteins, including S, N, ORF8, ORF9C etc. were significantly higher than that of pre-pandemic controls. This indicated that COVID19 induced antibody responses against these viral proteins either by de novo generation or by enhancing the pre-existing antibody responses against common cold HCoVs.

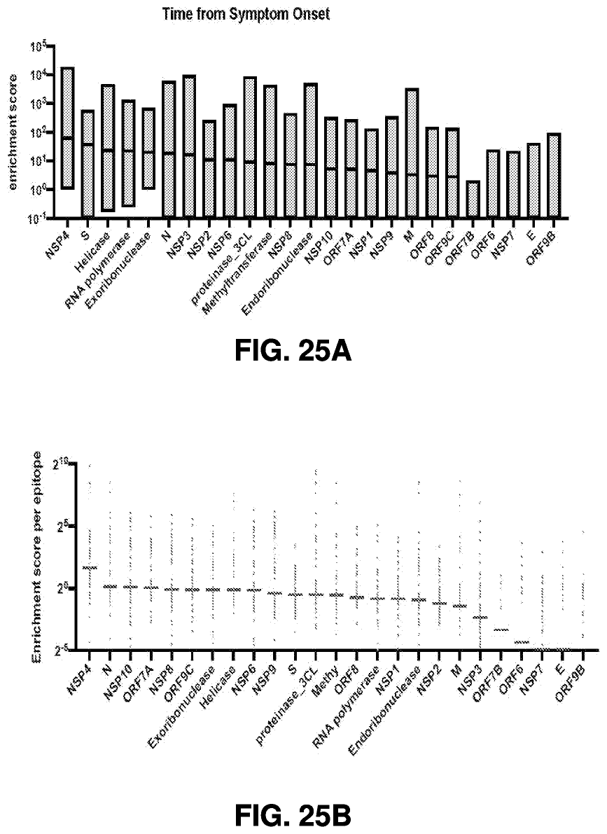

The SLISA enrichment score for each viral protein of SARS-CoV-2, and average SLISA enrichment score of each peptide on SARS-CoV-2 were analyzed (FIGS. 25A-25B). Among the viral proteins, NSP4 showed the highest enrichment score for both the sum of all peptides and average per epitope, indicating the high immunogenicity of this non-structural protein. S and N proteins, the two antigens that have been commonly used for antibody detection, also showed high enrichment scores in the results.

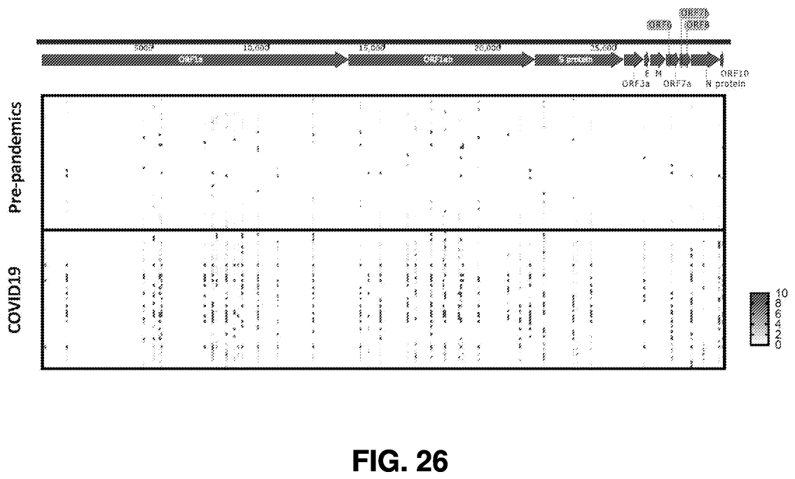

Among the 409 peptides spanning SARS-CoV-2 proteome, 45 peptides that are significantly higher enriched in COVID19 patients than in pre-pandemic controls (p<0.001, fold change >20) were identified (FIG. 26). These epitopes fall into ORF1a, ORF1ab, S, M, ORF8, N and ORF9C open reading frames, suggesting that multiple structural and non-structural proteins of SARS-CoV-2 can stimulate specific host antibody responses.

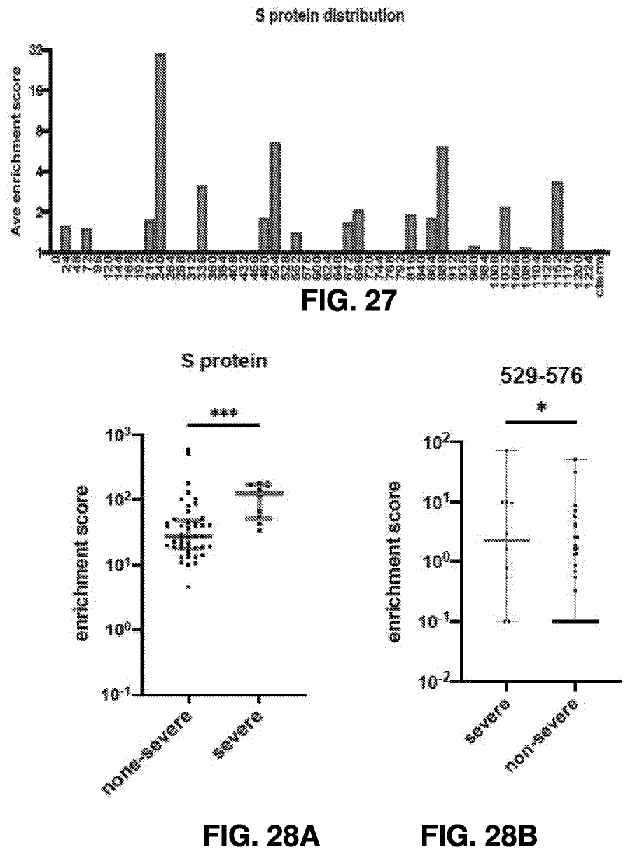

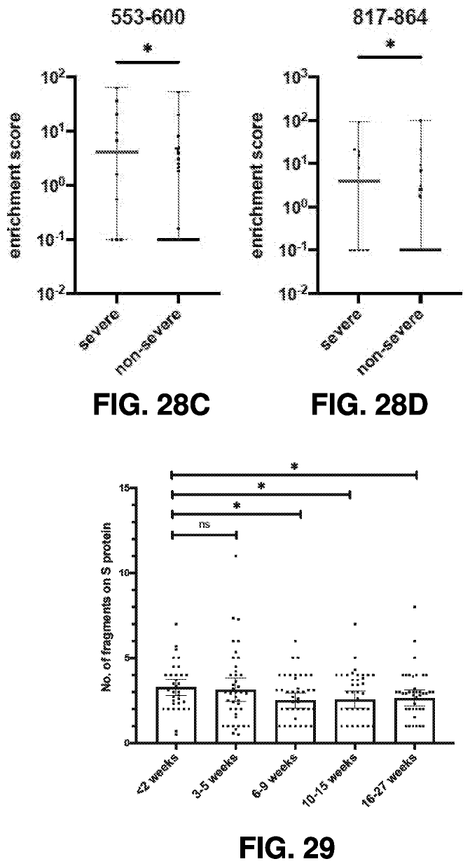

Distribution of enriched peptides across S protein of SARS-CoV-2 as also assessed. The epitope distribution on S protein in the dataset is highly overlapping at the S1/S2 and S2/S2′ cleavage sites with previous report using VirScan, a phage display technology (FIG. 27). However, the method identified more epitopes within the receptor binding domain (RBD), probably due to the better peptide expression using the mammalian translation system than using phages in E coli. Enrichment score of S protein in non-severe and severe patients was assessed. Several reports have shown that antibody titer against S protein correlates with disease severity, which is also shown in the dataset (FIG. 28A). Furthermore, among all the peptides within S protein, enrichment scores on 3 peptides, peptide 529-576, peptide 553-600 and peptide 817-864, were significantly higher in severe patients than non-severe patients (FIGS. 28B-28D). Two of them are at the C-terminus region of RBD (319-541). It was reported that antibodies against this region were non-neutralizing (website medrxiv.org/content/10.1101/2020.10.08.20209114v1). It is possible that these non-neutralizing antibodies induce antibody dependent entry (ADE) thus causing more severe infection. The 3^{rd} peptide, 817-864 locates next to the S2/S2′ cleavage site (815/816).

### Antibody Responses Against SARS-CoV-2 Keep Changing During Acute and Convalescent Phases of COVID19

To investigate the temporal dynamic changes of antibody responses against SARS-CoV-2, the positive rate of each peptide among the 55 COVID19 patients at each time slot were plotted, and the results showed that the temporal dynamics of positive rate of epitopes vary dramatically at difference sites on the S protein. Peptide 672-720, covering the S1/S2 cleavage, remained high positive rate even 6 months after symptom onset. Notably, this region has low sequence similarity with spike proteins of common cold coronaviruses, and is among the 45 SARS-CoV-2 specific epitopes that were identified, indicating that COVID19-specific antibody responses can last fairly long. In addition, the enrichment scores for relatively conserved regions, such as 865-936 and 1033-1080, sustained high positive rate through the recovery, possibly because of the crosstalk of pre-existing anti-HCoV antibodies. The number of enriched peptides on S protein in individual patient during listed time slots was plotted, with each dot representing one patient. Overall, the number of positively enriched peptides start decreasing significantly from 6-9 weeks after symptom onset (FIG. 29).

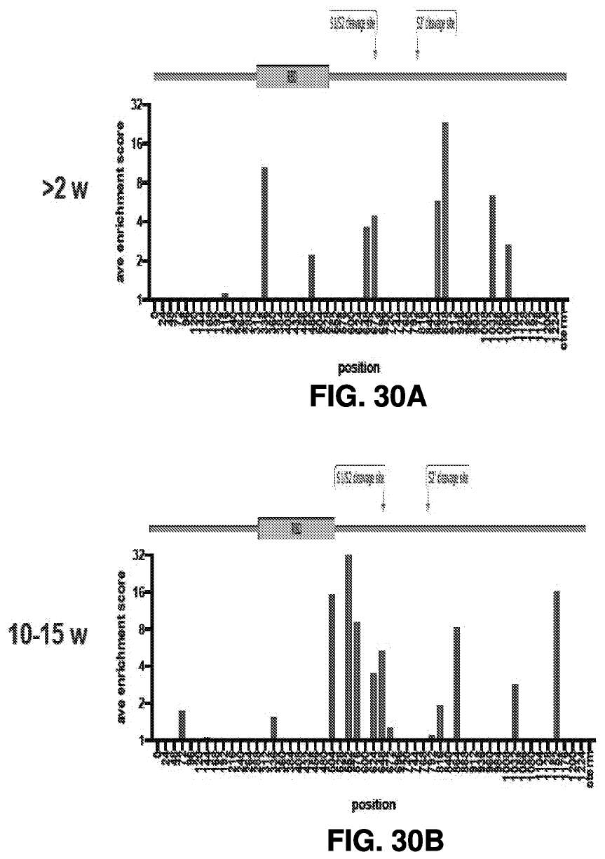

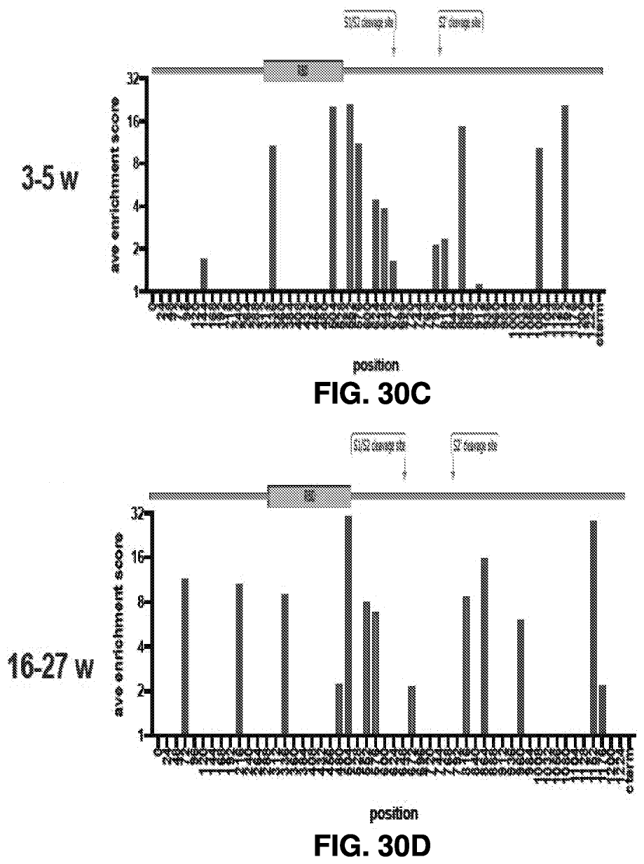

The average quantitative enrichment scores of each peptide across S protein from samples in each time slot was then mapped (FIGS. 30A-30D). Across multiple time points, the distribution of epitopes always clustered at N terminus, RBD region, S1/S2 cleavage sites, S2/S2′ cleavage site and C-terminus. However, the enrichment score for each peptide was constantly changing. As expected, if examining the enrichment of each individual patient, the enrichment score of each epitope on S protein had distinct temporal pattern. Furthermore, the location of long-lasting peptides (defined as >50% of enrichment score of the peak time point) presented as a subject-dependent manner.

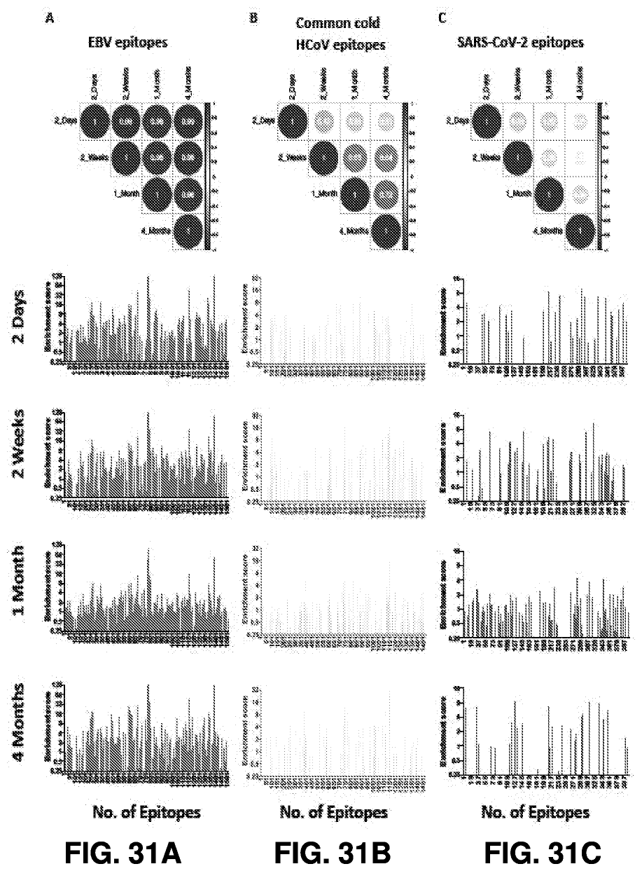

Pearson correlation co-efficient between peptide enrichment scores of EBV, common cold HCoV or SARS-CoV-2 at multiple timepoints for one individual patient were plotted, together with enrichment scores of each single epitope on the sample collected at indicated time point for the same patient (FIGS. 31A-31C). The correlation co-efficient of epitopes on the proteome of Epstein-Barr virus (EBV), a virus establishing persisting infection, was very high among multiple time points for the same patient (FIG. 31A), suggesting relatively stable antibody responses against persisting infection. The antibody responses against SARS-CoV-2 epitopes, however, showed weak correlation between different time points, indicating constantly changing antibody responses during recovery (FIG. 31C). Analyzing the data of peptides of common cold HCoVs, weak correlation at the acute phase of SARS-CoV-2 infection was observed, then the correlation coefficient was increased during long term recovery (FIG. 31B). This may be because the newly established antibody responses at acute phase of COVID19 has some cross-reactivity.

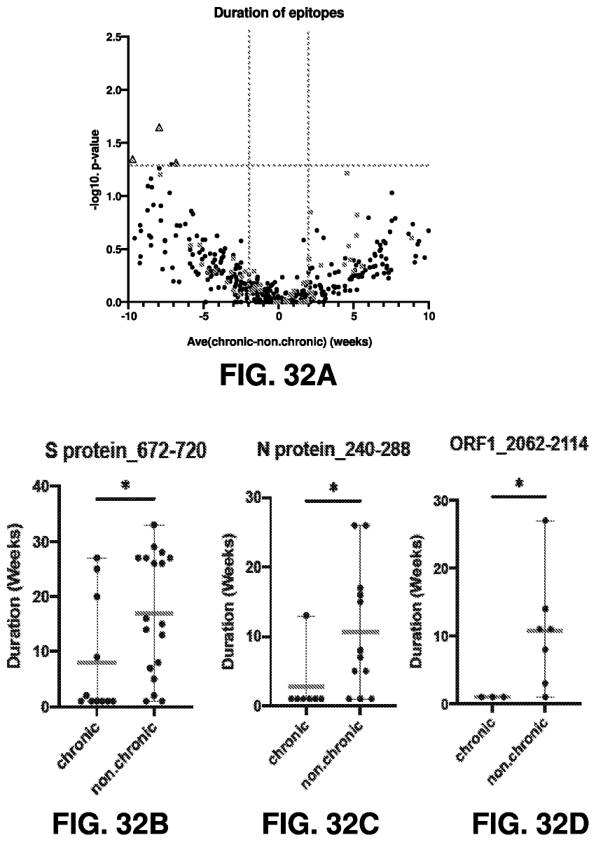

It is known that pre-existing chronic diseases are correlated with disease severity during COVID19. However, the temporal dynamics of antibody responses in these patients have not been comprehensively investigated. The results showed that for majority of peptides, patients with chronic diseases, such as hypertension, diabetes, hyperlipidemia, COPD, chronic liver disease, chronic heart disease, etc., did not show significant difference compared to patients without these conditions (FIG. 32A). Duration of 3 peptides with significantly differential duration in patients with chronic diseases and patients without chronic diseases were plotted, with each dot representing the duration for one patient. On the contrary, against 3 peptides in S, N and NSP3, respectively, patients with chronic conditions showed significantly shorter duration for antibody responses (FIG. 32B-32D). These included antibody against 673-720 of S protein, in particular, which covers the S1/S2 cleavage site (685/686), and several studies have shown that antibody against this region could be neutralizing.

### Antibody Responses Against Single Amino Acid Variants

Sera from human subjects contain hundreds of thousands of species of antibodies against a great number of epitopes, and it has been a challenge to profile the behavior of all individual antibody species. An epitope library encoding variants changing every single residue to other 19 amino acids, which covers all possible single amino acid mutations was therefore established. This library would enable investigation of the specificities of thousands of subsets of antibodies and give a comprehensive pictures of how antibody responses against viral peptides with newly emerged mutations or potential mutations in the future.

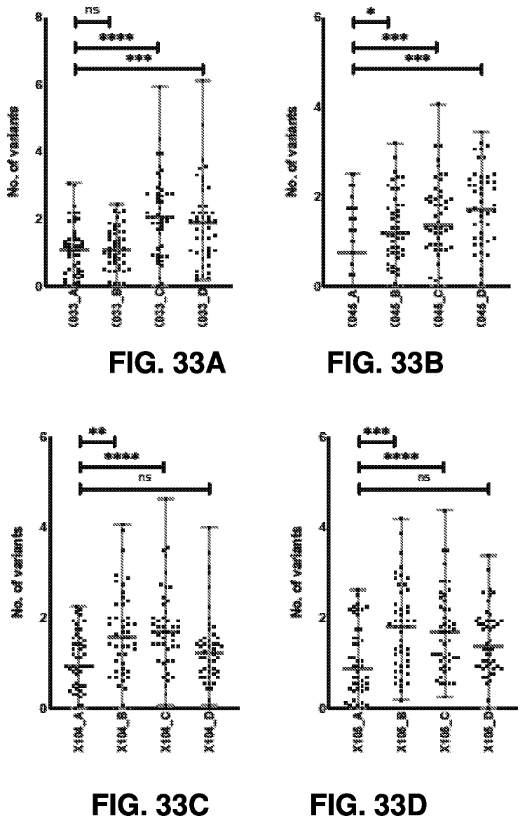

The coverage of epitope variants on S protein at each amino acid position on 4 patients at 4 time points is shown in Figure. The temporal dynamics of coverage showed different patterns in different patients. One example is peptide 649-696 covering S1/S2 cleavage site (685/686). The number of variants at each amino acid position within S protein 649-696 peptide that can be bound by multiple time points was determined and plotted for each patient (033; 045; 104 and 105), with one dot representing one amino acid position (FIGS. 33A-33D). Patients 033 and 104 showed broadest antibody responses against variants at 3 months (FIGS. 33A and 33C), whereas patient the broadest for patient 045 is at 6 months (FIG. 33B) and patient 105 is at 1 month (FIG. 33D).

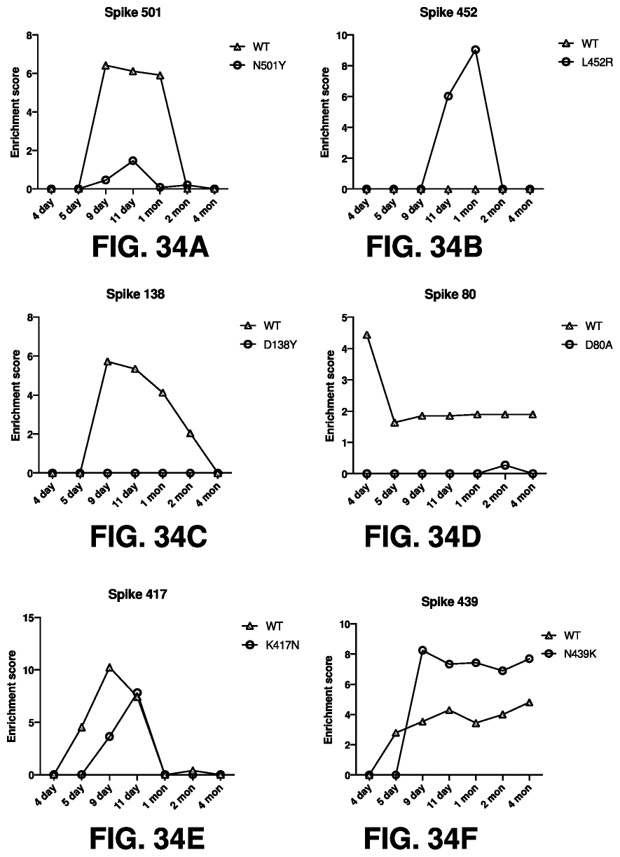

In addition to the temporal dynamics of broadness, the binding strength of antibodies against each variant also changed dynamically, as exemplified by patient 033. In particular, for variant P681R, which is carried by currently prevalent strain B.1.617.2 (also known as Delta strain), the binding strength on patient 033 kept increasing from 2 weeks to 6 months compared with wide-type variant. The binding strength of all possible variants within the RBD domain were also plotted (331-531 on S protein). This dataset also revealed the dynamics of antibody responses binding to epitopes of mutant SARS-CoV-2 strains (FIG. 34A-34F).

### Antibody Responses to Autoantigens

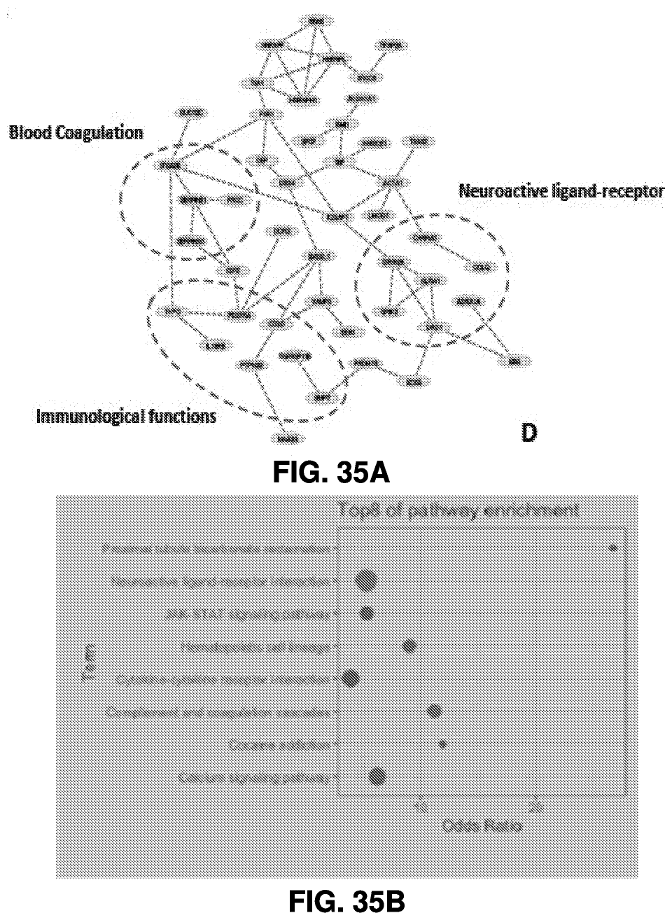

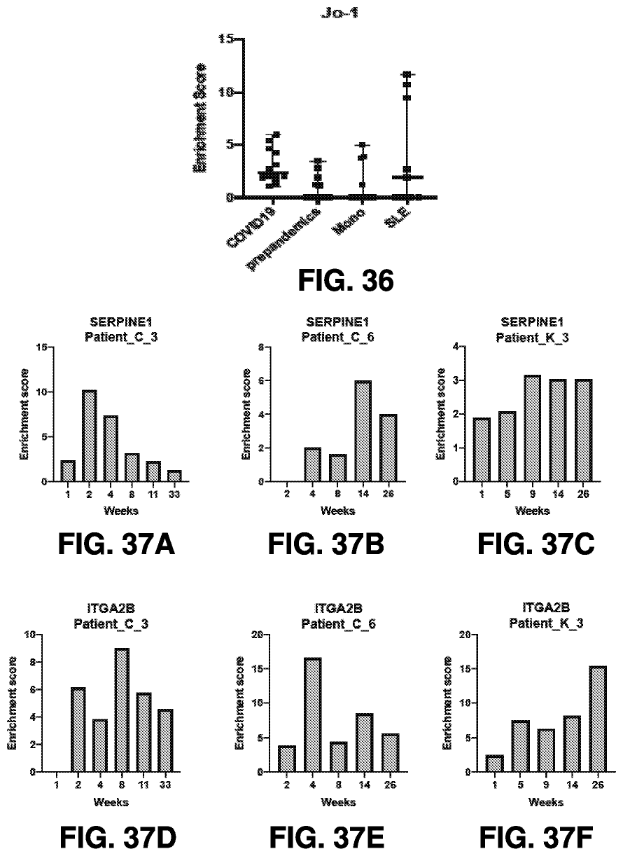

In addition to viral proteomes, peptides covering all 1160 known autoantigens were also included in the SLISA library. Several studies have reported that COVID19 patients showed symptoms that may be related to autoimmune responses. These symptoms may appear transiently during infection, but many persists during and after recovery. Furthermore, autoantibodies have also been shown to be enriched in patients suffering from severe COVID19. In this study, peptides covering all known 1164 auto-antigens were included in the SLISA library, so that SLISA allows to comprehensively evaluate the specificity and temporal dynamics of antibody responses to all previously known auto-antigens that may be involved in the pathogenesis of COVID19. By comparing the enrichment scores between COVID19 patients and pre-pandemic controls, 73 autoantigens that are more associated with COVID19 were identified. These 73 auto-antigens significantly enriched into pathways related to neuroactive ligand-receptor interaction, complement and coagulation cascades and cytokine-cytokine receptor interaction, which were reported to be COVID19 associated manifestations (FIG. 35A-35B). To compare the auto-antibody responses between COVID19, auto-immune diseases and other acute infections, SLISA were also performed on sera from 9 systemic lupus erythematosus (SLE) patients and 12 primary infectious mononucleosis patients. From the SLE patients, significantly higher enrichment was observed in SLE patients on well-known autoantigens than other groups, such as Jo-1 (FIG. 36). Data were extracted on 12 COVID19 patients with age and gender matching with the mononucleosis patients, and compared the enrichment of the 73 COVID19 associated autoantigens. Autoantigens were significantly higher enriched in the sera of COVID19 patients than mononucleosis patients, whereas no significant difference were observed for autoantigens, indicating that SARS-CoV-2 infection is associated with a specific group of auto-antibodies. When the responses to the 73 COVID19 associated autoantigens between COVID19 and SLE patients was compared, the majority of these autoantigens showed no significant difference on enrichment score. This suggest that auto-immune disease SLE is associated with overall high auto-antibody responses, but the auto-immune responses associated with COVID19 is elevated but with distinctive patterns.

Next, the temporal dynamics of the auto-antibody responses in COVID19 patients were examined. A few autoantigens were selected and their dynamics plotted on multiple patients as examples (FIGS. 37A-37F). For example, enrichment scores of Serpin Family E Member 1 (SERPINE1), an important inhibitor of fibrinolysis, presented very different dynamic patterns on different patients—either peak in the acute phase or in the recovery phase (8-15 weeks, FIGS. 37A-37F). Next, in order to give an overview of the dynamics of all autoantigens on all patients, for a particular autoantigen how many patients reach to peak within each week was calculated. The results showed that for most of the autoantigens the peaks clustered between 2 to 5 weeks, but some auto-antigens that presented clusters of peaks at 15-16 weeks were observed, such as EEF2K and SAG.

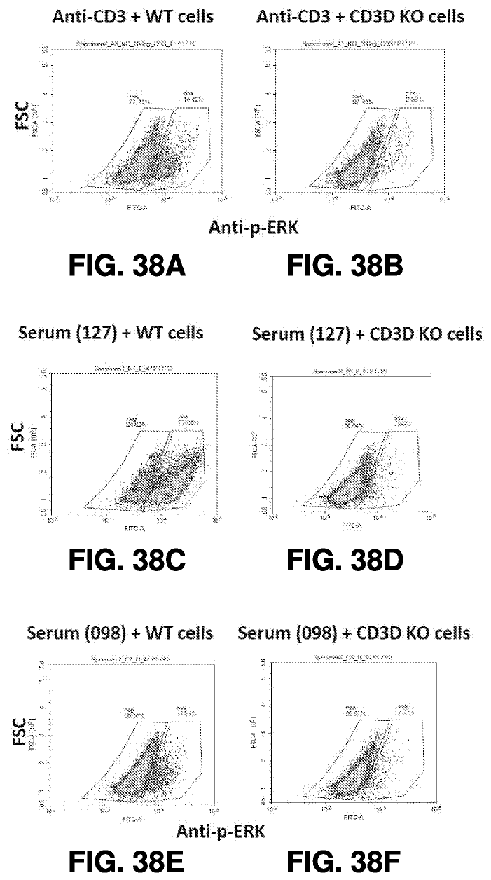

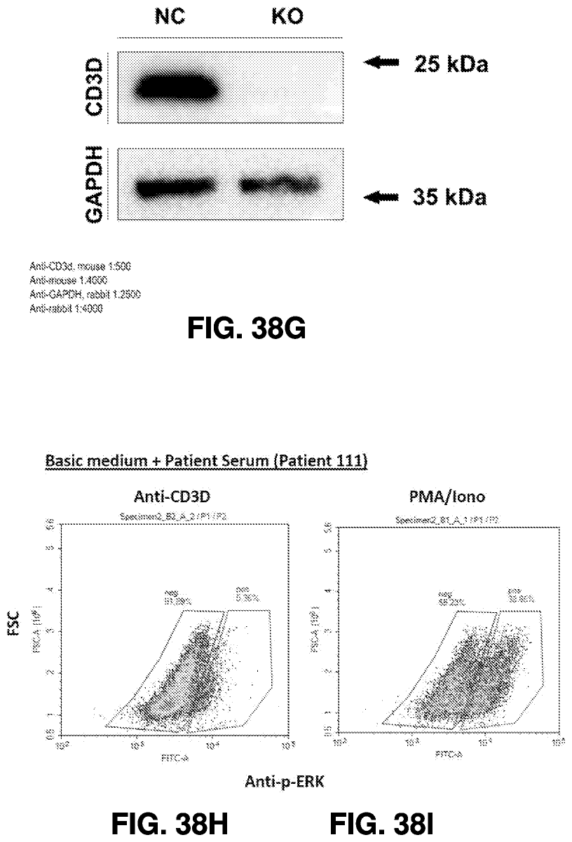

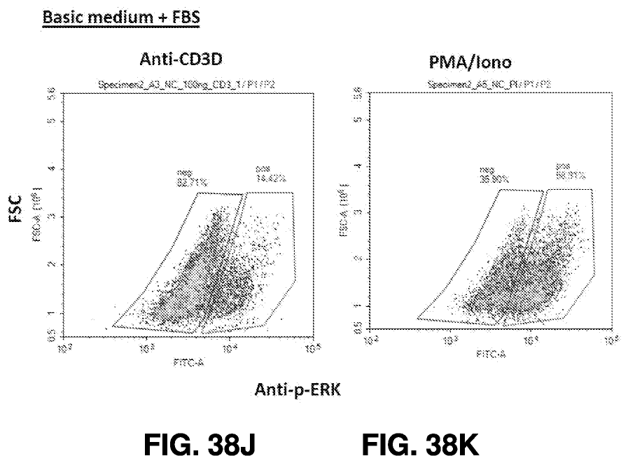

Several studies have reported that auto-antibodies against components of the innate and adaptive immune system, such as IFN, CD3E and complements could block the immune responses after infection. Sera from 6 patients which contained CD3D auto-antibodies revealed by SLISA were tested and verified by ELISA. To test whether the auto-antibodies could block T cell activation, Jurkat T cells were pre-incubated with each serum sample, treated T cells with mouse anti-CD3 antibody (clone: OKT3) or phorbol 12-myristate 13-acetate (PMA)/Ionomycin, a combination of cell permeable PKC activator (PMA) and calcium ionophore (Ionomycin) with well-known activity to stimulate T cells. Among the 6 serum samples, 1 sample was shown to block ERK phosphorylation mediated by OKT3 antibody whereas not blocking PMA/Ionomycin's impact on pERK. This suggested that the serum contained element inhibiting OKT3's function, which binds to CD3 at the cell surface, but not inhibiting ERK phosphorylation induced by intracellular stimulation. To examine whether the auto-antibodies themselves could activate T cells, the Jurkat T cells were treated with or without knocking out CD3D with serum sample (FIGS. 38A-38K). Flowcytometry staining phosphor-ERK (pERK) was used as readout for Jurkat T cell activation. Among six patient samples, two sera samples could induce ERK phosphorylation on wildtype Jurkat cells but not CD3D KO cells. The data show that there the sera samples contain antibodies recognizing CD3D and activities that can stimulate ERK phosphorylation in T cells mediated by CD3D.

### DISCUSSION

In this study, a SLISA platform was established and antibody responses of COVID19 patients were mapped against peptides of SARS-CoV-2, commonly seen human viruses and known human auto-antigens. The data comprehensively revealed the temporal dynamics of antibody responses during SARS-CoV-2 infection and convalescence at genomic scale and at single-amino acid resolution.

The results showed that it is prevalent in the patient cohort that antibody responses against SARS-CoV-2 peptides kept changing even months after recovery form COVID19. Two explanations causing the vivid dynamics are discussed. Residual viruses or viral antigens keep stimulating the evolution of B cell clones: previous studies reported cases that B cell clones keep evolving because the residual viruses. Multiple studies have provided evidence that viruses or viral antigens may remain in the human body for a long period of time after COVID19 recovery (Citation), some of which resulted in the relapsing on RT-qPCR test. Alternatively, different clones of the plasmablasts and long-lived bone marrow plasma cells (BMPCs) behave distinctly at each time point: for example, the life span of plasmablasts is usually months and after that, the major recourses of serum antibody come from BMPCs.

73 COVID19 associated auto-antigens were identified from 1160 known human auto-antigens. These auto-antigens enrich into neurological, immunological and coagulation pathways, which are related to COVID19 complications such as anosmia, fatigue, lymphopenia and thrombosis. The incident rate of auto-immune complications in Hong Kong and other Asian areas is generally low and none of the patients in this cohort showed severe disease symptoms. Furthermore, the cohort contains samples from multiple time points, but the total number of patients is small. Therefore, it is difficult to conclude whether these auto-antigens are correlated with one of the COVID19 complications. However, the data suggest that the antibody responses against auto-antigens is increased even in mild COVID19 patients.

This library also assays the antibody coverage to all possible simple mutations that SARS-COV-2 can acquire at these locations, which is particularly important, considering the continual acquisition of mutations on the SARS-COV-2 genome and the efficacy of vaccination. Especially as more vaccines are being developed, specific information regarding the epitope targets is needed to discern which epitopes generate more long-lasting antibodies and whether these antibodies can tolerate mutations in their recognized epitopes.

The SLISA platform is broadly applicable to profile antibody responses during infection, vaccination, transplantation, allergy, auto immune diseases and cancer. Importantly, when provided with the input library, the SLISA procedure is as straightforward as ELISA coupled with PCR reactions. The whole process can be performed by simple instrument. With the easy access and deceasing cost of deep sequencing, the SLISA can be broadly applicable.

1. A method for characterizing an immune response to an antigen or a set of antigens in an individual, comprising: generating an immunological profile for an immune response in the individual to the antigen or a set of antigens,
wherein the immunological profile comprises: a multiplicity of nucleic acid sequences of immune receptors from the individual, or a multiplicity of nucleic acid sequences from the antigen, or a combination thereof, and
wherein generating the immunological profile comprises: selecting at least one target binding nucleic acid-protein fusion from a library of two or more nucleic acid-protein fusions,
wherein the target is one or more T cell receptors (TCR) of a target T cell, a B cell receptor (BCR) of a target B cell, or antigen binding domain of a target immunoglobulin (FAb), and
wherein the immunological profile comprises: the nucleic acid sequence of the selected nucleic acid-protein fusion, or the nucleic acid sequence of the target, or both.

2. The method of claim 1, wherein the nucleic acid-protein fusion is generated by RNA display of antigen DNA, or a multiplicity of antigen DNA fragments, wherein the RNA display comprises the steps of
(i) performing in vitro transcription on the antigen DNA or multiplicity of fragments to obtain antigen mRNA(s);
(ii) covalently linking the antigen mRNA(s) at the 3′ end to a protein acceptor selected from the group consisting of puromycin, tRNA-puromycin conjugate, phenylalanyl-adenosine, tyrosyl adenosine, alanyl adenosine, phenylalanyl 3′ deoxy 3′ amino adenosine, alanyl 3′ deoxy 3′ amino adenosine, and tyrosyl 3′ deoxy 3′ amino adenosine to obtain a ligated antigen mRNA; and
(iii) performing in vitro translation and reverse transcription on the ligated antigen mRNA(s) to obtain a nucleic acid-protein fusion(s),
wherein the protein of the nucleic acid-protein fusion is encoded by the nucleic acid of the nucleic acid-protein fusion;
optionally wherein the DNA fragments comprise a promoter sequence, Kozak sequence and a sequence encoding a first peptide tag at the 5′ end, and a sequence encoding a second peptide tag at the 3′ end; and/or wherein the nucleic acid-protein fusion(s) are purified by affinity using the first and/or second peptide tags.

3. The method of claim 1, wherein the immunological profile comprises: peptide epitopes of the antigen that are bound by immunoglobulin from the individual;
wherein the epitopes are between 5 and 100 amino acids in length, between 9 and 60 amino acids in length, or between 30 and 50 amino acids in length;
optionally wherein the epitopes are identified by a method comprising:
(i) immobilizing the immunoglobulin to obtain an immobile phase;
(ii) contacting the immobile phase with the nucleic acid-protein fusions under conditions that allow for binding of the nucleic acid-protein fusions to the immunoglobulin within the immobile phase to obtain target binding nucleic acid-protein fusions; and
(iii) characterizing the target binding nucleic acid-protein fusions;
optionally wherein the target-binding nucleic acid-protein fusions are isolated by binding to immobilized immunoglobulins selected from the group consisting of IgG1, IgG2, IgG3, IgG4, IgM, IgE, and IgA, or combinations thereof, and wherein the immunological profile comprises: information identifying the class of immunoglobulin that bound to each target-binding nucleic acid-protein fusion;
optionally wherein characterizing the target binding nucleic acid-protein fusions comprises: isolating target binding nucleic acid-protein fusions and sequencing the nucleic acid of the nucleic acid-protein fusions; wherein isolating the target binding nucleic acid-protein fusions comprises: eluting the target binding nucleic acid-protein fusion from the immunoglobulin, and wherein the sequencing comprises: PCR amplification and/or bar-coding of the nucleic acid of the target binding nucleic acid-protein fusions and preparation of a sequencing library.

4. The method of claim 3, further comprising: correlating the peptide epitopes of the antigen or the set of antigens within the immunological profile with one or more disease states or indications.

5. The method of claim 1, further comprising: the step of characterizing the target B cell(s);
optionally wherein the target B cell(s) are isolated from a source selected from the group consisting of fresh or properly frozen blood, purified lymphocytes, tissues using selection kits, and organs staining by antibodies recognizing B cell marker;
optionally wherein characterizing the target B cell(s) comprises:
(i) labeling the nucleic acid-protein fusions with a detectable label;
(ii) contacting the labeled nucleic acid-protein fusions with a multiplicity of B cell(s) from the individual,
wherein the contacting is under conditions that allow for binding of the labeled nucleic acid-protein fusions to the target B cell(s); and
(iii) detecting target B cell(s) bound to the labeled nucleic acid-protein fusions;
(iv) isolating the target B cell(s); and
(v) obtaining the nucleic acid sequence of the BCR, and optionally one or more other genes of the target B cell(s);
optionally wherein labeling the nucleic acid-protein fusions with a detectable label comprises: performing reverse transcription of the peptide/protein-nucleic acid-fusion complex using biotin-modified primers, wherein the primers anneal to both the oligo dA or oligo A and constant regions of the mRNA, and wherein the label comprises: fluorophore-conjugated streptavidin that is bound to biotin on the cDNA;
wherein the target B cell(s) are isolated by flow-cytometry, magnetic beads, or other immobilized surfaces,
wherein characterizing the target B cell(s) further comprises preparing one or more databases of a multiplicity of nucleic acid sequences from one or more target B cells, optionally wherein the multiplicity of nucleic acid sequences comprises: the target B cell transcriptome, including but not limited to the BCR sequences.

6. (canceled)

7. The method of claim 1, wherein the molecular profile comprises:
(i) the nucleic acid sequences of a multiplicity of target binding nucleic acid-protein fusions; and
(ii) target B cell data,
wherein the target B cell data comprises: nucleic acid sequences of a multiplicity of target B cell(s) that are bound by the target binding nucleic acid-protein fusions,
wherein the method further comprises associating target B cell data within the immunological profile with an immune response to one or more vaccines or infections associated with the antigen.

8. (canceled)

9. The method of claim 1, further comprising: characterizing the target T cell(s);
wherein characterizing the target T cell(s) comprises:
(i) loading the nucleic acid-protein fusions into major histocompatibility complex (MHC) molecules, wherein the MHC is labeled with a detectable label, to form a multiplicity of labeled MHC/nucleic acid-protein fusions;
(ii) contacting the labeled MHC/nucleic acid-protein fusions with a multiplicity of T cell(s) from the individual, wherein the contacting is under conditions that allow for binding of the labeled MHC/nucleic acid-protein fusions to the target T cell(s); and
(iii) detecting the target T cell(s) bound to the labeled nucleic acid-protein fusions;
(iv) isolating the target T cell(s); and
(v) obtaining the nucleic acid sequence of the TCR, and optionally one or more other genes of the target T cell(s);
optionally wherein characterizing the target T cell(s) further comprises: preparing one or more databases of a multiplicity of nucleic acid sequences from one or more target T cell, optionally wherein the multiplicity of nucleic acid sequences comprises: the target T cell transcriptome.

10. The method of claim 1, wherein the molecular profile comprises:
(i) the nucleic acid sequences of a multiplicity of target binding nucleic acid-protein fusions; and
(ii) target T cell data,
wherein the nucleic acid sequences of a multiplicity of target T cell(s) that are bound by the target binding nucleic acid-protein fusions,
wherein the method further comprises associating target T cell data within the immunological profile with an immune response to one or more vaccines or infections associated with the antigen.

11. (canceled)

12. The method of claim 1, wherein the immunological profile comprises: at least 50% of the BCR repertoire of the individual, or at least 50% of the TCR repertoire of the individual, or at least 50% of the BCR and TCR repertoire of the individual specific for the antigen,
or wherein the immunological profile comprises:
(i) the nucleic acid sequence of target-binding nucleic acid-protein fusions;
(ii) target B cell data; and
(iii) target T cell data.

13. (canceled)

14. The method of claim 1, further comprising: performing one or more computations on the immunological profile to identify one or more criteria within a multiplicity of nucleic acid sequences of immune receptors from the individual, or a multiplicity of nucleic acid sequences from the antigen;
optionally wherein one criterion is identifying an auto-antigen or a set of antigens within the multiplicity of nucleic acid sequences from the antigen, optionally wherein the method further comprises: identifying or assisting selection of anti-autoimmune therapy based on the identification of an auto-antigen;
optionally wherein one criterion is identifying a tumor antigen within the multiplicity of nucleic acid sequences from the antigen, and optionally wherein the method further comprises: identifying or assisting selection of anti-cancer therapy based on the identification of a tumor antigen;
optionally wherein one criterion is identifying a transplantation-associated immune response in the individual, and optionally wherein the method further comprises: identifying or assisting selection of therapy based on the identification of auto-antigens associated with transplant rejection;
optionally wherein one criterion is identifying or diagnosing a disease in the individual, wherein the identifying is based on the identification of immunoglobulins, BCRs and/or TCRs associated with the immune response.

15. The method of claim 1, wherein the target-binding nucleic acid-protein fusions and/or target B cells and/or target T cells comprise one or more synthetic nucleic acid sequences comprising: bar-code information relating to the sample;
optionally wherein the synthetic nucleic acid sequences comprising: bar-code information for target B cell(s) or target T cell(s) are associated with a bead or other matrix with which the target B cell(s) or target T cell(s) is associated.

16. The method of claim 1, wherein the antigen comprises: the SARS-COV-2 virus;
optionally wherein the immunological profile comprises: the nucleic acid sequences of one or more target-binding epitope(s) of the SARS-COV-2 virus, or the nucleic acid sequences of one or more BCR that selectively binds an epitope of the SARS-COV-2 virus, or the nucleic acid sequences of one or more TCR that selectively binds an epitope of the SARS-COV-2 virus, or combinations thereof.

17. A method for identifying antibody epitopes by mRNA display, comprising:
preparation of an mRNA-display epitope library from an antigen; and immuno-capture of the mRNA-display epitope library.

18. The method of claim 17,
(A) wherein preparation of the mRNA-display epitope library comprises:
(i) preparation of a double-stranded DNA library from an antigen,
wherein the double-stranded DNA library comprises: a multiplicity of fragments comprising: a promoter sequence, a nucleic acid motif that functions as a protein translation initiation site, a sequence encoding a first peptide tag at the 5′ end; and a sequence encoding a second peptide tag at the 3′ end;
wherein the promoter sequence is the T7 promoter sequence, the protein translation initiation site is a Kozak sequence, the first peptide tag is a DYKDDDDK tag, and the second peptide tag is a Strep-tagII;
(ii) preparation of a peptide/protein-mRNA fusion complex from the double-stranded DNA library; and
(iii) cDNA synthesis on the peptide/protein-mRNA fusion complex to generate a peptide/protein-mRNA-cDNA fusion complex;
optionally wherein preparation of a peptide/protein-mRNA fusion complex comprises:
(a) in vitro Transcription of the double-stranded DNA to produce RNA,
(b) ligation of the RNA with a poly-dA DNA to produce ligated RNA/DNA,
(c) purification of ligated RNA,
(d) in vitro translation of the ligated RNA/DNA to produce a peptide/protein-mRNA fusion complex, and
(e) purification of the peptide/protein-mRNA fusion complex;
optionally wherein cDNA synthesis to generate peptide/protein-mRNA-cDNA fusion comprises: reverse transcription of the peptide/protein-mRNA fusion to produce a peptide/protein-mRNA-cDNA fusion; and/or
(B) wherein immuno-capture of mRNA-display epitope library comprises:
(i) Capture of antibody from the subject onto a solid matrix;
(ii) Immuno-capture of peptide/protein-mRNA-cDNA fusion;
(iii) Elution of peptide/protein-mRNA-cDNA fusion;
(iv) PCR amplification and barcoding of the peptide/protein-mRNA-cDNA fusion, wherein the barcodes are specific to each sample; and
(vi) Preparation of a sequencing library.

19. (canceled)

20. A method for identifying epitope-specific B cell receptor sequences for an antigen, comprising:
preparation of a labeled mRNA-display epitope library from the antigen;
preparation of B cells labeled with mRNA-display epitope library;
preparation of bar-coded beads;
encapsulation of single B cells and a single-beads into droplet; and
sequencing the library of encapsulated B cells.

21. The method of claim 20,
(A) wherein preparation of the labeled mRNA-display epitope library comprises:
(i) preparation of a double-stranded DNA library from an antigen,
wherein the double-stranded DNA library comprises: a multiplicity of fragments comprising: a promoter sequence, a nucleic acid motif that functions as a protein translation initiation site, a sequence encoding a first peptide tag at the 5′ end; and a sequence encoding a second peptide tag at the 3′ end; optionally wherein the promoter sequence is the T7 promoter sequence, the protein translation initiation site is a Kozak sequence, the first peptide tag is a DYKDDDDK tag, and the second peptide tag is a Strep-tagII;
(ii) preparation of a peptide/protein-mRNA fusion complex from the double-stranded DNA library; and
(iii) cDNA synthesis on the peptide/protein-mRNA fusion complex with fluorescent labeling to generate a labeled peptide/protein-mRNA-cDNA fusion complex;
optionally wherein preparation of a peptide/protein-mRNA fusion complex comprises:
(a) in vitro transcription of the double-stranded DNA to produce RNA,
(b) ligation of the RNA with a poly-dA DNA to produce ligated RNA/DNA,
(c) purification of ligated RNA
(d) in vitro translation of the ligated RNA/DNA to produce a peptide/protein-mRNA fusion complex, and
(e) purification of the peptide/protein-mRNA fusion complex;
optionally wherein cDNA synthesis on the peptide/protein-mRNA fusion complex with fluorescent labeling to generate a labeled peptide/protein-mRNA-cDNA fusion complex comprises:
(a) reverse transcription of the peptide/protein-mRNA fusion using biotin-modified primer to produce a peptide/protein-mRNA-cDNA fusion, wherein the biotin modified primer anneals to both the poly-dA and constant region of the mRNA;
(b) removal of unbound oligos; and
(c) addition of fluorophore-conjugated streptavidin to bind the biotin on the cDNA and to generate a labeled peptide/protein-mRNA-cDNA fusion complex;
(B) wherein preparation of B cells labeled with mRNA-display epitope library comprises:
(i) B Cell preparation; and
(ii) B Cell staining and sorting,
wherein preparation of bar-coded beads comprises:
(iii) Hydrogel bead formation; and
(iv) Split-pool combinatorial barcoding of hydrogel beads;
optionally wherein Hydrogel bead formation comprises: a continuous stream of aqueous phase that is emulsified into a stream of highly mono-disperse droplets that are collected and polymerized into Hydrogel beads;
optionally wherein Hydrogel bead formation is carried out using a microfluidics device;
optionally wherein Split-pool combinatorial barcoding of hydrogel beads comprises: stepwise enzymatic extension and hybridization reactions to add one or more barcoded primers to the beads; more optionally wherein the Split-pool combinatorial barcoding of hydrogel beads is repeated four times to add four barcoded primers to the beads,
(C) wherein Encapsulation of single-cell and single-bead into droplet comprises:
(i) Encapsulation and cDNA synthesis;
(ii) Demulsification and DNA purification;
(iii) cDNA amplification;
(iv) Epitope and B Cell Receptor sequence amplification;
(xii) Fragmentation
(xiii) End-repair/dA-tailing and adaptor ligation; and
(xiv) PCR amplification of the sequencing library, and/or
(D) wherein sequencing the library of encapsulated B cells comprises:
Next Generation Sequencing of the sequencing library to provide an immunological profile;
optionally wherein the immunological profile includes sequences of more than 1000 B cell receptor sequences, or sequences of more than 1000 B cell receptor epitopes, or both.

22-25. (canceled)

26. A method for identifying epitope-specific T cell receptor sequences for an antigen, comprising:
preparation of barcoded MHC tetramers in droplets;
T Cell staining and sorting;
Preparation of barcoded beads; and
Encapsulation of single-cell and single-bead into droplet and sequencing library preparation.

27. The method of claim 26,
(A) wherein preparation of barcoded MHC tetramers in droplets comprises:
(i) Preparation of a double-stranded DNA library from an antigen;
(ii) Preparation of fluorophore and oligo labeled streptavidin (FOS), or fluorophore labeled mono-avidin on branched DNA (FMbD)′; and
(iii) Assembly of barcoded MHC-tetramers/oligomers in droplets;
optionally wherein the preparation of a double-stranded DNA library from an antigen comprises: self-circularization and isothermal amplification to form concatemers of multiple DNA variants;
optionally wherein preparation of fluorophore and oligo labeled streptavidin (FOS) comprises: conjugating a DNA oligo to fluorophore-labeled streptavidin; and conjugation to biotinylated MHC;
optionally wherein the Assembly of barcoded MHC-tetramers/oligomers in droplets comprises:
formation of barcoded MHC-tetramers or MHC-oligomers by in-droplet IVTT reaction, optionally the formation of barcoded MHC-tetramers or MHC-oligomers by in-droplet IVTT reaction comprises: forming droplets of DNA concatemer with an average occupancy of 1 concatemer in 5-10 droplets;
DNA-RNA hybridization; and
cDNA synthesis by reverse transcription,
wherein one or more steps is carried out using a microfluidic device;
optionally wherein the formation of barcoded MHC-tetramers or MHC-oligomers occurs within each droplet in a microfluidic device,
(B) wherein the T cell staining and sorting comprises:
(i) T cell preparation; and
(ii) T Cell staining and sorting;
optionally wherein the T Cell preparation comprises: attaching a barcode or other label to a multiplicity of T cells, wherein each T cell is identified by the barcode or label,
wherein preparation of bar-coded beads comprises:
(iii) hydrogel bead formation; and
(iv) split-pool combinatorial barcoding of hydrogel beads;
optionally wherein hydrogel bead formation comprises: a continuous stream of aqueous phase that is emulsified into a stream of highly mono-disperse droplets that are collected and polymerized into hydrogel beads;
optionally wherein hydrogel bead formation is carried out using a microfluidics device;
optionally wherein split-pool combinatorial barcoding of hydrogel beads comprises: stepwise enzymatic extension and hybridization reactions to add one or more barcoded primers to the beads, optionally wherein the split-pool combinatorial barcoding of hydrogel beads is repeated four times to add four barcoded primers to the beads, and/or (C) wherein encapsulation of single-cell and single-bead into droplet comprises:
(i) Encapsulation and cDNA synthesis;
(ii) Demulsification and DNA purification; and
(iii) cDNA amplification;
(iv) Epitope and T Cell Receptor sequence amplification;
(v) Fragmentation;
(vi) End-repair/dA-tailing and adaptor ligation; and
(vii) PCR amplification of the sequencing library;
optionally wherein sequencing the library of encapsulated T cells comprises: Next Generation Sequencing of the sequencing library to provide an immunological profile, optionally wherein the immunological profile includes sequences of more than 1000 T cell receptor sequences, or sequences of more than 1000 T cell receptor epitopes, or both.

28-31. (canceled)

32. A method of making a vaccine against an antigen for a subject, comprising:
(a) identifying epitope-specific T cell receptor sequences for a multiplicity of epitopes within the antigen,
wherein the epitope-specific T cell receptor sequences for each of the multiplicity of epitopes are determined according to the method of claim 26;
(b) determining which one or more of the multiplicity of epitopes for the antigen have the highest number of epitope-specific T cell receptor sequences; and
(c) preparing the vaccine for the subject including one or more of the epitopes determined in (b).

33. A method of making a vaccine against an antigen for a subject, comprising:
(a) identifying a multiplicity of antibody epitopes within the antigen by mRNA display,
wherein each of the multiplicity of the antibody epitopes are determined according to the method of claim 17; and
(b) preparing the vaccine for the subject including one or more of the epitopes determined in (a).

34. A method of making a vaccine against an antigen for a subject, comprising:
(a) identifying epitope-specific B cell receptor sequences for a multiplicity of epitopes within the antigen,
wherein the epitope-specific B cell receptor sequences for each of the multiplicity of epitopes are determined according to the method of claim 20;
(b) determining which one or more of the multiplicity of epitopes for the antigen have the highest number of epitope-specific B cell receptor sequences; and
(c) preparing the vaccine for the subject including one or more of the epitopes determined in (b).

35. A vaccine prepared according to the method of claim 32.
